(19) United States
(12) Patent Application Publication
(10) Pub. No.: US 2023/0301984 A1
(43) Pub. Date: Sep. 28, 2023

(54) CYCLIC COMPOUNDS FOR USE IN TREATING RETINAL DEGENERATION

(71) Applicant: The USA, as represented by the Secretary, Department of Health and Human Services, Bethesda, MD (US)

(72) Inventors: Anand Swaroop, Gaithersburg, MD (US); Yu Holly Chen, Chevy Chase, MD (US); Manju Swaroop, Gaithersburg, MD (US); Wei Zheng, Potomac, MD (US); Gregory Tawa, Doylestown, PA (US); Anupam Mondal, Rockville, MD (US); Samantha Papal, Garges-les-Gonesse (FR); Wenwei Huang, Rockville, MD (US); Zhiji Luo, Rockville, MD (US)

(73) Assignee: The USA, as represented by the Secretary, Department of Health and Human Services, Bethesda, MD (US)

(21) Appl. No.: 18/014,045

(22) PCT Filed: Jul. 1, 2021
(86) PCT No.: PCT/US2021/040157

§ 371 Date: Dec. 30, 2022

Related U.S. Application Data
(60) Provisional application No. 63/047,858, filed on Jul. 2, 2020.

(51) Int. Cl. A61K 31/475; A61K 31/473; A61P 27/02; A61K 31/498

(52) U.S. Cl. CPC A61K 31/475; A61K 31/473; A61K 31/498; A61P 27/02

(57) ABSTRACT

Method embodiments are disclosed for treating retinal degeneration in a subject in need thereof. In some embodiments, the method comprises administering to the subject a therapeutically effective amount of compound, and/or a pharmaceutically acceptable salt, prodrug, solvate, hydrate, or tautomer thereof, selected from 3-(dibutylamino)-1-(1,3-dichloro-6-(trifluoromethyl)phenanthren-9-yl)propan-1-ol hydrochloride or a compound having a structure according to a formula selected from Formula I, II, or III, as described herein. In some non-limiting examples, the subject has retinitis pigmentosa, LCA, Stargardt’s macular dystrophy, cone-rod dystrophy, choroideremia or age-related macular degeneration.

### CROSS REFERENCE TO RELATED APPLICATION

This application claims the benefit of the earlier priority date of U.S. Provisional Pat. Application No. 63/047,858, filed on Jul. 2, 2020, the entirety of which is incorporated herein by reference.

### ACKNOWLEDGMENT OF GOVERNMENT SUPPORT

This invention was made with government support under ZIAEY000474 and ZIAEY000546 awarded by the National Eye Institute. The government has certain rights in the invention.

### FIELD

The present disclosure concerns the field of retinal degenerative disease, specifically to compounds that are of use for treating retinal degeneration.

### BACKGROUND

The retina is a layer of specialized light sensitive neural tissue located at the inner surface of the eye of vertebrates. Light reaching the retina after passing the cornea, the lens and the vitreous humor is transformed into chemical and electrical events that trigger nerve impulses. The cells that are responsible for transduction, the process for converting light into these biological processes are specialized neurons called photoreceptor cells. Dysfunction and/or degeneration of photoreceptors, the light-sensitive neurons in the retina, are prominent features in diseases of the retina contributing significantly to irreversible blindness worldwide.

Many ophthalmic diseases, such as (age-related) macular degeneration, macular dystrophies such as Stargardt’s and Stargardt’s-like disease, Best disease (vitelliform macular dystrophy), adult vitelliform dystrophy, cone-rod dystrophies, Leber congenital amaurosis and retinitis pigmentosa, are associated with dysfunction, degeneration or deterioration of the retina. It has been demonstrated in some animal models that photoreceptor rescue and preservation of visual function may be achieved by subretinal transplantation of RPE cells or by gene therapy; however, there is a need for compounds of use for the treatment of retinal degenerative diseases.

### SUMMARY

Disclosed herein are embodiments of a method for treating retinal degeneration in a subject. In some embodiments, the method comprises administering to the subject a therapeutically effective amount of 3-(dibutylamino)-1-(1,3-dichloro-6-(trifluoromethyl)phenanthren-9-yl)propan-1-ol hydrochloride or a compound having a structure according to a formula selected from Formula I, II, or III

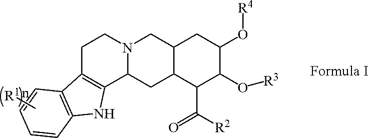

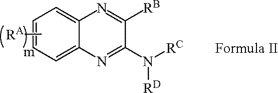

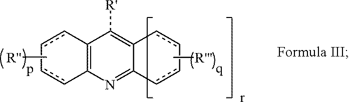

thereby treating the retinal degeneration in the subject; wherein,
with reference to Formula I,
   R¹ is heteroaliphatic;
   R² is OR⁵, or NR⁶R⁷, wherein each of R⁵, R⁶, and R⁷ independently is selected from hydrogen, aliphatic, or aromatic, or an organic functional group;
   each of R³ and R⁴ independently is selected from aliphatic, aromatic, acyl, or sulfonyl; and
   n can be an integer selected from 0 to 4;
with reference to Formula II,
   R^{A}, is selected from halogen, heteroaliphatic, haloaliphatic, or an organic functional group;
   R^{B} is aromatic; and
   each of R^{C} and R^{D} independently is selected from hydrogen, aliphatic, or heteroaliphatic; and
   m is an integer selected from 0 to 4; and
with reference to Formula III
   R′ is selected from aliphatic, aromatic, halogen, heteroaliphatic, haloaliphatic, or an organic functional group;
   each R″ independently is selected from halogen, heteroaliphatic, or amino;
   each R‴ independently is selected from halogen, heteroaliphatic, or amino;
   p is an integer selected from 0 to 4;
   q is an integer selected from 0 to 4; and
   r is an integer selected from 0 or 1.

The foregoing and other objects and features of the present disclosure will become more apparent from the following detailed description, which proceeds with reference to the accompanying figures.

### BRIEF DESCRIPTION OF THE DRAWINGS

FIGS. 1A and 1B are schematic diagrams showing the typical autophagy pathway associated with retinal ciliopathies (FIG. 1A) and a proposed model of the mechanism of action of compound embodiments disclosed (FIG. 1B).

FIGS. 2A-2G show results from analyzing disease-associated phenotypes in CEP290-LCA subject induced pluripotent stem cell-derived retinal organoids; FIG. 2A shows immunostaining of RHO (Rhodopsin, magenta), OPN1SW (S-opsin, red), OPN1M/LW (L/M-opsin, green) and FIG. 2B shows immunostaining of RHO (Rhodopsin, green), and ARL13B (ADP-ribosylation factor-like protein 13B, red), wherein the nuclei are stained by DAPI and images are representative of 2 cell lines for each subject, each of which had at least 6 batches of experiments, with at least 6 retinal organoids in each experiment; FIG. 2C shows principle component analysis of gene profiles of control and subject iPSC-derived retinal organoids at D67, D90, D120 and D150; FIG. 2D shows a summary of differentially expressed (DE) genes between control and subject organoids across development; FIG. 2E provides a Venn diagram showing DE genes in development and age-matched pairwise comparison of control and subject samples; FIG. 2F shows KEGG and Reactome pathway analysis of DE genes unique to CEP290 mutations in subject samples; and FIG. 2G shows that expression of phototransduction genes was mostly down-regulated in subject organoids.

FIG. 3 shows a schematic diagram of a compound discovery pipeline, wherein approximately 6000 compounds with various concentrations were applied to dissociated cells including photoreceptors from rd16 mouse (a model of CEP290-LCA) retinal organoids and putative compound embodiments were selected by various criteria and the hits were validated by using mouse and then human retinal organoids (transcriptome analyses are performed to elucidate their action mechanisms and their effects are tested in degenerative mouse retina in vivo).

FIGS. 4A-4D show results associated with compound embodiments that improved photoreceptor development in rd16 induced pluripotent stem cell (iPSC)-derived retinal organoids; FIG. 4A shows a schematic diagram of small molecule treatment in rd16 retinal organoids; FIG. 4B shows the immunostaining of Rhodopsin (green) and S-opsin (red), wherein nuclei are stained by DAPI and images are representative of at least 3 batches of experiments, each of which had at least 3 retinal organoids; and FIGS. 4C and 4D show the quantification of fluorescence intensity of Rhodopsin (FIG. 4C) and S-opsin staining (FIG. 4D) of untreated and treated rd16 retinal organoids from at least 3 batches of experiments, each of which had at least 3 retinal organoids.

FIGS. 5A-5C show results confirming improved photoreceptor and cilia biogenesis by compound embodiments in subject induced pluripotent stem cell (iPSC)-derived retinal organoids; FIG. 5A shows a schematic diagram of small molecule treatment in subject retinal organoids; FIG. 5B shows immunostaining of rod cell marker Rhodopsin (green) and ciliary axoneme marker ARL13B (red); and FIG. 5C shows S-cones and L/M-cones were shown by immunostaining of OPN1L/MW (green) and OPN1SW (red), wherein nuclei are stained by DAPI and images are representative of two cell lines for each subject, each of which had at least 3 batches of experiments with at least 3 retinal organoids in each batch.

FIGS. 6A and 6B show results confirming that an injected compound embodiment (reserpine) maintained outer nuclear layer of rd16 mice when administered in vivo; FIG. 6A shows a schematic diagram of intravitreal injection in rd16 mice; and FIG. 6B shows immunostaining of rod cell marker Rhodopsin (magenta), PDEβ (red) and GFP (green), wherein GFP (green) signifies rod photoreceptors in the outer nuclear layer and Rhodopsin (magenta) and PDEβ (red) are ciliary proteins located in the outer segments of photoreceptors (nuclei are stained by DAPI and images are representative of 2 out of 3 injected animals).

FIGS. 7A-7E show results obtained from evaluating the drug effect on subject retinal organoids; FIG. 7A shows the timeline for the drug treatment on CEP290-LCA (IVS26+1655A>G p.C998X; c.5668G>T p.G1890X); FIG. 7B shows Western blot analyses of rhodopsin level in subject organoids; FIG. 7C shows a graph of relative fold change as a function of dose that quantifies the rhodopsin level in subject organoids; FIGS. 7D and 7E show images obtained from immunostaining of rod (rhodopsin, green), S-cone (S-opsin, red), L/M-cone (L/M-opsin, magenta) photoreceptors (upper panel) and ciliary axoneme (ARL13B, red) (lower panel) for subjects 1 and 2, respectively (nuclei are stained by DAPI and images are representative of at least 3 batches of experiments, each of which had at least 3 retinal organoids; arrowheads indicate relevant staining).

FIGS. 8A-8G show results obtained from analyzing misregulation of autophagy in subject organoids; FIG. 8A shows a simplified schematic diagram of autophagy; FIG. 8B shows the timeline for the analyses; FIG. 8C shows Western blot analyses of certain autophagy components (p-ULK1 Ser757, ULK1, p62, and LC3-II) in subject organoids; and FIGS. 8D-8G are bar graphs of relative protein amount as a function of time showing quantification of these autophagy components in subject organoids.

FIGS. 9A and 9B show results associated with applying autophagy inhibitors on subject organoids; FIG. 9A shows a schematic diagram illustrating the effects of applying FDA-approved autophagy inhibitor drugs on subject organoids; FIG. 9B shows results from immunostaining of rod (rhodopsin, green), S-cone (S-opsin, red) and L/M-cone (L/M-opsin, magenta) photoreceptors (nuclei stained by DAPI and images are representative of 2 batches of experiments, each of which had at least 6 retinal organoids).

FIGS. 10A-10G show results obtained from analyzing the ability of p62 to act as a mediator for the drug effect of reserpine; FIG. 10A shows Western blot analyses of p62 and LC3-II; FIG. 10B includes bar graphs of relative protein amount as a function of treatment status showing quantification of p62 and LC3-II; FIG. 10C shows results from immunostaining of p62 and acetylated tubulin (DM1T) in treated subject organoids (nuclei are stained by DAPI and images are representative of at least 2 batches of experiments, each of which had at least 3 retinal organoids); FIG. 10D shows Western blot analyses of p62 interaction partner/cilium disassembly key driver HDAC6 and other ciliary regulatory proteins including IFT88 (intraflagellar transport), BBS6 and CEP164 (distal appendage component for initiation of ciliogenesis) in treated organoidsdand quantification; FIG. 10E includes bar graphs of relative protein amount as a function of treatment status showing quantification of these proteins; FIG. 10F shows TEM images of organoids showing reduced defects in docking of preciliary vesicles and ciliary membrane formation due to treatment with reserpine (top panel) and that exhibit longer ciliary axoneme in treated photoreceptors (lower panel); and FIG. 10G shows TEM images of the organoids and a well-organized disc-like structure, which is rare in organoid culture, indicating a favorable effect of reserpine on developing an outer segment (primary cilium of photoreceptors).

FIGS. 11A and 11B show results that indicating an improved photoreceptor morphology after short-term treatment of CEP290-LCA subject induced pluripotent stem cell-derived retinal organoids; FIG. 11A is a schematic diagram showing the small molecule treatment paradigm for CEP290-LCA retinal organoids; and FIG. 11B shows immunostaining of rod cells (green), S-cones (red) and L/M-cones (magenta) (nuclei are stained by DAPI and images are representative of 2 batches of experiments, each of which had at least 3 retinal organoids).

### DETAILED DESCRIPTION

### I. Explanation of Terms

The following explanations of terms are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting, unless otherwise indicated. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from discussed prior art, the embodiment numbers are not approximates unless the word “about” is recited. Furthermore, not all alternatives recited herein are equivalents.

Compound embodiments disclosed herein may contain one or more asymmetric elements such as stereogenic centers, stereogenic axes and the like, e.g., asymmetric carbon atoms, so that the chemical conjugates can exist in different stereoisomeric forms. These compound embodiments can be, for example, racemates or optically active forms. For compound embodiments with two or more asymmetric elements, these compound embodiments can additionally be mixtures of diastereomers. For compound embodiments having asymmetric centers, all optical isomers in pure form and mixtures thereof are encompassed by corresponding generic formulas unless context clearly indicates otherwise or an express statement excluding an isomer is provided. In these situations, the single enantiomers, i.e., optically active forms can be obtained by method known to a person of ordinary skill in the art, such as asymmetric synthesis, synthesis from optically pure precursors, or by resolution of the racemates. Resolution of the racemates can also be accomplished, for example, by conventional methods, such as crystallization in the presence of a resolving agent, or chromatography, using, for example a chiral HPLC column. All isomeric forms are contemplated herein regardless of the methods used to obtain them.

All forms (for example solvates, optical isomers, enantiomeric forms, polymorphs, free compound and salts) of an active agent may be employed either alone or in combination. Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes (+/-) D and L or R and S are used to denote the absolute configuration of the molecule about its chiral center(s). The prefixes d and 1 or (+) and (-) are employed to designate the sign of rotation of plane-polarized light by the compound, with (-) or 1 meaning that the compound is levorotatory. A compound prefixed with (+) or d is dextrorotatory.

To facilitate review of the various embodiments of the disclosure, the following explanations of specific terms are provided. Certain functional group terms include a symbol “-” which is used to show how the defined functional group attaches to, or within, the compound to which it is bound.

Also, a dashed bond (i.e., “---”) as used in certain formulas described herein indicates an “optional” bond to a substituent or atom of the formula other than hydrogen in the sense that the bond (and in some embodiments, the substituent) may or may not be present. In any formulas comprising a dashed bond, if the optional bond and/or any corresponding substituent is not present, then the valency requirements of any atom(s) bound thereto is completed by a bond to a hydrogen atom. Solely by way of example, in the following formula, the dashed bond between the carbon atom of the pyridine ring and the R′ group may be present, or this bond and R′ substituent may be absent and instead a bond to a hydrogen atom is present. Also, the dashed bonds in the fused rings of the formula indicate that double bonds may be present, or not present, in which case a single bond is present and the corresponding carbon atoms are bound to hydrogen atoms, in addition to any other substituents already bound thereto. Furthermore, with respect to this particular formula, if r is 0 (as provided herein), then the valency of each pyridine carbon atom is instead satisfied by a bond to hydrogen as opposed to a fused ring.

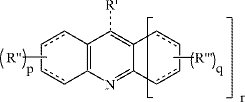

The symbol

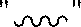

is used to indicate a bond disconnection in abbreviated structures/formulas provided herein. A person of ordinary skill in the art would recognize that the definitions provided below and the compounds and formulas included herein are not intended to include impermissible substitution patterns (e.g., methyl substituted with 5 different groups, and the like). Such impermissible substitution patterns are easily recognized by a person of ordinary skill in the art. In formulas and compounds disclosed herein, a hydrogen atom is present and completes any formal valency requirements (but may not necessarily be illustrated) wherever a functional group or other atom is not illustrated. For example, a phenyl ring that is drawn as

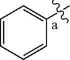

comprises a hydrogen atom attached to each carbon atom of the phenyl ring other than the “a” carbon, even though such hydrogen atoms are not illustrated. Any functional group disclosed herein and/or defined above can be substituted or unsubstituted, unless otherwise indicated herein. Any compound embodiment described herein can be deuterated or not deuterated, unless otherwise indicated herein. Suitable positions at which a compound can be deuterated are readily recognized by people of ordinary skill in the art.

A person of ordinary skill in the art will appreciate that compounds may exhibit the phenomena of tautomerism, conformational isomerism, geometric isomerism, and/or optical isomerism. For example, certain disclosed compounds can include one or more chiral centers and/or double bonds and as a consequence can exist as stereoisomers, such as double-bond isomers (i.e., geometric isomers), enantiomers, diastereomers, and mixtures thereof, such as racemic mixtures. As another example, certain disclosed compounds can exist in several tautomeric forms, including the enol form, the keto form, and mixtures thereof. As the various compound names, formulae and compound drawings within the specification and claims can represent only one of the possible tautomeric, conformational isomeric, optical isomeric, or geometric isomeric forms, a person of ordinary skill in the art will appreciate that the disclosed compounds encompass any tautomeric, conformational isomeric, optical isomeric, and/or geometric isomeric forms of the compounds described herein, as well as mixtures of these various different isomeric forms. Mixtures of different isomeric forms, including mixtures of enantiomers and/or stereoisomers, can be separated to provide each separate enantiomers and/or stereoisomer using techniques known to those of ordinary skill in the art, particularly with the benefit of the present disclosure. In cases of limited rotation, e.g. around the amide bond or between two directly attached rings such as pyridinyl rings, biphenyl groups, and the like, atropisomers are also possible and are also specifically included in the compounds disclosed herein.

In any embodiments, any or all hydrogens present in the compound, or in a particular group or moiety within the compound, may be replaced by a deuterium or a tritium. Thus, a recitation of alkyl includes deuterated alkyl, where from one to the maximum number of hydrogens present may be replaced by deuterium. For example, methyl refers to both CH₃ or CH₃ wherein from 1 to 3 hydrogens are replaced by deuterium, such as in CDₓH₃₋ₓ.

As used herein, the term “substituted” refers to all subsequent modifiers in a term, for example in the term “substituted aliphatic-aromatic,” substitution may occur on the “aliphatic” portion, the “aromatic” portion or both portions of the aliphatic-aromatic group.

“Substituted,” when used to modify a specified group or moiety, means that at least one, and perhaps two or more, hydrogen atoms of the specified group or moiety is independently replaced with the same or different substituent groups. In a particular embodiment, a group, moiety, or substituent may be substituted or unsubstituted, unless expressly defined as either “unsubstituted” or “substituted.” Accordingly, any of the functional groups specified herein may be unsubstituted or substituted unless the context indicates otherwise or a particular structural formula precludes substitution. In particular embodiments, a substituent may or may not be expressly defined as substituted but is still contemplated to be optionally substituted. For example, an “aliphatic” or a “cyclic” moiety may be unsubstituted or substituted, but an “unsubstituted aliphatic” or an “unsubstituted cyclic” is not substituted. In one embodiment, a group that is substituted has at least one substituent up to the number of substituents possible for a particular moiety, such as 1 substituent, 2 substituents, 3 substituents, or 4 substituents.

Any group or moiety defined herein can be connected to any other portion of a disclosed structure, such as a parent or core structure, as would be understood by a person of ordinary skill in the art, such as by considering valence rules, comparison to exemplary species, and/or considering functionality, unless the connectivity of the group or moiety to the other portion of the structure is expressly stated, or is implied by context.

Acyl: -C(O)R^{a}, wherein R^{a} is selected from aliphatic, heteroaliphatic, haloaliphatic, haloheteroaliphatic, aromatic, or an organic functional group.

Acyl Halide: —C(O)X, wherein X is a halogen, such as Br, F, I, or Cl.

Age-related macular degeneration (AMD): A disease that is a major cause of blindness in the United States and other industrialized nations. (Evans J, Wormald R., British Journal Ophthalmology 80:9-14, 1996; Klein R, Klein B E K, Linton K L P, Ophthalmology 99:933-943, 1992; Vingerling J R, Ophthalmology 102:205-210, 1995). Early AMD is characterized clinically by drusen, which are extracellular deposits of proteins, lipids, and cellular debris, (Hageman G S, Mullins R F, Mol Vis 5:28, 1999), that are located beneath the retinal pigment epithelium (RPE). The RPE provides nutritional, metabolic, and phagocytic functions for the overlying photoreceptors. Significant vision loss results from dysfunction or death of photoreceptors in the macula in association with late stages of AMD (geographic atrophy of the retinal pigment epithelial cells and subretinal neovascularization).

Aldehyde: —C(O)H.

Aliphatic: A hydrocarbon group having at least one carbon atom to 50 carbon atoms (C₁₋₅₀), such as one to 25 carbon atoms (C₁₋₂₅), or one to ten carbon atoms (C₁₋₁₀), and which includes alkanes (or alkyl), alkenes (or alkenyl), alkynes (or alkynyl), including cyclic versions thereof, and further including straight-and branched-chain arrangements, and all stereo and position isomers as well.

Alkenyl: An unsaturated monovalent hydrocarbon having at least two carbon atom to 50 carbon atoms (C₂₋₅₀), such as two to 25 carbon atoms (C₂₋₂₅), or two to ten carbon atoms (C₂₋₁₀), and at least one carbon-carbon double bond, wherein the unsaturated monovalent hydrocarbon can be derived from removing one hydrogen atom from one carbon atom of a parent alkene. An alkenyl group can be branched, straight-chain, cyclic (e.g., cycloalkenyl), cis, or trans (e.g., E or Z).

Alkoxy: -O-aliphatic, such as -O-alkyl, -O-alkenyl, -O-alkynyl; with exemplary embodiments including, but not limited to, methoxy, ethoxy, n-propoxy, isopropoxy, n-butoxy, t-butoxy, sec-butoxy, n-pentoxy (wherein any of the aliphatic components of such groups can comprise no double or triple bonds, or can comprise one or more double and/or triple bonds).

Alkyl: A saturated monovalent hydrocarbon having at least one carbon atom to 50 carbon atoms (C₁₋₅₀), such as one to 25 carbon atoms (C₁₋₂₅), or one to ten carbon atoms (C₁₋₁₀),, wherein the saturated monovalent hydrocarbon can be derived from removing one hydrogen atom from one carbon atom of a parent compound (e.g., alkane). An alkyl group can be branched, straight-chain, or cyclic (e.g., cycloalkyl).

Alkynyl: An unsaturated monovalent hydrocarbon having at least two carbon atom to 50 carbon atoms (C₂₋₅₀), such as two to 25 carbon atoms (C₂₋₂₅), or two to ten carbon atoms (C₂₋₁₀), and at least one carbon-carbon triple bond, wherein the unsaturated monovalent hydrocarbon can be derived from removing one hydrogen atom from one carbon atom of a parent alkyne. An alkynyl group can be branched, straight-chain, or cyclic (e.g., cycloalkynyl).

Amide: -C(O)NR^{a}R^{b} or -NR^{a}C(O)R^{b} wherein each of R^{a} and R^{b} independently is selected from hydrogen, aliphatic, heteroaliphatic, haloaliphatic, haloheteroaliphatic, aromatic, or an organic functional group.

Amino: -NR^{a}R^{b}, wherein each of R^{a} and R^{b} independently is selected from hydrogen, aliphatic, heteroaliphatic, haloaliphatic, haloheteroaliphatic, aromatic, or an organic functional group.

Aromatic: A cyclic, conjugated group or moiety of, unless specified otherwise, from 5 to 15 ring atoms having a single ring (e.g., phenyl) or multiple condensed rings in which at least one ring is aromatic (e.g., naphthyl, indolyl, or pyrazolopyridinyl); that is, at least one ring, and optionally multiple condensed rings, have a continuous, delocalized π-electron system. Typically, the number of out of plane π-electrons corresponds to the Hückel rule (4n + 2). The point of attachment to the parent structure typically is through an aromatic portion of the condensed ring system. For example,

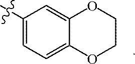

However, in certain examples, context or express disclosure may indicate that the point of attachment is through a non-aromatic portion of the condensed ring system. For example,

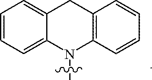

An aromatic group or moiety maycomprise only carbon atoms in the ring, such as in an aryl group or moiety, or it may comprise one or more ring carbon atoms and one or more ring heteroatoms comprising a lone pair of electrons (e.g. S, O, N, P, or Si), such as in a heteroaryl group or moiety. Aromatic groups may be substituted with one or more groups other than hydrogen, such as aliphatic, heteroaliphatic, haloaliphatic, haloheteroaliphatic, aromatic, or an organic functional group.

Aryl: An aromatic carbocyclic group comprising at least five carbon atoms to 15 carbon atoms (C₅-C₁₅), such as five to ten carbon atoms (C₅-C₁₀), having a single ring or multiple condensed rings, which condensed rings can or may not be aromatic provided that the point of attachment to a remaining position of the compounds disclosed herein is through an atom of the aromatic carbocyclic group. Aryl groups may be substituted with one or more groups other than hydrogen, such as aliphatic, heteroaliphatic, haloaliphatic, haloheteroaliphatic, aromatic, or an organic functional group.

Aroxy: -O-aromatic.

Azo: -N=NR^{a} wherein R^{a} is hydrogen, aliphatic, heteroaliphatic, haloaliphatic, haloheteroaliphatic, aromatic, or an organic functional group.

Carbamate: -OC(O)NR^{a}R^{b}, wherein each of R^{a} and R^{b} independently is selected from hydrogen, aliphatic, heteroaliphatic, haloaliphatic, haloheteroaliphatic, aromatic, or an organic functional group.

Carboxyl: —C(O)OH.

Carboxylate: —C(O)O⁻ or salts thereof, wherein the negative charge of the carboxylate group may be balanced with an M⁺ counterion, wherein M⁺ may be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Carrier: An excipient that serves as a component capable of delivering a compound described herein. In some embodiments, a carrier can be a suspension aid, solubilizing aid, or aerosolization aid. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In some examples, the pharmaceutically acceptable carrier may be sterile to be suitable for administration to a subject (for example, by parenteral, intramuscular, or subcutaneous injection). In addition to biologically-neutral carriers, pharmaceutical formulations to be administered can contain minor amounts of non-toxic auxiliary substances, such as wetting or emulsifying agents, preservatives, and pH buffering agents and the like, for example sodium acetate or sorbitan monolaurate.

Chorioderemia: An X-lined recessive form of hereditary retinal degeneration that affect males. The disease causes a gradual loss of vision, starting with childhood night blindness, followed by peripheral vision loss and progressing to loss of central vision later in life. Choroideremia is caused by a loss-of-function mutation in the CHM gene which encodes Rab escort protein 1 (REP1), a protein involved in lipid modification of Rab proteins. The first symptom many individuals with choroideremia notice is a significant loss of night vision. Peripheral vision loss occurs gradually, starting as a ring of vision loss, and continuing on to “tunnel vision” in adulthood. Individuals with choroideremia tend to maintain good visual acuity into their 40s, but eventually lose all sight when they are 50-70 years of age.

Cone-Rod Dystrophy: The first signs and symptoms of cone-rod dystrophy, which often occur in childhood, are usually decreased sharpness of vision (visual acuity) and increased sensitivity to light (photophobia). These features are typically followed by impaired color vision (dyschromatopsia), blind spots (scotomas) in the center of the visual field, and partial side (peripheral) vision loss. Over time, affected individuals develop night blindness and a worsening of their peripheral vision, which can limit independent mobility. The cone dystrophy is characterized by progressive dysfunction of the photopic system, with preservation of scotopic function. Abnormal rod function may be part of the initial presentation, but rod involvement may be less severe, or occur later than the cone dysfunction. There are more than 30 types of cone-rod dystrophy, which are distinguished by their genetic cause and their pattern of inheritance: autosomal recessive, autosomal dominant, and X-linked. Mutations in more than 30 genes are known to cause cone-rod dystrophy. Approximately 20 of these genes are associated with the form of cone-rod dystrophy that is inherited in an autosomal recessive pattern. Mutations in the GUCY2D and CRX genes account for about half of the autosomal dominant form of this disease.

Cyano: —CN.

Disulfide: -SSR^{a}, wherein R^{a} is selected from hydrogen, aliphatic, heteroaliphatic, haloaliphatic, haloheteroaliphatic, aromatic, or an organic functional group.

Dithiocarboxylic: -C(S)SR^{a} wherein R^{a} is selected from hydrogen, aliphatic, heteroaliphatic, haloaliphatic, haloheteroaliphatic, aromatic, or an organic functional group.

Effective Amount: A quantity of a specified pharmaceutical or therapeutic agent sufficient to achieve a desired effect in a subject, or in a cell, being treated with the agent. The effective amount of the agent, such as a nucleic acid molecule, will be dependent on several factors, including, but not limited to the subject or cells being treated, and the manner of administration of the therapeutic composition. An effective amount can be the amount sufficient to treat a subject with a retinopathy.

Ester: -C(O)OR^{a} or -OC(O)R^{a}, wherein R^{a} is selected from aliphatic, heteroaliphatic, haloaliphatic, haloheteroaliphatic, aromatic, or an organic functional group.

Ether: -aliphatic-O-aliphatic, -aliphatic-O-aromatic, -aromatic-O-aliphatic, or -aromatic-O-aromatic.

Halo (or halide or halogen): Fluoro, chloro, bromo, or iodo.

Haloaliphatic: An aliphatic group wherein one or more hydrogen atoms, such as one to 10 hydrogen atoms, independently is replaced with a halogen atom, such as fluoro, bromo, chloro, or iodo.

Haloalkyl: An alkyl group wherein one or more hydrogen atoms, such as one to 10 hydrogen atoms, independently is replaced with a halogen atom, such as fluoro, bromo, chloro, or iodo. In an independent embodiment, haloalkyl can be a CX₃ group, wherein each X independently can be selected from fluoro, bromo, chloro, or iodo.

Heteroaliphatic: An aliphatic group comprising at least one heteroatom to 20 heteroatoms, such as one to 15 heteroatoms, or one to 5 heteroatoms, which can be selected from, but not limited to oxygen, nitrogen, sulfur, silicon, boron, selenium, phosphorous, and oxidized forms thereof within the group. Alkoxy, ether, amino, disulfide, peroxy, and thioether groups are exemplary (but non-limiting) examples of heteroaliphatic. In some embodiments, a fluorophore can also be described herein as a heteroaliphatic group, such as when the heteroaliphatic group is a heterocyclic group.

Heteroaryl: An aryl group comprising at least one heteroatom to six heteroatoms, such as one to four heteroatoms, which can be selected from, but not limited to oxygen, nitrogen, sulfur, silicon, boron, selenium, phosphorous, and oxidized forms thereof within the ring. Such heteroaryl groups can have a single ring or multiple condensed rings, wherein the condensed rings may or may not be aromatic and/or contain a heteroatom, provided that the point of attachment is through an atom of the aromatic heteroaryl group. Heteroaryl groups may be substituted with one or more groups other than hydrogen, such as aliphatic, heteroaliphatic, haloaliphatic, haloheteroaliphatic, aromatic, or an organic functional group. In some embodiments, a fluorophore can also be described herein as a heteroaryl group.

Heteroatom: An atom other than carbon or hydrogen, such as (but not limited to) oxygen, nitrogen, sulfur, silicon, boron, selenium, or phosphorous. In particular disclosed embodiments, such as when valency constraints do not permit, a heteroatom does not include a halogen atom.

Inhibiting: Inhibiting the full development of a disease or condition, for example, in a subject who is at risk for a disease such as a retinopathy, such as, but not limited to, LCA or AMD.

Intraocular administration: Administering agents locally, directly into the eye, for example by delivery into the vitreous or anterior chamber, or sub-retinally. Indirect intraocular delivery (for example by diffusion through the cornea) is not direct administration into the eye.

Intravitreal administration: Administering agents into the vitreous cavity. The vitreous cavity is the space that occupies most of the volume of the core of the eye with the lens and its suspension system (the zonules) as its anterior border and the retina and its coating as the peripheral border. Intravitreal administration can be accomplished by injection, pumping, or by implants.

Leber congenital amaurosis (LCA): A rare inherited eye disease that appears at birth or in the early stages of life (infancy or early childhood) and primarily affects the retina. The presentation can vary because is it associated with multiple genes. However, it is characterized by nystagmus, photophobia, sluggish or absent pupillary response, and severe vision loss or blindness. The common modes of inheritance are autosomal recessive and autosomal dominant.

The pupils, which usually expand and contract in response to the amount of light entering the eye, do not react normally to light. Instead, they expand and contract more slowly than normal, or they may not respond to light at all. Additionally, the clear front covering of the eye (the cornea) may be cone-shaped and abnormally thin, a condition known as keratoconus.

A specific behavior called Franceschetti’s oculo-digital sign is characteristic of Leber congenital amaurosis. This sign consists of poking, pressing, and rubbing the eyes with a knuckle or finger.

Opsin: A member of a group of proteins, made light-sensitive, via the chromophore retinal (or a variant) found in photoreceptor cells of the retina. Opsins are phototransduction proteins. Mammalian opsins are seven transmembrane proteins of the G-protein receptor superfamily. Ciliary (c) opsins, found in vertebrates and cnidarians, attach to ciliary structures such as rods and cones. Rhabdomeric opsins are attached to light-gathering organelles called rhabdomeres. Ciliary opsins (or c-opsins) are expressed in ciliary photoreceptor cells and include the vertebrate visual opsins and encephalopsins. These opsins convert light signals to nerve impulses via cyclic nucleotide gated ion channels, which work by increasing the charge differential across the cell membrane (hyperpolarization). Vertebrates typically have four cone opsins (long wave sensitive (LWS), short wave sensitive (SWS)1, SWS2, and rhodopsin like (Rh)2) inherited from the first vertebrate (and thus predating the first vertebrate), as well as the rod opsin, rhodopsin (Rh1). In humans, RHO is an opsin expressed in rod cells. Human cone cells express:
a) Long-wavelength sensitive (OPN1LW) Opsin: λmax of 560 nm, in the yellow-green region of the electromagnetic spectrum, also called the “red opsin,” “erythrolabe,” “L opsin” or “LWS opsin.”
b) Middle-wavelength sensitive (OPN1MW) Opsin: λmax of 530 nm, in the green region of the electromagnetic spectrum, also called “green opsin,” “chlorolabe,” “M opsin” or “MWS opsin.”
c) Short-wavelength sensitive (OPN1SW) Opsin: λmax of 430 nm, in the blue region of the electromagnetic spectrum, also called the “blue opsin,” “cyanolabe,” “S opsin,” “Opsin-S” or “SWS opsin.”

Organic Functional Group: A functional group that may be provided by any combination of aliphatic, heteroaliphatic, aromatic, haloaliphatic, and/or haloheteroaliphatic groups, or that may be selected from, but not limited to, aldehyde; aroxy; acyl halide; nitro; cyano; azide; carboxyl (or carboxylate); amide; acyl; carbonate; imine; azo; carbamate; hydroxyl; thiol; sulfonyl (or sulfonate); oxime; ester; thiocyanate; thioacyl; thiocarboxylic acid; thioester; dithiocarboxylic acid or ester; phosphonate; phosphate; silyl ether; sulfinyl; thial; or combinations thereof.

Oxime: -CR^{a}=NOH, wherein R^{a} is hydrogen, aliphatic, heteroaliphatic, haloaliphatic, haloheteroaliphatic, aromatic, or an organic functional group.

Peroxy: -O-OR^{a} wherein R^{a} is hydrogen, aliphatic, heteroaliphatic, haloaliphatic, haloheteroaliphatic, aromatic, or an organic functional group.

Pharmaceutically Acceptable Excipient: A substance, other than a compound that is included in a formulation of the compound. As used herein, an excipient may be incorporated within particles of a pharmaceutical composition, or it may be physically mixed with particles of a pharmaceutical composition. An excipient also can be in the form of a solution, suspension, emulsion, or the like. An excipient can be used, for example, to dilute an active agent and/or to modify properties of a pharmaceutical composition. Excipients can include, but are not limited to, antiadherents, binders, coatings, enteric coatings, disintegrants, flavorings, sweeteners, colorants, lubricants, glidants, sorbents, preservatives, adjuvants, carriers or vehicles. Excipients may be starches and modified starches, cellulose and cellulose derivatives, saccharides and their derivatives such as disaccharides, polysaccharides and sugar alcohols, protein, synthetic polymers, crosslinked polymers, antioxidants, amino acids or preservatives. Exemplary excipients include, but are not limited to, magnesium stearate, stearic acid, vegetable stearin, sucrose, lactose, starches, hydroxypropyl cellulose, hydroxypropyl methylcellulose, xylitol, sorbitol, maltitol, gelatin, polyvinylpyrrolidone (PVP), polyethylene glycol (PEG), tocopheryl polyethylene glycol 1000 succinate (also known as vitamin E TPGS, or TPGS), carboxy methyl cellulose, dipalmitoyl phosphatidyl choline (DPPC), vitamin A, vitamin E, vitamin C, retinyl palmitate, selenium, cysteine, methionine, citric acid, sodium citrate, methyl paraben, propyl paraben, sugar, silica, talc, magnesium carbonate, sodium starch glycolate, tartrazine, aspartame, benzalkonium chloride, sesame oil, propyl gallate, sodium metabisulphite or lanolin. In independent embodiments, water is not intended as a pharmaceutically acceptable excipient.

Pharmaceutically Acceptable Salt: Pharmaceutically acceptable salts of a compound described herein that are derived from a variety of organic and inorganic counter ions as will be known to a person of ordinary skill in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like. “Pharmaceutically acceptable acid addition salts” are a subset of “pharmaceutically acceptable salts” that retain the biological effectiveness of the free bases while formed by acid partners. In particular, the disclosed compound embodiments form salts with a variety of pharmaceutically acceptable acids, including, without limitation, inorganic acids such as hydrochloric acid, hydrobromic acid, sulfuric acid, nitric acid, phosphoric acid, and the like, as well as organic acids such as formic acid, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, benzene sulfonic acid, isethionic acid, methanesulfonic acid, ethanesulfonic acid, p-toluenesulfonic acid, salicylic acid, and the like. “Pharmaceutically acceptable base addition salts” are a subset of “pharmaceutically acceptable salts” that are derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, S. M. Berge, et al., “Pharmaceutical Salts,” J. Pharm. Sci., 1977; 66:1-19 which is incorporated herein by reference.)

Phosphate: -O-P(O)(OR^{a})₂, wherein each R^{a} independently is hydrogen, aliphatic, heteroaliphatic, haloaliphatic, haloheteroaliphatic, aromatic, or an organic functional group; or wherein one or more R^{a} groups are not present and the phosphate group therefore has at least one negative charge, which can be balanced by a counterion, M⁺, wherein each M⁺ independently can be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Phosphonate: -P(O)(OR^{a})₂, wherein each R^{a} independently is hydrogen, aliphatic, heteroaliphatic, haloaliphatic, haloheteroaliphatic, aromatic, or an organic functional group; or wherein one or more R^{a} groups are not present and the phosphate group therefore has at least one negative charge, which can be balanced by a counterion, M⁺, wherein each M⁺ independently can be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Photic Retinopathy: Damage to the retina, such as the macula, from prolonged exposure to solar radiation or other bright light, e.g. lasers or arc welders. The term includes solar, laser, and welder’s retinopathy. In some embodiments, photic retinopathy is caused by intense artificial light or sunlight. The light can be ultraviolet light (UV-B, 295-320 nm; UV-A, 320-400 nm) or visible light (400-700 nm). Phototoxic damage can occur in retinal pigment epithelial cells, the choroid, and the rod outer segments. Photic retinopathy results in reduced visual acuity in the long-term, and central or paracentral scotoma. Fundus changes are usually (but not always) bilateral

Prodrug: Compound embodiments disclosed herein that are transformed, most typically in vivo, to yield a biologically active compound, particularly the parent compound, for example, by hydrolysis in the gut or enzymatic conversion. Common examples of prodrug moieties include, but are not limited to, pharmaceutically acceptable ester and amide forms of a compound having an active form bearing a carboxylic acid moiety. Examples of pharmaceutically acceptable esters of the compound embodiments of the present disclosure include, but are not limited to, esters of phosphate groups and carboxylic acids, such as aliphatic esters, particularly alkyl esters (for example C₁₋₆alkyl esters). Other prodrug moieties include phosphate esters, such as -CH₂-O-P(O)(OR^{a})₂ or a salt thereof, wherein R^{a} is hydrogen or aliphatic (e.g., C₁₋ ₆alkyl). Acceptable esters also include cycloalkyl esters and arylalkyl esters such as, but not limited to, benzyl. Examples of pharmaceutically acceptable amides of the compound embodiments of this disclosure include, but are not limited to, primary amides, and secondary and tertiary alkyl amides (for example with between one and six carbons). Amides and esters of disclosed exemplary embodiments of compound embodiments according to the present disclosure can be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference.

Retina: The light (photon) sensitive portion of the eye, that contains the photoreceptors (cones and rods) for light. The layers of the retina include a) membrana limitans interna; b) stratum opticum, c) ganglionic layer; d) inner plexiform layer; e) inner nuclear layer; f) outer plexiform layer; g) outer nuclear layer; h) membrana limtans externa; i) layer of rods and cones (inner and outer segments of photoreceptors), and j) the retinal pigment epithelium. In adult wild-type (non-diseased) humans, the entire retina is approximately 72% of a sphere about 22 mm in diameter. The entire retina contains about 7 million cones and 75 to 150 million rods. Rods and cones perform light perception through the use of light sensitive pigments, which are phototransduction proteins that initiate visual cycle, and thus the rods and cones are vison-forming photoreceptors. The light sensitive pigments include proteins called opsins and a chromophore called retinal, which is the variant of vitamin A. The rods contain rhodopsin while the cones contain cone opsins such as S-opsin, M-opsin and L-opsin. Rods and cones transmit signals through successive neurons that trigger a neural discharge in the output cells of the retina and the ganglion cells. The visual signals are conveyed by the optic nerve to the lateral geniculate bodies from where the visual signal is passed to the visual cortex (occipital lobe) and registered as a visual stimulus. “Rod cells”, or “rods,” are photoreceptor cells in the retina of the eye that can function in less intense light than the other type of visual photoreceptor, cone cells. Rods are concentrated at the outer edges of the retina and are used in peripheral vision. Rods are a little longer and leaner than cones but have the same structural basis. The opsin or pigment is on the outer side, adjacent to retinal pigment epithelium, completing the cell’s homeostasis. This epithelium end contains many stacked disks. Rods have a high area for visual pigment and thus substantial efficiency of light absorption. Like cones, rod cells have a synaptic terminal, an inner segment, and an outer segment. The synaptic terminal forms a synapse with another neuron, for example a bipolar cell. The inner and outer segments are connected by the connecting cilium, which lines the distal segment. The inner segment contains organelles and the cell’s nucleus, while the rod outer segment, which is pointed toward the back of the eye, contains the light-absorbing materials. Activation of photopigments by light sends a signal by hyperpolarizing the rod cell, leading to the rod cell not sending its neurotransmitter, which leads to the bipolar cell then releasing its transmitter at the bipolar-ganglion synapse and exciting the synapse. “Cone cells,” or “cones,” are photoreceptor responsible for color vision and function best in relatively bright light. Cone cells are densely packed in the fovea centralis, a 0.3 mm diameter rod-free area with very thin, densely packed cones which quickly reduce in number towards the periphery of the retina. There are about six to seven million cones in a human eye and are most concentrated towards the macula. Cones are less sensitive to light than the rod cells in the retina (which support vision at low light levels) but allow the perception of color. They are also able to perceive finer detail and more rapid changes in images, because their response times to stimuli are faster than those of rods. In humans, cones are normally one of the three types, each with different pigment, namely: S-cones, M-cones and L-cones. Each cone is therefore sensitive to visible wavelengths of light that correspond to short-wavelength, medium-wavelength and long-wavelength light. The three types have peak wavelengths near 420-440 nm, 534-545 nm and 564-580 nm, respectively, depending on the individual.

Retinal Pigment Epithelium: The pigmented layer of hexagonal cells, present in vivo in mammals, just outside of the neurosensory retinal that is attached to the underlying choroid. These cells are densely packed with pigment granules and shield the retinal from incoming light. The retinal pigment epithelium also serves as the limiting transport factor that maintains the retinal environment by supplying small molecules such as amino acid, ascorbic acid and D-glucose while remaining a tight barrier to choroidal blood borne substances.

Retinitis pigmentosa (RP): An inherited, degenerative eye disease that causes severe vision impairment due to the progressive degeneration of the rod photoreceptor cells in the retina. This form of retinal dystrophy manifests initial symptoms independent of age. The initial retinal degenerative symptoms of Retinitis pigmentosa are characterized by decreased night vision (nyctalopia) and the loss of the mid-peripheral visual field. The rod photoreceptor cells, which are responsible for low-light vision and are orientated in the retinal periphery, are the retinal processes affected first during non-syndromic forms of this disease. Visual decline progresses relatively quickly to the far peripheral field, eventually extending into the central visual field as tunnel vision increases. Visual acuity and color vision can become compromised due to accompanying abnormalities in the cone photoreceptor cells, which are responsible for color vision, visual acuity, and sight in the central visual field. The progression of disease symptoms occurs in a symmetrical manner, with both the left and right eyes experiencing symptoms at a similar rate. There are multiple genes that, when mutated, can cause the retinitis pigmentosa phenotype. Inheritance patterns of RP have been identified as autosomal dominant, autosomal recessive, X-linked, and maternally (mitochondrially) acquired, and are dependent on the specific RP gene mutations present in the parental generation.

Rhodopsin: A light-sensitive G-protein coupled receptor protein involved in visual phototransduction. Rhodopsin consists of two components, a protein molecule also called rod visual opsin and a covalently-bound cofactor called retinal. Rod visual opsin is an opsin, a light-sensitive G-protein coupled receptor that embeds in the lipid bilayer of cell membranes using seven protein transmembrane domains. These domains form a pocket where the photoreactive chromophore, retinal, lies horizontally to the cell membrane, linked to a lysine residue in the seventh transmembrane domain of the protein. Thousands of rhodopsin molecules are found in each outer segment disc of the host rod cell. Retinal is produced in the retina from vitamin A, from dietary beta-carotene. Isomerization of 11-cis-retinal into all-trans-retinal by light sets off a series of conformational changes (‘bleaching’) in the opsin, eventually leading it to a form called metarhodopsin II (Meta II), which activates an associated G protein, transducin, to trigger a cyclic guanosine monophosphate (cGMP) second messenger cascade. An exemplary human rhodopsin protein sequence is disclosed in GENBANK® Accession No. NP_000530, as available on Jun. 1, 2020, incorporated herein by reference, and an exemplary mRNA encoding human rhodopsin is disclosed in GENBANK® Accession No. NM_000539, as available on Jun. 1, 2020, incorporated herein by reference.

Rod cyclic GMP phosphodiesterase 6β (PDE6β): The beta subunit of the protein complex PDE6 that is encoded by the PDE6B gene. PDE6 is crucial in transmission and amplification of visual signal; mutations in this subunit are responsible for retinal degermation, such as in retinitis pigmentosa or congenital statutory night blindness. Exemplary human orthologs are disclosed, for example, in GENBANK® Accession Nos. NM_000283 mRNA) and NP_000274 (protein), as available on Jun. 1, 2020, incorporated herein by reference.

Silyl Ether: -OSiR^{a}R^{b}, wherein each of R^{a} and R^{b} independently is selected from hydrogen, aliphatic, heteroaliphatic, haloaliphatic, haloheteroaliphatic, aromatic, or an organic functional group.

Stargardt Disease: A disease also known as juvenile macular degeneration, that is the most common single-gene inherited retinal disease. It usually has an autosomal recessive inheritance caused by mutations in the ABCA4 gene. Rarely it has an autosomal dominant inheritance due to defects with ELOVL4 or PROM1 genes. It is characterized by macular degeneration that begins in childhood, adolescence or adulthood, resulting in progressive loss of vision.

Presentation usually occurs in childhood or adolescence, though there is no upper age limit for presentation. The main symptom is loss of visual acuity, uncorrectable with glasses. The vision loss usually manifests as the loss of the ability to see fine details when reading or seeing distant objects. Symptoms typically develop before age 20 (median age of onset: ~17 years old), and include: wavy vision, blind spots, blurriness, loss of depth perception, sensitivity to glare, impaired color vision, and difficulty adapting to dim lighting (delayed dark adaptation). Peripheral vision is usually less affected than fine, central (foveal) vision.

Subject: Mammals and other animals, such as humans, companion animals (e.g., dogs, cats, rabbits, etc.), utility animals, and feed animals; thus, disclosed methods are applicable to both human therapy and veterinary applications.

Sulfinyl: -S(O)R^{a}, wherein R^{a} is selected from hydrogen, aliphatic, heteroaliphatic, haloaliphatic, haloheteroaliphatic, aromatic, or an organic functional group.

Sulfonyl: -SO₂R^{a}, wherein R^{a} is selected from hydrogen, aliphatic, heteroaliphatic, haloaliphatic, haloheteroaliphatic, aromatic, or an organic functional group.

Sulfonamide: -SO₂NR^{a}R^{b} or -N(R^{a})SO₂R^{b}, wherein each of R^{a} and R^{b} independently is selected from hydrogen, aliphatic, heteroaliphatic, haloaliphatic, haloheteroaliphatic, aromatic, or an organic functional group.

Sulfonate: —SO₃⁻, wherein the negative charge of the sulfonate group may be balanced with an M⁺ counter ion, wherein M⁺ may be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Therapeutically Effective Amount: An amount of a compound sufficient to treat a specified disorder or disease, or to ameliorate or eradicate one or more of its symptoms and/or to prevent the occurrence of the disease or disorder, such as retinal degeneration. The amount of a compound which constitutes a “therapeutically effective amount” will vary depending on the compound, the disease state and its severity, the age of the subject to be treated, and the like. The therapeutically effective amount can be determined by a person of ordinary skill in the art.

Thial: —C(S)H.

Thioacyl: -C(S)R^{a} wherein R^{a} is selected from hydrogen, aliphatic, heteroaliphatic, haloaliphatic, haloheteroaliphatic, aromatic, or an organic functional group.

Thiocarboxylic acid: —C(O)SH, or —C(S)OH.

Thiocyanate: —S—CN or —N═C═S.

Thioester: -C(O)SR^{a} or -C(S)OR^{a} wherein R^{a} is selected from hydrogen, aliphatic, heteroaliphatic, haloaliphatic, haloheteroaliphatic, aromatic, or an organic functional group.

Thioether: -S-aliphatic or -S-aromatic, such as -S-alkyl, -S-alkenyl, -S-alkynyl, -S-aryl, or -S-heteroaryl; or -aliphatic-S-aliphatic, -aliphatic-S-aromatic, -aromatic-S-aliphatic, or -aromatic-S-aromatic.

Treating, Treatment, and Therapy: Any success or indicia of success in the attenuation or amelioration of an injury, pathology or condition, including any objective or subjective parameter such as abatement, remission, diminishing of symptoms or making the condition more tolerable to the subject, slowing in the rate of degeneration or decline, making the final point of degeneration less debilitating, improving a subject’s physical or mental well-being, or improving vision. The treatment may be assessed by objective or subjective parameters; including the results of a physical examination, neurological examination, or psychiatric evaluations. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease, such as improved vision. A “prophylactic” treatment is a treatment administered to a subject who does not exhibit signs of a disease or exhibits only early signs for the purpose of decreasing the risk of developing pathology.

As used herein, the terms “disease” and “condition” can be used interchangeably or can be different in that the particular malady or condition may not have a known causative agent (so that etiology has not yet been determined) and it is therefore not yet recognized as a disease but only as an undesirable condition or syndrome, where a more or less specific set of symptoms have been identified by clinicians.

### II. Introduction

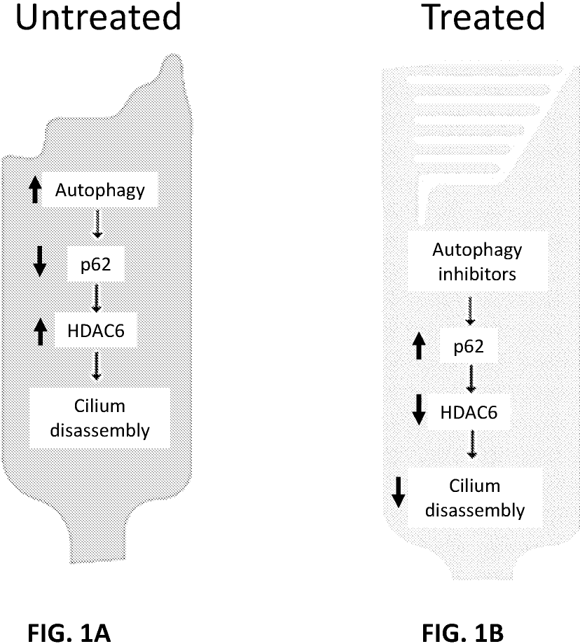

Disclosed herein are compounds for treating and/or preventing retinal degeneration in a subject. In some embodiments, the compounds are able to maintain photoreceptor survival. For example, in some embodiments, the compounds improve expression and polarity of rhodopsin and/or increase S-opsin expression and polarization in cone photoreceptors. In yet additional embodiments, the compounds are able to increase ciliary proteins in photoreceptors. In some embodiments, the compounds are autophagy inhibitors and can reduce the autophagy pathway, thus improving cilium biogenesis and maintaining photoreceptor survival in retinal degenerative diseases. In retinal ciliopathies, autophagy pathway is activated by the stress resulting from ciliary defects (e.g., mislocalization of the outer segment proteins in the inner segment). Subsequently, p62 levels decrease due to acceleration of protein degradation, leading to an increase of its interaction partner in photoreceptor HDAC6, a driver of cilium disassembly (FIG. 1A). Compound embodiments of the present disclosure inhibit the fusion of the autophagosome with the lysosome, thereby increasing p62 and restoring the degradation of HDAC6 (FIG. 1B). With a reduced autophagy pathway and subsequent improvement of cilium biogenesis, the compound embodiments can maintain photoreceptor survival in retinal degenerative diseases, such as (but not limited to) LCA or retinitis pigmentosa.

Method embodiments also are disclosed for treating retinal degeneration in a subject in need thereof. In some embodiments, the method comprises administering to the subject a therapeutically effective amount of 3-(dibutylamino)-1-(1,3-dichloro-6-(trifluoromethyl)phenanthren-9-yl)propan-1-ol hydrochloride or a compound having a structure according to a formula selected from Formula I, II, or III, as provided herein. In some non-limiting examples, the subject has retinitis pigmentosa, LCA, Stargardt’s macular dystrophy, cone-rod dystrophy, choroideremia or age-related macular degeneration. Additional compound embodiments are described herein, including pharmaceutically acceptable salts, prodrugs, solvates, hydrates, and/or tautomers thereof, along with composition embodiments comprising the same.

### III. Compound Embodiments

Compound embodiments of the present disclosure can have a structure according to any one of Formulas I-III, illustrated below, including a pharmaceutically acceptable salt, prodrug, solvate, hydrate, or tautomer thereof.

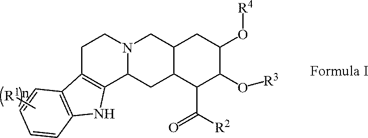

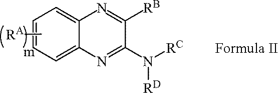

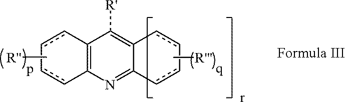

With reference to Formula I, the following variable recitations can apply:
R¹, if present (such as when n is an integer other than 0), can be heteroaliphatic;
R² can be selected from OR⁵, or NR⁶R⁷, wherein each of R⁵, R⁶, and R⁷ independently is selected from hydrogen, aliphatic, or aromatic, or an organic functional group;
each of R³ and R⁴ independently can be selected from aliphatic, aromatic, acyl, or sulfonyl; and
n can be an integer selected from 0 to 4.

In embodiments of Formula I when n is 0, a person of ordinary skill in the art will recognize that a hydrogen atom is present to satisfy the valency of any of the carbon atom(s) that would otherwise be bound to any R¹ group.

With reference to Formula II, the following variable recitations can apply:
R^{A}, if present (such as when m is an integer other than 0), can be selected from halogen, heteroaliphatic, haloaliphatic, or an organic functional group;
R^{B} can be aromatic; and
each of R^{C} and R^{D} independently can be selected from hydrogen, aliphatic, or heteroaliphatic, or R^{C} and R^{D} can join together to form a heterocylic ring system along with the nitrogen atom to which they are bound; and
m can be an integer selected from 0 to 4.

In embodiments of Formula II when m is 0, a person of ordinary skill in the art will recognize that a hydrogen atom is present to satisfy the valency of any of the carbon atom(s) that would otherwise be bound to any R^{A} group.

With reference to Formula III, the following variable recitations can apply:
R′, if present (such as when the optional bond represented by the dashed line is present), can be selected from aliphatic, aromatic, halogen, heteroaliphatic, haloaliphatic, or an organic functional group;
each R″, if present (such as when p is an integer other than 0), independently can be selected from halogen, heteroaliphatic, or amino;
each R‴, if present (such as when q is an integer other than 0 and r is 1), independently can be selected from halogen, heteroaliphatic, or amino;
p is an integer selected from 0 to 4;
q is an integer selected from 0 to 4; and
r is an integer selected from 0 or 1.

In embodiments of Formula III when p and/or q is 0, a person of ordinary skill in the art will recognize that a hydrogen atom is present to satisfy the valency of any of the carbon atom(s) that would otherwise be bound to the any R′ or R″ group, respectively. A person of ordinary skill in the art also would further recognize that if r is 0, then the valency of each of the carbon atoms to which the fused ring would otherwise be attached is satisfied by a hydrogen atom. Also, a person of ordinary skill in the art will recognize that if any of the optional bonds represented by dashed lines in Formula III are not present, then the valency of the corresponding carbon atom(s) of the formula is/are satisfied by a bond to a hydrogen atom.

In particular embodiments of Formula I (and including any Formulas IA-IE below), the following variable recitations can apply:
R¹ can be alkoxy, such as —OMe, —OEt, —OPr, —OiPr, —OnBu, —OₜBu, and the like;
R² can be -OR⁵ or -NR⁶R⁷, wherein each of R⁵, R⁶, and R⁷ independently can be selected from hydrogen, alkyl (e.g., C₁₋₆alkyl or C₃-C₆cycloalkyl), heteroaryl (e.g., C₃₋₆heteroaryl), or aryl (e.g., C₆₋₁₀aryl);
each of R³ and R⁴ independently can be selected from alkyl (e.g., C₁₋₆alkyl or C₃₋₆cycloalkyl); heteroaryl (e.g., C₃₋₁₀heteroaryl); aryl (e.g., C₆₋₁₀aryl); or an organic functional group selected from sulfonyl or acyl. In some embodiments, the sulfonyl group can have a formula -SO₂R⁹, wherein R⁹ can be selected from aliphatic, amine, or aromatic. In some embodiments, the acyl group can have a formula -C(O)R⁸, wherein R⁸ can be selected from alkyl (e.g., C₁₋₁₀alkyl, C₁₋₁₀cycloalkyl, C₁₋₁₀alkenyl, or C₁₋₁₀cycloalkenyl); heteroaryl (e.g., C₄₋₁₀heteroaryl); heteroaryl (e.g., C₄₋₁₀heteroaryl) comprising one or more substituents selected from halogen, —CF₃, —CN, —OH, C₁₋₆alkyl, C₁₋₆alkoxy, sulfonyl, sulfonamide (e.g., C₁₋₆alkylsulfonylamino, such as -SO₂NHC₁₋₆alkyl), amide (e.g., C₁₋₆alkylaminocarbonyl, such as -C(O)NHC₁₋₆alkyl); aryl (e.g., C₆₋₁₀aryl); aryl (e.g., C₆₋₁₀aryl) comprising one or more substituents selected from halogen, —CF₃, —CN, —OH, C₁₋₆alkyl, C₁₋₆alkoxy, sulfonyl, sulfonamide (e.g., C₁₋₆alkylsulfonylamino, such as -SO₂NHC₁₋₆alkyl), amide (e.g., C₁₋₆alkylaminocarbonyl, such as -C(O)NHC₁₋₆alkyl); and
n is 0, 1, 2, 3, or 4.

In some embodiments of Formula I, the compound can have the stereochemistry illustrated below in Formula IA, including a pharmaceutically acceptable salt, prodrug, solvate, hydrate, or tautomer thereof.

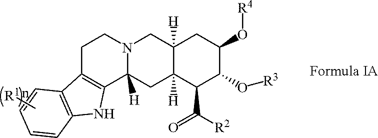

In some embodiments, the compound embodiments of Formula I and/or IA can further have structure according to Formula IB, IC, ID, or IE, including a pharmaceutically acceptable salt, prodrug, solvate, hydrate, or tautomer thereof.

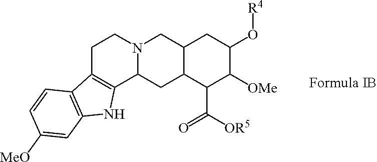

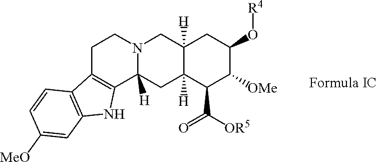

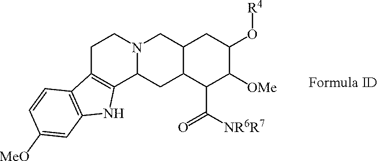

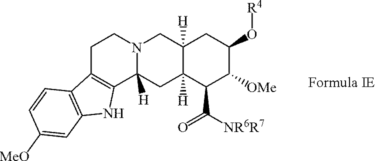

In some additional embodiments of any of Formulas I or IA-IE, R⁴ can be selected from any of the following groups:

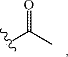

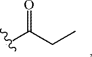

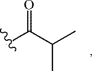

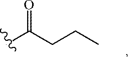

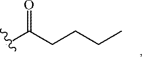

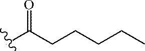

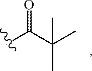

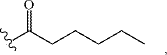

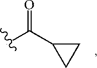

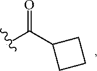

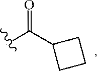

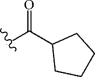

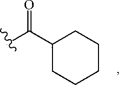

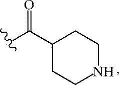

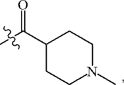

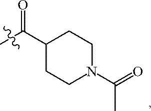

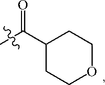

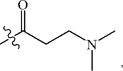

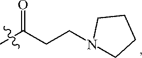

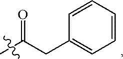

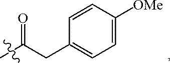

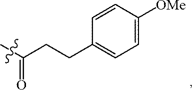

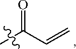

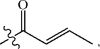

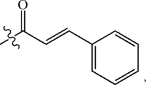

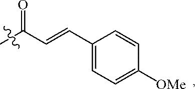

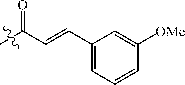

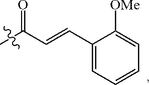

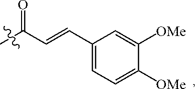

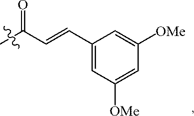

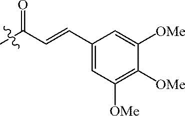

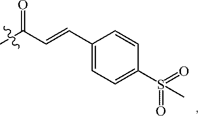

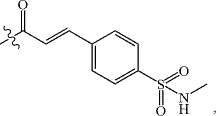

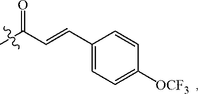

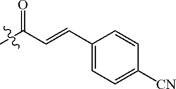

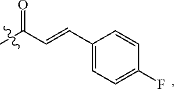

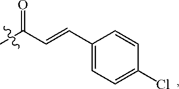

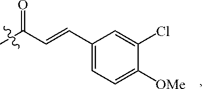

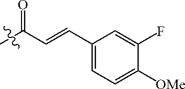

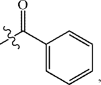

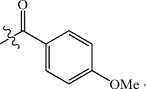

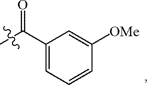

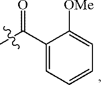

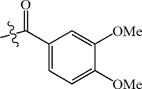

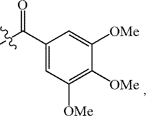

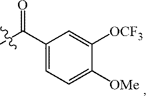

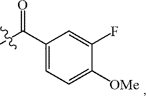

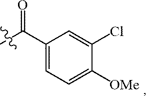

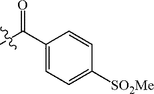

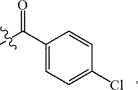

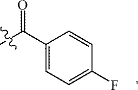

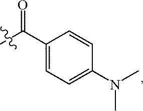

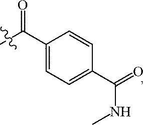

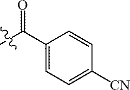

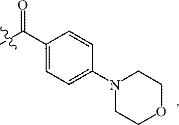

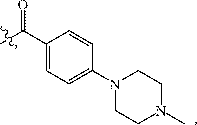

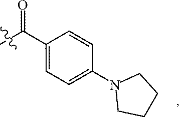

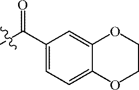

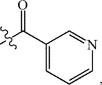

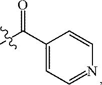

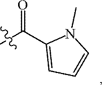

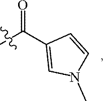

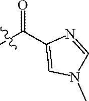

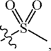

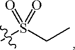

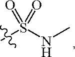

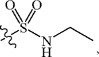

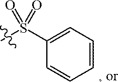

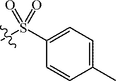

In representative embodiments, both R¹ and R² are —OMe, R³ is methyl, and R⁴ is selected from any of the groups illustrated above. In particular embodiments, compounds of Formula I are selected from any of the following, including any pharmaceutically acceptable salt, prodrug, solvate, hydrate, or tautomer thereof.

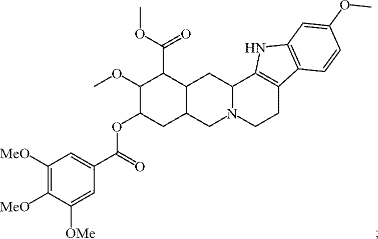

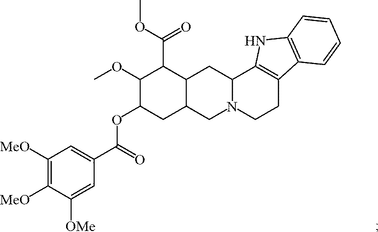

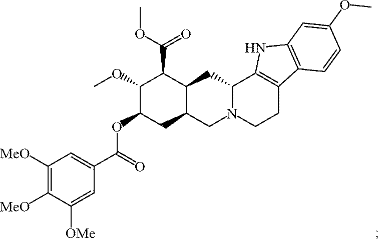

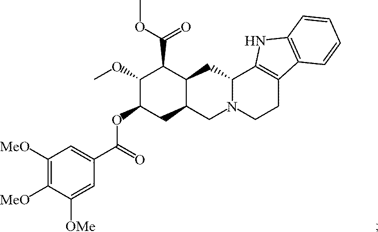

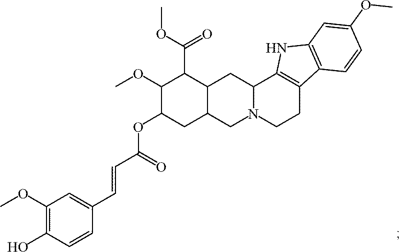

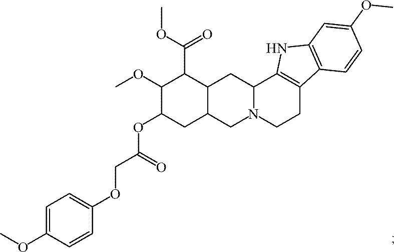

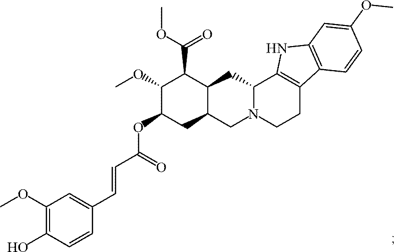

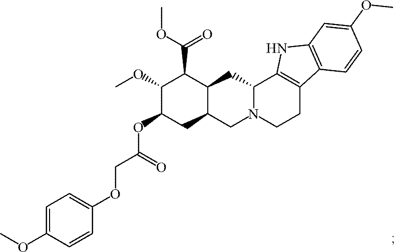

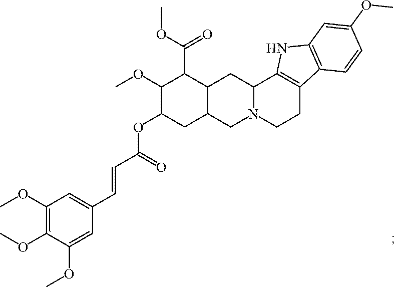

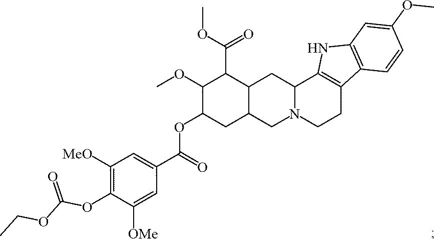

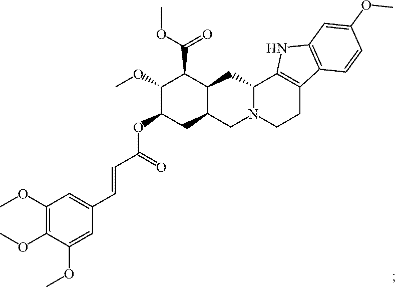

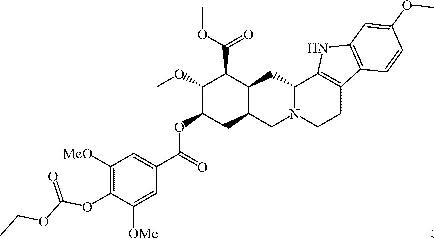

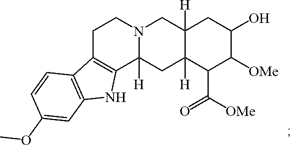

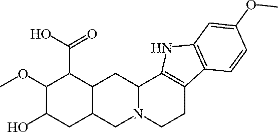

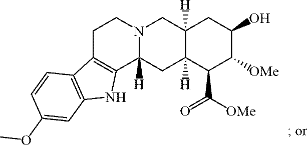

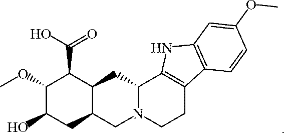

In some embodiments, the compound can be selected from any of the following, including a pharmaceutically acceptable salt, prodrug, solvate, hydrate, or tautomer thereof:
methyl (1S,2R,3R,4aS,13bR,14aS)-2,11-dimethoxy-3-((3,4,5-trimethoxybenzoyl)oxy)-1,2,3,4,4a,5,7,8,13,13b,14,14a-dodecahydroind olo[2′,3′:3 ,4]pyrido[1,2-b]isoquinoline-1-carboxylate (also referred to herein as “Reserpine” or “NCGC0091250”);
methyl (1S,2R,3R,4aS,13bR,14aS)-2-methoxy-3-((3,4,5-trimethoxybenzoyl)oxy)-1,2,3,4,4a,5,7,8,13,13b,14,14a-dodecahydroind olo[2′,3′:3 ,4]pyrido[1,2-b]isoquinoline-1-carboxylate;
methyl (1S,2R,3R,4aS,13bR,14aS)-3-(((E)-3-(4-hydroxy-3-methoxyphenyl)acryloyl)oxy)-2,11-dimethoxy-1,2,3,4,4a,5,7,8,13,13b,14,14a-dodecahydroind olo[2′,3′:3 ,4]pyrido[1,2-b]isoquinoline-1-carboxylate (also referred to herein as “Rescimetol” or “NCGC00253604”);
methyl (1S,2R,3R,4aS,13bR,14aS)-2,11-dimethoxy-3-(2-(4-methoxyphenoxy)acetoxy)-1,2,3,4,4a,5,7,8,13,13b,14,14a-dodecahydroind olo[2′,3′:3 ,4]pyrido[1,2-b]isoquinoline-1-carboxylate;
methyl (1S,2R,3R,4aS,13bR,14aS)-2,11-dimethoxy-3-(((E)-3-(3,4,5-trimethoxyphenyl)acryloyl)oxy)-1,2,3,4,4a,5,7,8,13,13b,14,14a-dodecahydroind olo[2′,3′:3 ,4]pyrido[1,2-b]isoquinoline-1-carboxylate;
methyl (1S,2R,3R,4aS,13bR,14aS)-3-((4-((ethoxycarbonyl)oxy)-3,5-dimethoxybenzoyl)oxy)-2,11-dimethoxy-1,2,3,4,4a,5,7,8,13,13b,14,14a-dodecahydroind olo[2′,3′:3 ,4]pyrido[1,2-b]isoquinoline-1-carboxylate;
methyl (1S,2R,3R,4aS,13bR,14aS)-3-hydroxy-2,11-dimethoxy-1,2,3,4,4a,5,7,8,13,13b,14,14a-dodecahydroind olo[2′,3′:3 ,4]pyrido[1,2-b]isoquinoline-1-carboxylate;
(1S,2R,3R,4aS,13bR,14aS)-3-hydroxy-2,11-dimethoxy-1,2,3,4,4a,5,7,8,13,13b,14,14a-dodecahydroind olo[2′,3′:3 ,4]pyrido[1,2-b]isoquinoline-1-carboxylic acid;
methyl 2,11-dimethoxy-3-((3,4,5-trimethoxybenzoyl)oxy)-1,2,3,4,4a,5,7,8,13,13b,14,14a-dodecahydroind olo[2′,3′:3 ,4]pyrido[1,2-b]isoquinoline-1-carboxylate;
methyl 2-methoxy-3-((3,4,5-trimethoxybenzoyl)oxy)-1,2,3,4,4a,5,7,8,13,13b,14,14a-dodecahydroind olo[2′,3′:3 ,4]pyrido[1,2-b]isoquinoline-1-carboxylate;
methyl (E)-3-((3-(4-hydroxy-3-methoxyphenyl)acryloyl)oxy)-2,11-dimethoxy-1,2,3,4,4a,5,7,8,13,13b,14,14a-dodecahydroind olo[2′,3′:3 ,4]pyrido[1,2-b]isoquinoline-1-carboxylate;
methyl 2,11-dimethoxy-3-(2-(4-methoxyphenoxy)acetoxy)-1,2,3,4,4a,5,7,8,13,13b,14,14a-dodecahydroind olo[2′,3′:3 ,4]pyrido[1,2-b]isoquinoline-1-carboxylate;
methyl (E)-2,11-dimethoxy-3-((3-(3,4,5-trimethoxyphenyl)acryloyl)oxy)-1,2,3,4,4a,5,7,8,13,13b,14,14a-dodecahydroind olo[2′,3′:3 ,4]pyrido[1,2-b]isoquinoline-1-carboxylate;
methyl 3-((4-((ethoxycarbonyl)oxy)-3,5-dimethoxybenzoyl)oxy)-2,11-dimethoxy-1,2,3,4,4a,5,7,8,13,13b,14,14a-dodecahydroind olo[2′,3′:3 ,4]pyrido[1,2-b]isoquinoline-1-carboxylate;
methyl 3-hydroxy-2,11-dimethoxy-1,2,3,4,4a,5,7,8,13,13b,14,14a-dodecahydroind olo[2′,3′:3 ,4]pyrido[1,2-b]isoquinoline-1-carboxylate; or
3-hydroxy-2,11-dimethoxy-1,2,3,4,4a,5,7,8,13,13b,14,14a-dodecahydroind olo[2′,3′:3 ,4]pyrido[1,2-b]isoquinoline-1-carboxylic acid.

In particular embodiments of Formula II (or Formula IIA, below), the following variable recitations can apply:
each R^{A} independently can be selected from halogen (e.g., Cl, F, Br, or I), —OMe, —CN, or —CF₃;
R^{B} can be selected from aryl (e.g., C₆₋₁₀aryl); aryl (e.g., C₆₋₁₀aryl) comprising one or more substituents selected from halogen, —CF₃, —CN, —OH, alkyl (e.g., C₁₋₆ alkyl), alkoxy (e.g., C₁₋₆alkoxy); heteroaryl (e.g., C₄₋₁₀heteroaryl); heteroaryl (e.g., C₄₋₁₀heteroaryl) comprising one or more substituents selected from halogen, —CF₃, —CN, —OH, alkyl (e.g., C₁₋₆ alkyl), alkoxy (e.g., C₁₋₆alkoxy);
each of R^{C} and R^{D} independently can be selected from hydrogen, alkyl (e.g., C₁₋₁₂alkyl or C₃₋₈cycloalkyl), amino (e.g., C₁₋₁₂alkylaminoalkyl, such as N,N-diethylaminobutanyl; or C₃₋₈cycloalkylaminoalkyl), or R^{C} and R^{D} can join together to form a four-, five-, six-, or seven-membered heterocylic ring system, including aromatic and non-aromatic versions thereof, along with the nitrogen atom to which they are bound; and
m is 0, 1, 2, 3, or 4.

In some embodiments, the compound embodiments of Formula II can further have a structure according to Formula IIA, including a pharmaceutically acceptable salt, prodrug, solvate, hydrate, or tautomer thereof.

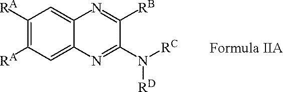

In some additional embodiments of any of Formulas II or IIA, R^{B} can be selected from any of the following groups:

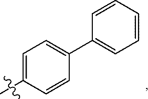

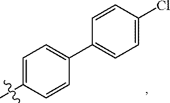

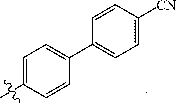

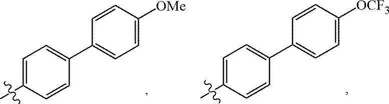

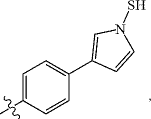

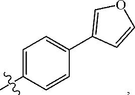

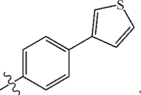

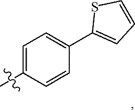

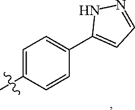

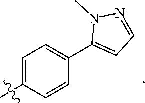

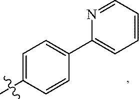

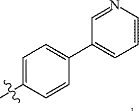

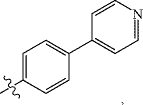

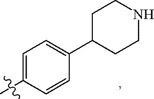

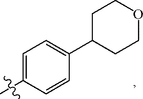

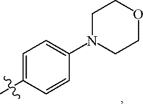

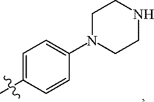

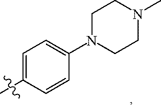

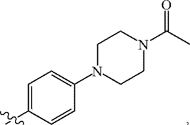

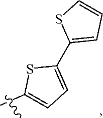

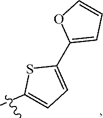

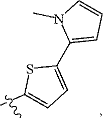

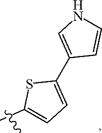

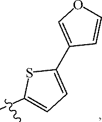

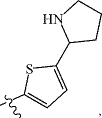

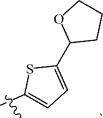

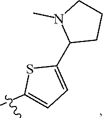

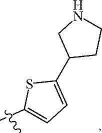

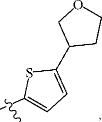

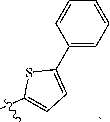

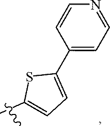

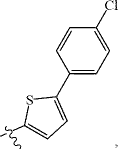

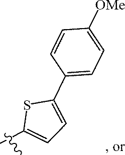

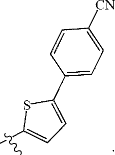

In representative embodiments, m is 2 and each R^{A} independently is a halogen (e.g., Cl), R^{B} is not present or is a bi-thiophene group, and one of R^{C} and R^{D} is hydrogen and the other is N,N-diethylaminobutanyl. In particular embodiments, the compound is

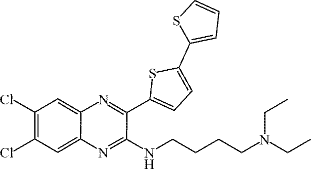 [SMILES: CCN(CC)CCCCNc1nc2cc(Cl)c(Cl)cc2nc1-c1ccc(-c2cccs2)s1]

N1-(3-([2,2′-bithiophen]-5-yl)-6,7-dichloroquinoxalin-2-yl)-N4,N4-diethylbutane-1,4-diarnine (also referred to herein as “NCGC00263128”) or any pharmaceutically acceptable salt, prodrug, solvate, hydrate, or tautomer thereof.

In particular embodiments of Formula III (or any one of Formulas IIIA-IIID), the following variable recitations can apply:
R′, when present, can be selected from halo, —CN, —CF₃, —OCF₃, alkyl (e.g., C₁₋₁₂alkyl), heteroalkyl (e.g., C₁₋₁₂heteroalkyl comprising one or more nitrogen atoms, one or more oxygen atoms, one or more sulfur atoms, or a combination thereof and including cyclic and acyclic versions thereof), aminoaryl (e.g., -NR^{a′}-aryl or -NR^{a′}-aryl comprising one or more substituents selected from halogen, —CN, —CF₃, —OCF₃, amino, heteroalkyl, amide, or sulfonamide);
R″, when present, can be selected from halogen, alkoxy, or -NR^{a′}R^{b′}, herein each of R^{a′} and R^{b′} independently is selected from alkyl (e.g., C₁₋₁₂alkyl), heteroalkyl (e.g., C₁₋₁₂heteroalkyl), benzyl (e.g., -CH₂aromatic or -CH₂aromatic comprising one or more substituents selected from alkoxy, halogen, or amide), acyl (e.g., -C(O)alkyl, -C(O)alkenyl, -C(O)heteroalkyl, -C(O)aromatic, -C(O)aromatic comprising one or more substituents selected from alkyl, halogen, —CF₃, —OCF₃, or —CN), sulfonyl (e.g., —SO₂R^{a}, wherein R^{a} is selected from hydrogen, alkyl, aromatic, aromatic comprising one or more substituents selected from alkyl, amide, or alkoxy);
R‴, when present, can be selected from halogen, alkoxy, or -NR^{a′}R^{b′}, wherein each of R^{a′} and R^{b′} independently is selected from alkyl (e.g., C₁₋₁₂alkyl), heteroalkyl (e.g., C₁₋₁₂heteroalkyl), benzyl (e.g., -CH₂aromatic or -CH₂aromatic comprising one or more substituents selected from alkoxy, halogen, or amide), acyl (e.g., -C(O)alkyl, -C(O)alkenyl, -C(O)heteralkyl, -C(O)aromatic, -C(O)aromatic comprising one or more substituents selected from alkyl, halogen, —CF₃, —OCF₃, or —CN), sulfonyl (e.g., —SO₂R^{a}, wherein R^{a} is selected from hydrogen, alkyl, aromatic, aromatic comprising one or more substituents selected from alkyl, amide, or alkoxy);
each of p and q independently is an integer selected from 0, 1, 2, 3, or 4; and
r is 0 or 1.

In some additional embodiments, R′ is present, R″ is present, and R‴ is not present and r is 0. In some other embodiments, each of R′, R″, and R‴ is present and R″ and R‴ are the same. In yet some additional embodiments, each of R″ and R‴ is present and R′ is not present. In yet some additional embodiments, R′ is present and neither of R″ or R‴ is present and in some such embodiments, r can be 1 and the resulting ring can be saturated. In particular embodiments, each of R″ and R‴ can be the same or different. Exemplary formulas illustrated at least certain of these options are illustrated below.

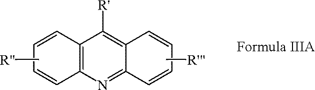

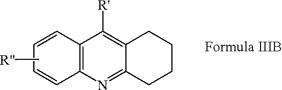

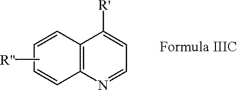

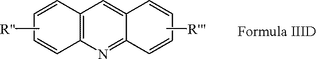

In some additional embodiments of any of Formulas IIIA-IIID, R′ can be selected from any of the following groups:

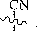

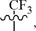

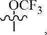

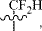

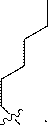

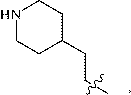

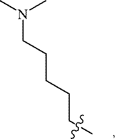

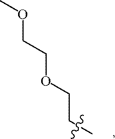

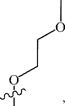

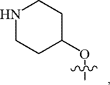

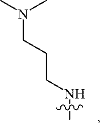

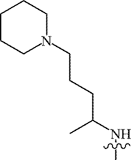

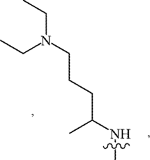

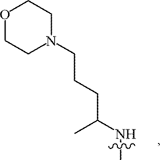

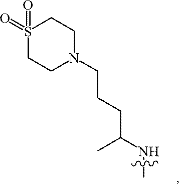

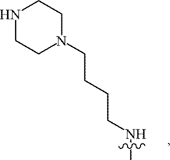

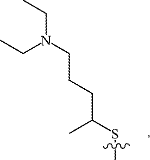

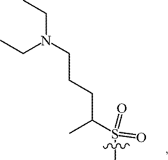

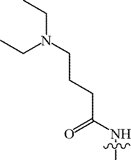

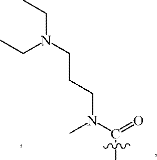

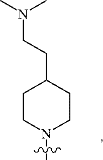

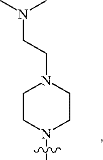

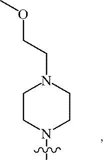

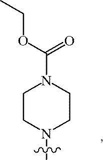

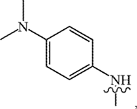

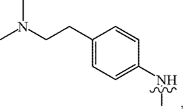

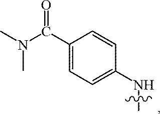

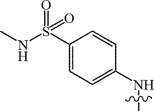

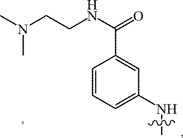

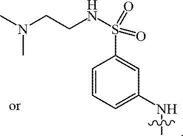

In embodiments wherein R″ and/or R‴ is -NR^{a′}R^{b′}, each of R^{a′} and/or R^{b′} can be selected from any of the following groups, as well as hydrogen:

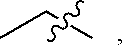

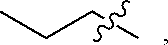

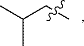

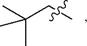

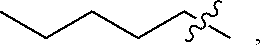

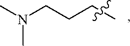

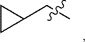

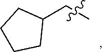

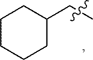

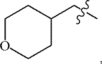

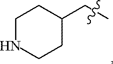

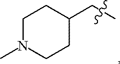

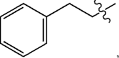

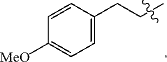

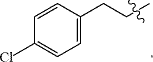

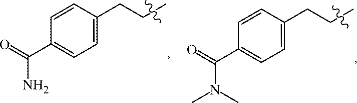

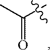

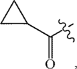

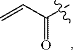

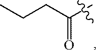

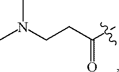

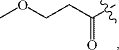

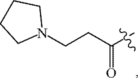

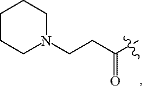

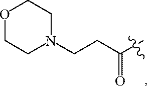

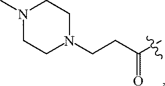

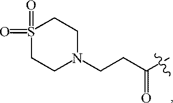

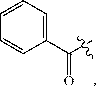

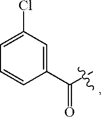

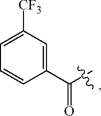

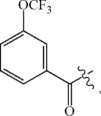

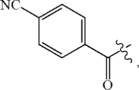

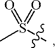

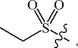

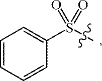

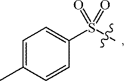

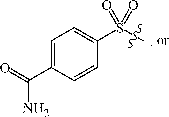

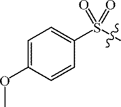

In representative embodiments, R′ is heteroalkyl, R″ and R‴ are present and are Cl and OMe, respectively. In particular embodiments, compounds of Formula III are selected from the following, including any pharmaceutically acceptable salt, prodrug, solvate, hydrate, or tautomer thereof.

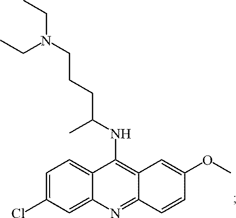

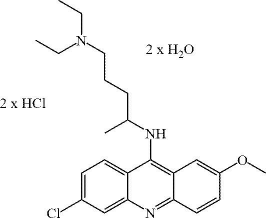

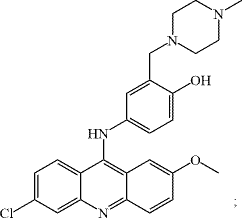

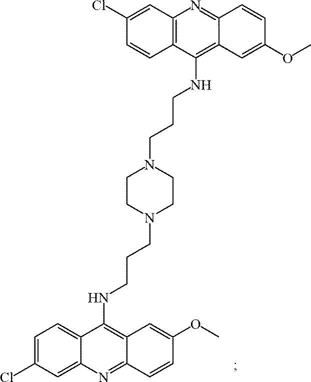

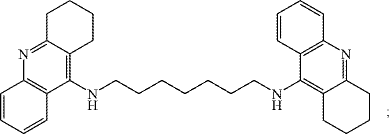

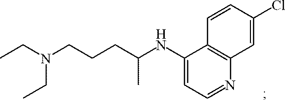

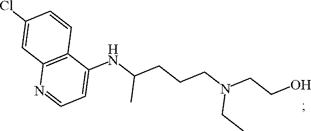

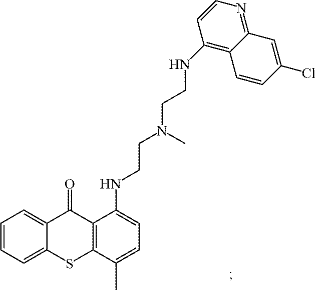

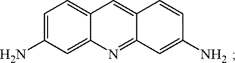

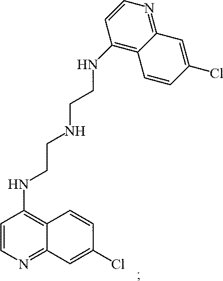

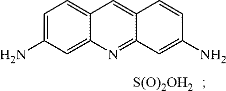

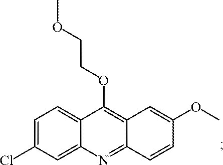

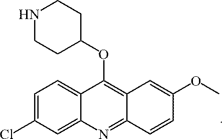

In some embodiments, the compound can be selected from any of the following:
N4-(6-chloro-2-methoxyacridin-9-yl)-N1,N1-diethylpentane-1,4-diamine;
N,N′-(piperazine-1,4-diylbis(propane-3,1-diyl))bis(6-chloro-2-methoxyacridin-9-amine);
N4-(6-chloro-2-methoxyacridin-9-yl)-N1,N1-diethylpentane-1,4-diamine dihydrochloride dihydrate (also referred to herein as “Quinacrine dihydrochloride dihydrate” or “NCGC0015874”);
N,N′-(piperazine-1,4-diylbis(propane-3,1-diyl))bis(6-chloro-2-methoxyacridin-9-amine);
N1,N7-bis(1,2,3,4-tetrahydroacridin-9-yl)heptane-1,7-diamine;
N4-(7-chloroquinolin-4-yl)-N1,N1-diethylpentane-1,4-diamine (also referred to herein as “chloroquine”);
2-((4-((7-chloroquinolin-4-yl)amino)pentyl)(ethyl)amino)ethan-1-ol (also referred to herein as “hydroxychloroquine”);
1-((2-((2-((7-chloroquinolin-4-yl)amino)ethyl)(methyl)amino)ethyl)amino)-4-methyl-9H-thioxanthen-9-one (also referred to herein as “ROC-325”);
acridine-3,6-diamine;
acridine-3,6-diamine hemisulfate (also referred to herein as “Proflavine hemisulfate”);
6-chloro-2-methoxy-9-(2-methoxyethoxy)acridine;
N1-(7-chloroquinolin-4-yl)-N2-(2-((7-chloroquinolin-4-yl)amino)ethyl)ethane-1,2-diamine (also referred to herein as “Lys05”); or
6-chloro-2-methoxy-9-(piperidin-4-yloxy)acridine.

In an independent embodiment, the compound can be 3-(dibutylamino)-1-(1,3-dichloro-6-(trifluoromethyl)phenanthren-9-yl)propan-1-ol hydrochloride (or a pharmaceutically acceptable salt, prodrug, solvate, hydrate, or tautomer thereof), which has a structure:

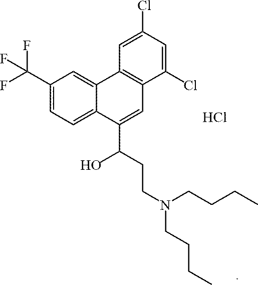

In another independent embodiment, the compound can be N-[3-[[5-cyclopropyl-2-[(2-methyl-3,4-dihydro-1H-isoquinolin-6-yl)amino]pyrimidin-4-yl]amino]propyl]cyclobutanecarboxamide (or a pharmaceutically acceptable salt, prodrug, solvate, hydrate, or tautomer thereof), which has a structure:

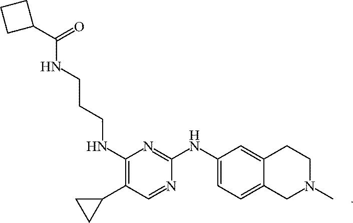

### IV. Method of Making Compound Embodiments

Method embodiments for making the compound embodiments of the present disclosure also are described. Exemplary method embodiments are described in the Examples of the present disclosure.

In some embodiments, a method for making a compound according to Formula I can be made as described below.

In some embodiments for making compounds of Formula I, methyl (1S,2R,3R,4aS,13bR,14aS)-3-hydroxy-2,11-dimethoxy-1,2,3,4,4a,5,7,8,13,13b,14,14a-dodecahydroindolo[2′,3′:3,4]pyrido[1,2-b]isoquinoline-1-carboxylate can be used as a starting material (compound 100 in Scheme 1 below). In particular embodiments, an R⁴ group can be installed on this starting compound using any of the following method embodiments. Any such methods further can be used to provide an R³ group as described above for any of Formulas I, and IA-IE.

In some embodiments, certain R⁴ groups can be installed using Williamson ether synthesis from the reaction of alkyl halide with 100 under basic conditions as illustrated in Scheme 1, thereby providing product 102. Additional embodiments are provided below and representative methods are described in the Examples section.

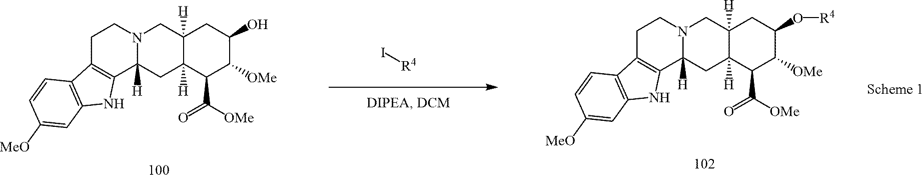

In other embodiments, certain R⁴ groups can be installed by reaction of 100 with a corresponding acyl halide or carboxylic acid as illustrated in Schemes 1A and 1B, respectively, thereby providing product 104.

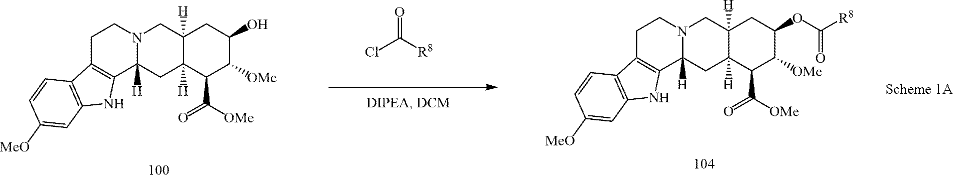

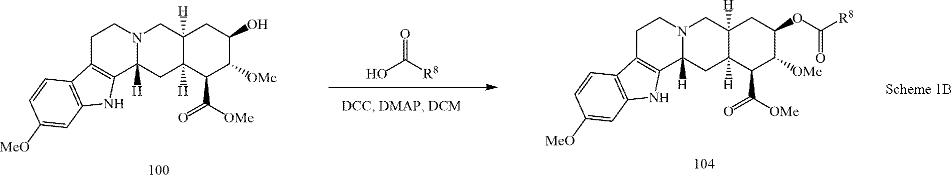

In yet other embodiments, certain R⁴ groups can be installed by reaction of 100 with a corresponding sulfonyl chloride as illustrated in Scheme 1C, to thereby provide product 106.

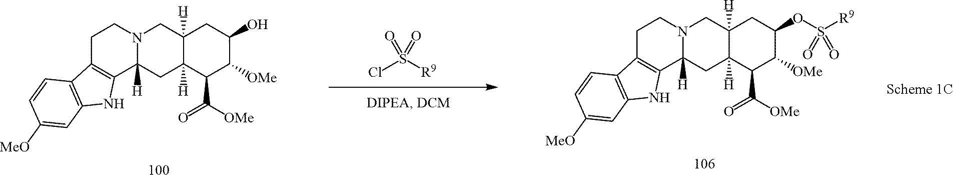

In some embodiments, compounds of Formula I comprising different R² groups (different from the methyl ester groups illustrated in Schemes 1 and 1A-1C) above can be made according to the following method embodiments illustrated in Scheme 1D, providing products 110 or 112.

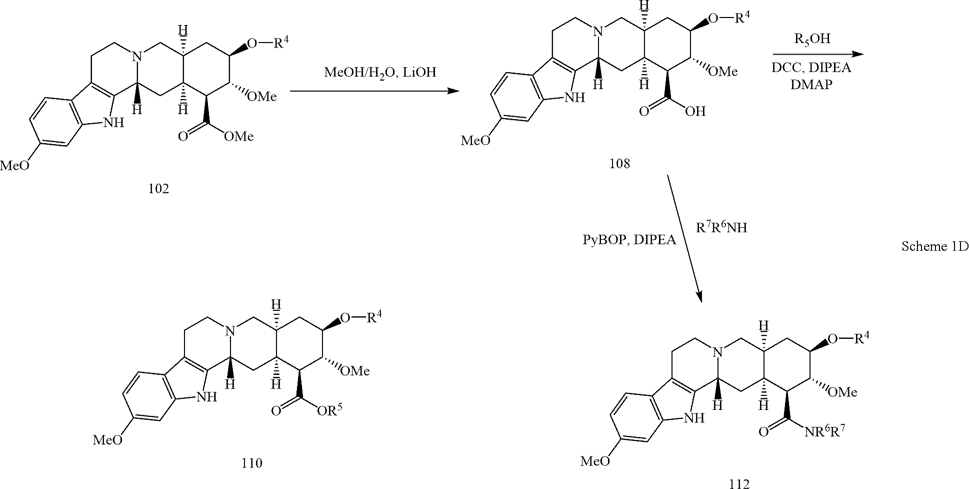

Also disclosed are method embodiments for making compounds represented by Formula II (and/or Formula IIA). In such embodiments, the method can comprise using a starting material 200, illustrated in Scheme 2 below, and coupling it with a corresponding palladium coupling reagent 202, which provides the R^{B} group. While Scheme 2 illustrates a palladium-based coupling reaction using a boronic acid coupling partner (202), other coupling partners can be used, including those suitable for a Stille-based coupling (e.g., R^{B}Sn(Bu)₄, wherein R^{B} can be selected from R^{B} groups described in the definitions section) or a Negishi-based coupling (e.g., R^{B}ZnX′, wherein R^{B} can be selected from R^{B} groups described in the definitions section and X′ can be halogen, triflate, ester, or the like). Additional embodiments are provided below and representative methods are described in the Examples section.

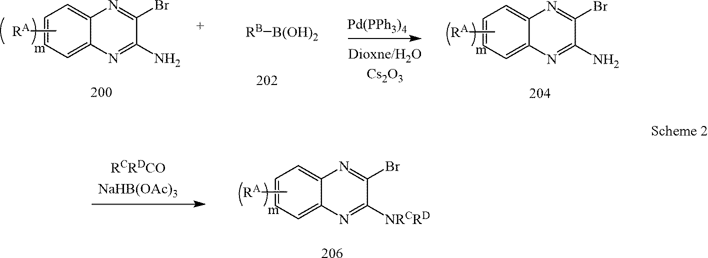

In some embodiments, the method can comprise steps like those outlined in Scheme 2A, wherein X″ can be CH or N (or an oxidized form thereof) and Y can be selected from aromatic (e.g., aryl or heteroaryl); aromatic (e.g., aryl or heteroaryl) comprising one or more substituents selected from halogen, —CN, alkoxy, —OCF₃; or aliphatic (e.g., cyclic aliphatic).

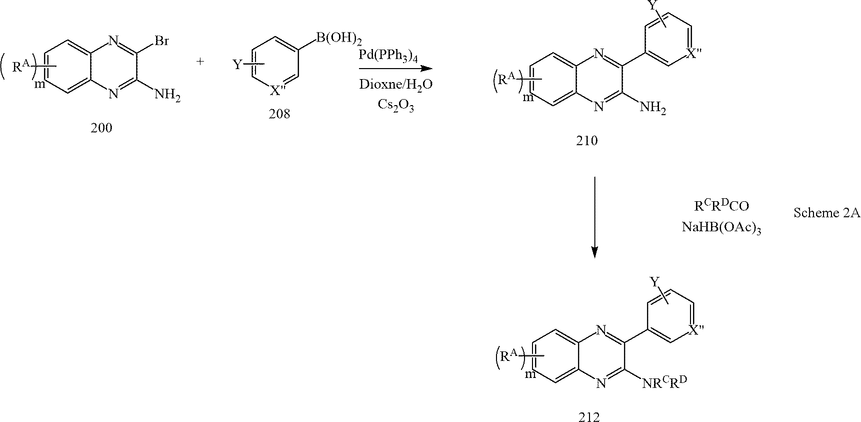

Also disclosed are method embodiments for making compounds represented by Formula III (and/or Formulas IIIA-IIID). In such embodiments, the method can comprise reacting a precursor compound 300 (wherein Z is a halogen, such as chloro) with a suitable coupling component under suitable coupling conditions to provide product 302; or reacting a precursor compound 304 (wherein each of Z′ and Z″ independently can be an amine or a hydroxyl group) with a suitable coupling component under suitable coupling conditions to provide product 306. Additional embodiments are provided below and representative methods are described in the Examples section.

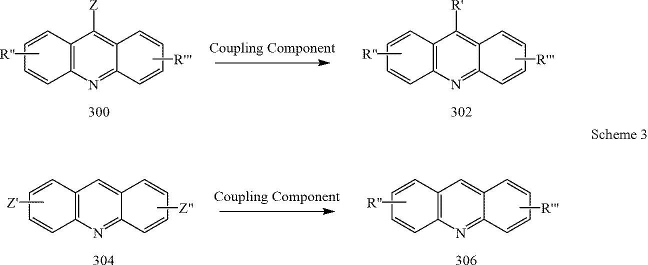

In some embodiments, the method can comprise steps like those outlined in Scheme 3A, wherein the R group of the RONa reagent can be alkyl or heteroalkyl; each Y′ can be selected from amino, heteroaliphatic, amide, or sulfonamide; and m′ is an integer selected from 0 to 5.

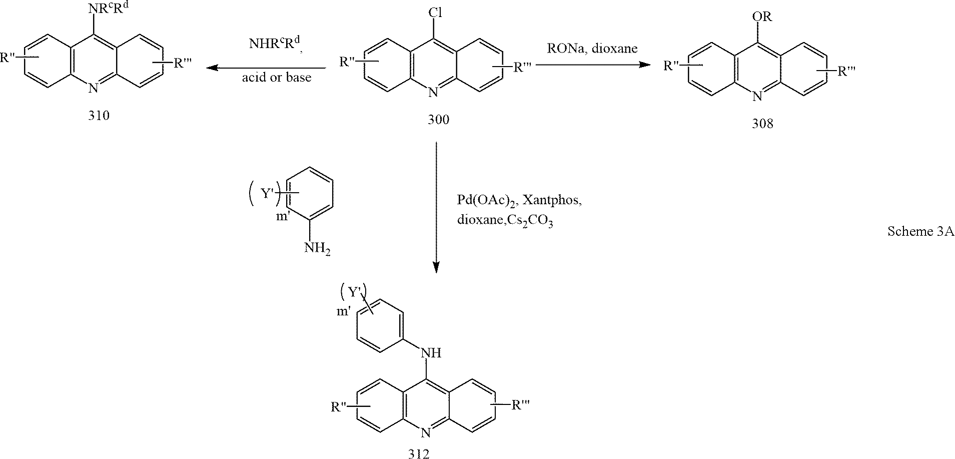

In some embodiments, the method can comprise steps like those outlined in Scheme 3B, which comprise using starting material 314 (wherein the starting material comprises at least one hydrogen atom bound to each of the illustrated amine groups in addition to any R^{a′} or R^{b′} group) to which two of the same or different acyl and/or sulfonyl groups can be coupled using the corresponding acyl or sulfonyl coupling reagent (e.g., an acyl halide and/or a sulfonyl halide). With reference to Scheme 3B, at least one of the R^{a′} or R^{b′} groups attached to each amine of product 316 is an acyl group or a sulfonyl group. Symmetric versions of product 316 can be made, wherein each NR^{a′}R^{b′} group is the same; or asymmetric versions of product 316 can be made, wherein each NR^{a′}R^{b′} group is different.

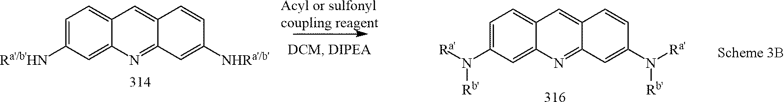

In yet some additional embodiments, the method can comprise steps like those outlined in Scheme 3C, wherein starting material 314 is converted to product 316 using a sequence of protection, addition, and deprotection steps. Such embodiments can be used in certain examples to provide compounds wherein the amine nitrogen is bound to at least one aliphatic, heteroaliphatic, haloaliphatic, or aromatic group.

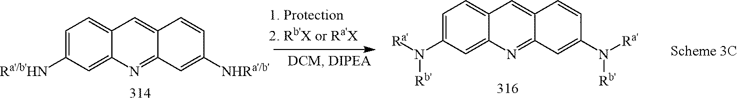

### V. Method of Use

Method embodiments are disclosed herein for treating and/or preventing retinal degeneration in a subject. The method embodiments can include selecting a subject with retinal degeneration, or a subject that is of risk for retinal degeneration. Generally, a therapeutically effective amount of a compound as disclosed herein is administered. In some embodiments, the compound can be 3-(dibutylamino)-1-(1,3-dichloro-6-(trifluoromethyl)phenanthren-9-yl)propan-1-ol hydrochloride, or another pharmaceutically acceptable salt, prodrug, solvate, hydrate, and/or tautomer of 3-(dibutylamino)-1-(1,3-dichloro-6-(trifluoromethyl)phenanthren-9-yl)propan-1-ol. In additional embodiments, the compound can have a structure according one of Formulas I, II, or III, or any of the compounds disclosed herein, including any pharmaceutically acceptable salt, prodrug, solvate, hydrate, or tautomer thereof. This administration is sufficient to treat, inhibit and/or prevent retinal degeneration. In some embodiments, the subject has ongoing photoreceptor degeneration. In further embodiments, the method treats retinal degeneration in the subject.

Various eye conditions may be treated or prevented by using compound embodiments disclosed herein. The conditions include retinal diseases or disorders generally associated with retinal dysfunction or degradation, retinal injury, and/or loss of retinal pigment epithelium. The disclosed methods are of use for treating a retinal degenerative disease, retinal (or retinal pigment) epithelium dysfunction, retinal degradation, retinal (or retinal pigment) epithelial damage. The disclosed methods are also of use for treating loss of retinal pigment epithelium. The methods include administering, such as locally administering, compound embodiments (or a composition thereof) to the eye of the subject.

In some embodiments the retina degenerative disease is Stargardt’s macular dystrophy, retinitis pigmentosa, age related macular degeneration, diabetic retinopathy, Leber congenital amaurosis (LCA), late-onset retinal degeneration, hereditary macular or acquired retinal degeneration, choroideremia, Best disease, Sorsby’s fundus dystrophy, gyrate atrophy, choroideremia, pattern dystrophy, or cone-rod dystrophy. In a specific non-limiting example, the subject has retinitis pigmentosa, LCA, Stargardt’s macular dystrophy, cone-rod dystrophy, choroideremia, or age-related macular degeneration.

In some embodiments, the method can include selecting a subject for treatment. In some embodiments, the method can include selecting a subject with Stargardt’s macular dystrophy, retinitis pigmentosa, age related macular degeneration, diabetic retinopathy, Leber congenital amaurosis, late-onset retinal degeneration, hereditary macular or acquired retinal degeneration, choroideremia, Best disease, Sorsby’s fundus dystrophy, gyrate atrophy, choroideremia, pattern dystrophy, or cone-rod dystrophy. In a specific non-limiting example, the subject has retinitis pigmentosa, LCA, Stargardt’s macular dystrophy, cone-rod dystrophy, choroideremia or age-related macular degeneration. Thus, the method can include selecting a subject with retinal degeneration, such as, but not limited to, a subject with retinitis pigmentosa, LCA, Stargardt’s macular dystrophy, cone-rod dystrophy, choroideremia or age-related macular degeneration. In additional embodiments, subject can have diabetic retinopathy. The methods can include selecting a subject with diabetic retinopathy, or a subject at risk for diabetic retinopathy, such as a diabetic subject. Following selection, the subject is administered an effective amount of one or more compound embodiments as disclosed herein, including any pharmaceutically acceptable salt, prodrug, solvate, hydrate, or tautomer thereof.

In certain embodiments, the presently disclosed methods embodiments can be used to treat any type of retinitis pigmentosa. In some embodiments, the retinitis pigmentosa is caused by mutations in the rhodopsin gene, the peripherin gene, and/or other genes expressed in the rod. The retinitis pigmentosa can be the result of a genetic condition inherited in an autosomal dominant, autosomal recessive or X-linked manner. The X-linked retinitis pigmentosa can be recessive, affecting males, or dominant, so that it affects males and females. The retinitis pigmentosa can be associated with rod-cone retinal degenerations present with central macular pigmentary changes (bull’s eye maculopathy). The retinitis pigmentosa can be choroideremia, which is an X-linked recessive retinal degenerative disease. Generally, the retinitis pigmentosa (RP) is characterized by the progressive loss of photoreceptor cells.

In additional embodiments, the presently disclosed methods can be used to prevent or treat age-related macular degeneration (AMD). In some embodiments, the subject has atrophic AMD (also called “dry” AMD), wherein the subject has symptomatic central vision loss due to retinal atrophy. In other embodiments, the subject has wet AMD.

In further embodiments, the disclosed methods are of use to treat a subject with LCA. In more embodiments the disclosed methods are of use to LCA that has a defect in the CEP290 protein, and thus may have defects in trafficking of ciliary proteins.

In yet other embodiments the subject has Stargardt’s macular dystrophy. In more embodiments, the subject has cone-rod dystrophy. In a further embodiment, the subject has choroideremia.

Diagnosis can utilize tests which examine the fundus of the eye and/or evaluate the visual field. These include electroretinogram, fluorangiography, and visual examination. The fundus of the eye examination aims to evaluate the condition of the retina and to evaluate for the presence of the characteristic pigment spots on the retinal surface. Examination of the visual field makes possible to evaluate the sensitivity of the various parts of the retina to light stimuli. An electroretinogram (ERG) can be used, which records the electrical activity of the retina in response to particular light stimuli and allows distinct valuations of the functionality of the two different types of photoreceptors (e.g., cone cells and rod cells).

Combinations of the compounds can be used, including those combinations that act synergistically. Thus, in any of the disclosed methods, 2, 3, 4 or more compounds can be administered.

In some embodiments, the compound is administered for 10, 15, 20, 25, or 30 days. In further embodiments, the compound is administered for at least 1, 2, 3, 4, 5, 6, 7, 8, 9, 10, 11 or 12 months. In additional embodiments, the compound can be administered for up to six months, or one year, two years, three years, or longer. In some examples, the compound can be administered daily, daily every other day, every three days, or weekly for the specified time period. A sustained release formulation, such as a compound-releasing drug depot or sustained release implant or device, can also be used. In some examples, the compound is administered daily.

Systemic modes of administration include oral and parenteral routes. Parenteral routes include, by way of example, intravenous, intraarterial, intramuscular, intradermal, subcutaneous, intranasal and intraperitoneal routes. Compounds administered systemically may be modified or formulated to target the components to the eye, such as, but not limited to, intra-vitreal administration. In other embodiments, the compound is administered orally.

A suitable oral formulation of a compound is, for example, a tablet or capsule, preferably a tablet containing, for example, about 10, 20, 30 or 40 mg/kg, of the compound. In some embodiments, the compound can be administered at a dose in the range of about 20 mg/kg to about 160 mg/kg per day, such as about 20 mg/kg to about 80 mg/kg, for example, about 20 mg/kg to about 40 mg/kg, either as a single dose or as divided doses. In a specific non-limiting example, this dose is administered daily.

In other embodiments, the compound is administered orally at a dose of about 10 mg/kg to about 80 mg/kg. In other embodiments, a compound is administered orally at a dose of about 40 mg/kg to about 80 mg/kg. In some examples, a compound is administered orally at a dose of about 40, 45, 50, 55, 60, 65, 70, 75 or 80 mg/kg. In specific non-limiting examples, this dose is administered daily.

In further embodiments, for humans, the compound is administered orally at a dose of about 0.8 mg/kg to about 6.5 mg/kg daily (≈10 mg/kg/day, approximately equivalent to a 0.81 mg/kg/day dose in an adult human). In some non-limiting examples, the compound is administered orally at a dose of about 3.2 mg/kg to about 6.5 mg per kg daily. Suitable doses include, but are not limited to, about 0.8 mg/kg, 0.9 mg/kg, 1 mg/kg, 1.1 mg/kg, 1.2 mg/kg, 1.3 mg/kg. 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.;6 mg/kg, 5.7 mg/kg, 5.8 mg/kg, 4.9 mg/kg, 6.0 mg/kg, 6.1 mg/kg, 6.2 mg/kg, 6.3 mg/kg, 6.4 mg/kg and 6.5 mg/kg. The compound can be formulated for administration in any oral formulation, including solid or liquid formulations. The compound can be administered daily.

In one non-limiting example, the compound is administered orally at a dose of about 40 mg/kg to about 80 mg/kg daily for a minimum of 1, 2 3, 4, 5, 6, 7, 8, 9, 10, 11 or 12 months. In other non-limiting examples, the compound is administered orally at a dose of about 0.8 mg/kg to about 6.5 mg/kg daily for a minimum of 1, 2 3, 4, 5, 6, 7, 8, 9, 10, 11 or 12 months. In additional embodiments, the compound can be administered orally at a dose of about 0.8 mg/kg to about 6.5 mg/kg daily for up to six months, or one year, two years, three years, or longer. In some examples, the compound can be administered orally and daily, daily every other day, every three days, or weekly, for the specified time period. In some examples, the compound is administered daily and orally. In some non-limiting examples, the compound is administered orally at a dose of about 40 mg/kg to about 80 mg/kg daily for at least 3 months, 4, 5, 6, 7, 8, 9, 10, 11 or 12 months. In further non-limiting examples, the compound is administered orally at a dose of about 0.8 mg/kg to about 6.5 mg/kg daily for at least 3 months, 4, 5, 6, 7, 8, 9, 10, 11 or 12 months.

The compound can be administered locally to the eye. Local modes of administration include, by way of example, intraocular, intraorbital, subconjunctival, sub-Tenon’s, subretinal or transscleral routes. In an embodiment, significantly smaller amounts of the components (compared with systemic approaches) may exert an effect when administered locally (for example, intravitreally) compared to when administered systemically (for example, intravenously). In one embodiment, the compound is delivered subretinally, e.g., by subretinal injection. Subretinal injections may be made directly into the macular, e.g., submacular injection. Exemplary methods include intraocular injection (e.g., retrobulbar, subretinal, submacular, intravitreal and intrachoridal), iontophoresis, eye drops, and intraocular implantation (e.g., intravitreal, sub-Tenons and sub-conjunctival).

In one embodiment, the system disclosed herein is delivered by intravitreal injection. Intravitreal injection has a relatively low risk of retinal detachment. Methods for administration of agents to the eye are known in the medical arts and can be used to administer components described herein.

Administration may be provided as a single administration, a periodic bolus, or as continuous infusion. In some embodiments, administration is from an internal reservoir (for example, from an implant disposed at an intra- or extra-ocular location - see, for example, U.S. Pat. Nos. 5,443,505 and 5,766,242, the relevant portion of which is incorporated herein by reference) or from an external reservoir (for example, from an intravenous bag). Components can be administered by continuous release for a particular period from a sustained release drug delivery device immobilized to an inner wall of the eye or via targeted transscleral controlled release into the choroid (see, for example, PCT/US00/00207, PCT/US02/14279, Ambati et al., Invest. Opthalmol. Vis. Sci. 41:1181-1185, 2000, and Ambati et al., Invest. Opthalmol. Vis. Sci. 41:1186-1191, 2000, the relevant portion of which is incorporated herein by reference). A variety of devices suitable for administering components locally to the inside of the eye are known in the art and can be selected for use in the present disclosure. See, for example, U.S. Pat. No. 6,251,090, U.S. Pat. No. 6,299,895, U.S. Pat. No. 6,416,777, U.S. Pat. No. 6,413,540, and PCT Application No. PCT/US00/28187, the relevant portions of which are incorporated herein by reference.

Dosage treatment may be a single dose schedule or a multiple dose schedule to ultimately deliver the amount specified above. The doses can be intermittent. Moreover, the subject may be administered as many doses as appropriate. In some embodiments, the subject is administered the compound prior to the onset of a condition.

Individual doses are typically not less than an amount required to produce a measurable effect on the subject and may be determined based on the pharmacokinetics and pharmacology for absorption, distribution, metabolism, and excretion (“ADME”) of the subject composition or its by-products, and thus based on the disposition of the composition within the subject. This includes consideration of the route of administration as well as dosage amount, which can be adjusted for local and systemic (for example, oral) applications. Effective amounts of dose and/or dose regimen can readily be determined empirically from preclinical assays, from safety and escalation and dose range trials, individual clinician-patient relationships, as well as in vitro and in vivo assays. Generally, these assays will evaluate retinal degeneration, or expression of a biological component (cytokine, specific inflammatory cell, microglia, etc.) that affects retinal degeneration. In some embodiments, the dose can be an in vivo dose that corresponds to (i) an in vitro intermittent high dose of 20 µM or 30 µM or (ii) an in vitro continual lose dose of 10 µM as administered in a CEP290-LCA in vitro assay used to determine compound efficacy in improving rhodopsin staining and/or ciliary axoneme growth.

In some embodiments, the subject method results in a therapeutic benefit, such as preventing the development of retinal degeneration, halting the progression of a retinal degeneration, and/or reversing the progression of a retinal degeneration. The subject can have any form of retinal degeneration, as disclosed above.

In some embodiments, the method includes the step of detecting that a therapeutic benefit has been achieved. Measures of therapeutic efficacy will be applicable to the particular disease being modified and a person having at least ordinary skill in the art, with the benefit of the present disclosure, will recognize the appropriate detection methods to use to measure therapeutic efficacy. In further embodiments, the compound embodiments disclosed herein (including any pharmaceutically acceptable salt, prodrug, solvate, hydrate, or tautomer thereof) can increase the number of photoreceptors in the retina, as compared to a control. In yet other embodiments treatment with the compound embodiments disclosed herein (including any pharmaceutically acceptable salt, prodrug, solvate, hydrate, or tautomer thereof) can maintain the thickness of the nuclear layer of photoreceptors in the retina over time. In more embodiments, the compound embodiments disclosed herein (including any pharmaceutically acceptable salt, prodrug, solvate, hydrate, or tautomer thereof) can increase expression of a phototransduction protein, such as an opsin, rhodopsin, and/or rod cyclic GMP phosphodiesterase 6β (PDE6β), as compared to a control. In some embodiments, the compound embodiments disclosed herein (including any pharmaceutically acceptable salt, prodrug, solvate, hydrate, or tautomer thereof) can increase rhodopsin and/or S-opsin expression, as compared to a control. In more embodiments, the compound embodiments disclosed herein (including any pharmaceutically acceptable salt, prodrug, solvate, hydrate, or tautomer thereof) can increase a phototransduction protein, such as (but not limited to) a photoreceptor, as compared to a control. In yet additional embodiments, the compound embodiments disclosed herein (including any pharmaceutically acceptable salt, prodrug, solvate, hydrate, or tautomer thereof) can improve (e.g., increase) ciliary axoneme production and/or elongation, ciliary biogenesis (e.g., ciliary pocket formation), and/or p62 expression. In yet additional embodiments, the compound embodiments disclosed herein (including any pharmaceutically acceptable salt, prodrug, solvate, hydrate, or tautomer thereof) can inhibit fusion of autophagosomes with lysosomes, thereby increasing p62 expression and restoring HDAC6 degradation. Suitable controls include a standard value, the average values in a subject not treated with the compound, or the value in the subject prior to treatment. Suitable exemplary tests are disclosed in the examples.

In some embodiments, therapeutic efficacy can be observed by fundus photography or evaluation of the ERG response. The method can include comparing test results after administration of the subject composition to test results before administration of the subject composition.

As another example, therapeutic efficacy in treating a progressive cone dysfunction may be observed as a reduction in the rate of progression of cone dysfunction, as a cessation in the progression of cone dysfunction, or as an improvement in cone function, effects which may be observed by, such as electroretinography (ERG) and/or cERG; color vision tests; functional adaptive optics; and/or visual acuity tests, for example, by comparing test results after administration of the subject composition to test results before administration of the subject composition and detecting a change in cone viability and/or function. In some embodiments, the compound embodiments disclosed herein (including any pharmaceutically acceptable salt, prodrug, solvate, hydrate, or tautomer thereof) defer photoreceptor loss, reduce photoreceptor function decrement, and/or reduce visual function loss.

In another example, therapeutic efficacy in treating a vision deficiency can be exhibited as an alteration in the individual’s vision, such as in the perception of red wavelengths, green wavelengths, and/or blue wavelengths. Such effects can be observed by using cERG and color vision tests, for example, by comparing test results obtained after administering a compound of the present disclosure to a subject to test results obtained before administering the compound, and detecting a change in cone and rod viability and/or function. In some embodiments, the method includes evaluation morphology and structure preservation and/or ERG.

### VI. Overview of Several Embodiments

Disclosed herein are embodiments of a method of treating retinal degeneration in a subject, comprising administering to the subject a therapeutically effective amount of a compound thereby treating the retinal degeneration in the subject, wherein the compound is selected from a compound having a structure according to a formula selected from Formula I, II, or III

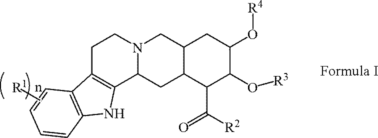

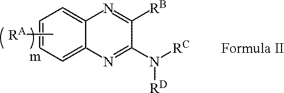

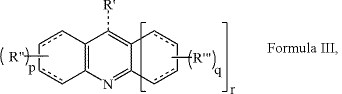

or a pharmaceutically acceptable salt, prodrug, solvate, hydrate, or tautomer thereof; 3-(dibutylamino)-1-(1,3-dichloro-6-(trifluoromethyl)phenanthren-9-yl)propan-1-ol hydrochloride or another pharmaceutically acceptable salt, or a prodrug, solvate, hydrate, or tautomer thereof; wherein,
(i) with reference to Formula I,
   R¹ is heteroaliphatic;
   R² is OR⁵, or NR⁶R⁷, wherein each of R⁵, R⁶, and R⁷ independently is selected from aliphatic, hydrogen, aromatic, or an organic functional group;
   R³ is selected from aliphatic, aromatic, acyl, or sulfonyl;
   R⁴ is selected from acyl, aliphatic, aromatic, or sulfonyl; and
   n is an integer selected from 0 to 4;
(ii) with reference to Formula II,
   R^{A}, is selected from halogen, heteroaliphatic, haloaliphatic, or an organic functional group;
   R^{B} is aromatic; and
   each of R^{C} and R^{D} independently is selected from hydrogen, aliphatic, or heteroaliphatic; and
   m is an integer selected from 0 to 4; and
(iii) with reference to Formula III,
   R′ is selected from aliphatic, aromatic, halogen, heteroaliphatic, haloaliphatic, or an organic functional group;
   each R″ independently is selected from halogen, heteroaliphatic, or amino;
   each R‴ independently is selected from halogen, heteroaliphatic, or amino;
   p is an integer selected from 0 to 4;
   q is an integer selected from 0 to 4; and
   r is an integer selected from 0 or 1.

In some embodiments, the subject has retinitis pigmentosa, LCA, Stargardt’s macular dystrophy, cone-rod dystrophy, choroideremia or age-related macular degeneration.

In any or all of the above embodiments, the compound is administered orally.

In any or all of the above embodiments, the compound is administered locally to the eye of the subject.

In any or all of the above embodiments, the compound is administered intravitreally.

In any or all of the above embodiments, the subject is human.

In any or all of the above embodiments, the compound maintains thickness of a nuclear layer of photoreceptors in a retina of the eye of the subject.

In any or all of the above embodiments, the compound increases expression of a photoreceptor ciliary opsin and/or a phototransduction protein in the eye of the subject.

In any or all of the above embodiments, the photoreceptor ciliary opsin is rhodopsin or S-opsin, or rod cyclic GMP phosphodiesterase 6β (PDE6β).

In any or all of the above embodiments, the compound increases the number of photoreceptor cells in the subject.

In any or all of the above embodiments, the method further comprises evaluating the vision of the subject.

In any or all of the above embodiments, the method comprises performing electroretinography on the subject.

In any or all of the above embodiments, the compound has a structure according to any one of Formulas IA, IC, or IE, or a pharmaceutically acceptable salt, prodrug, solvate, hydrate, or tautomer thereof

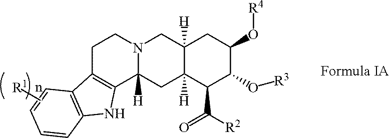

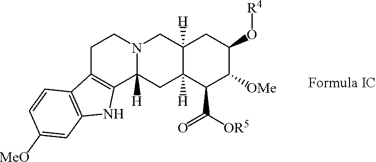

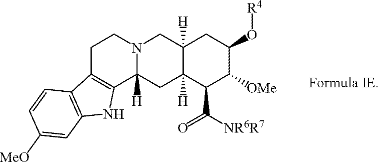

In any or all of the above embodiments, the compound has a structure according to Formula I, IA, IC, or IE, or a pharmaceutically acceptable salt, prodrug, solvate, hydrate, or tautomer thereof, wherein:
R¹is alkoxy;
R² is -OR⁵, or -NR⁶R⁷, wherein each of R⁵, R⁶, and R⁷ independently is selected from hydrogen, alkyl, heteroaryl, or aryl;
R³ is selected from alkyl, heteroaryl, aryl, sulfonyl, or acyl;
R⁴ is selected from acyl, alkyl, heteroaryl, aryl, or sulfonyl;
n is 0, 1, 2, 3, or 4.

In any or all of the above embodiments, the compound has a structure according to Formula I, IA, IC, or IE, or a pharmaceutically acceptable salt, prodrug, solvate, hydrate, or tautomer thereof, and wherein R⁴ is selected from

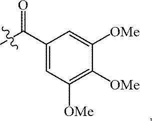

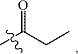

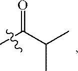

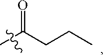

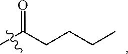

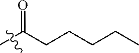

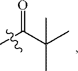

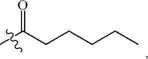

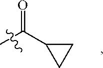

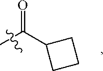

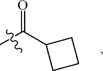

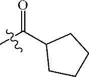

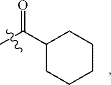

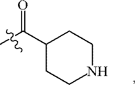

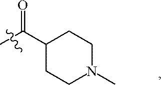

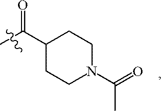

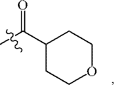

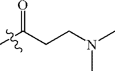

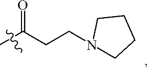

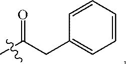

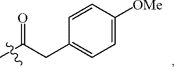

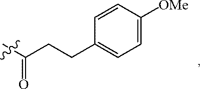

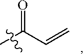

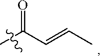

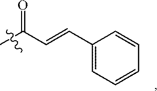

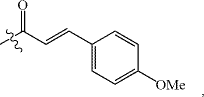

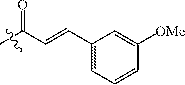

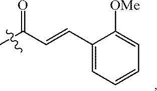

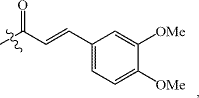

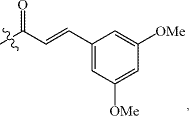

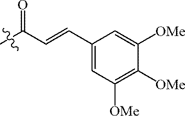

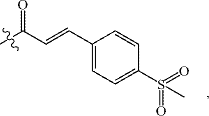

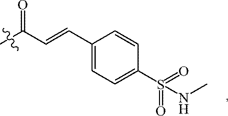

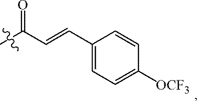

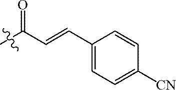

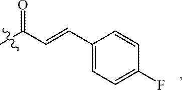

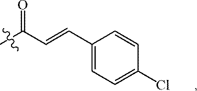

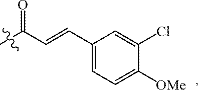

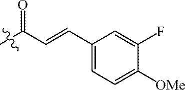

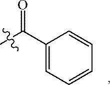

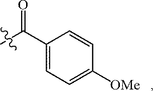

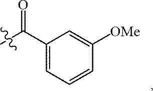

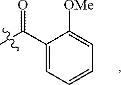

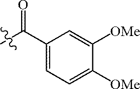

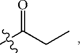

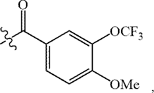

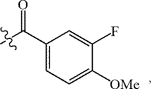

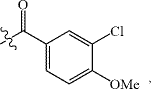

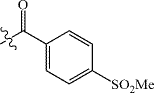

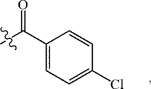

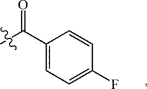

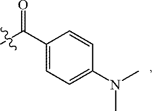

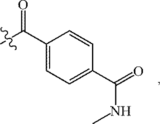

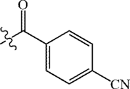

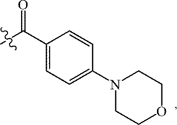

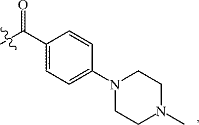

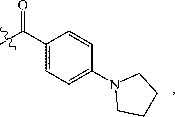

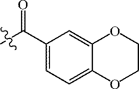

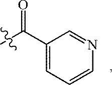

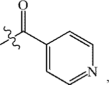

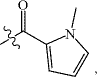

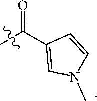

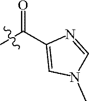

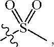

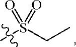

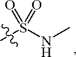

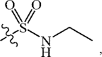

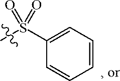

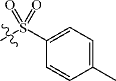

In any or all of the above embodiments, the compound is selected from

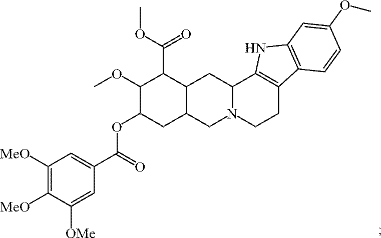

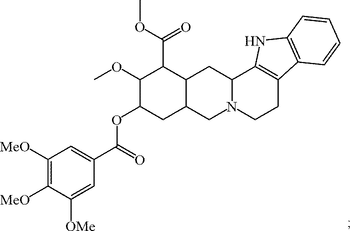

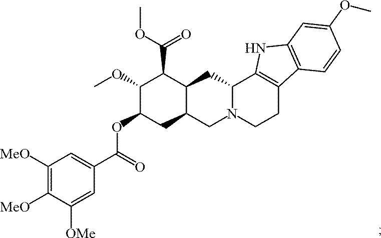

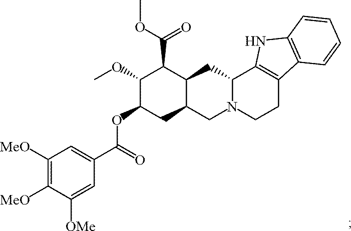

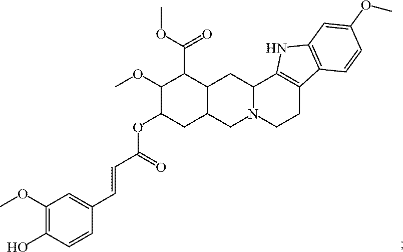

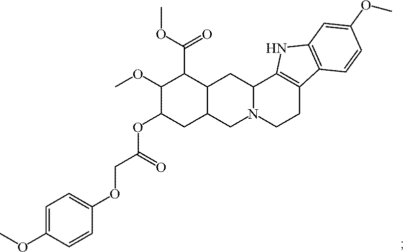

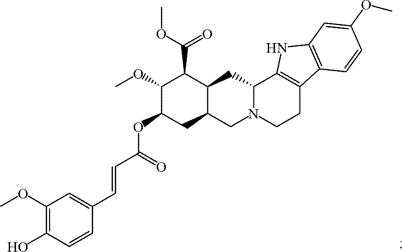

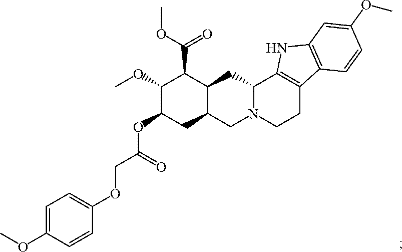

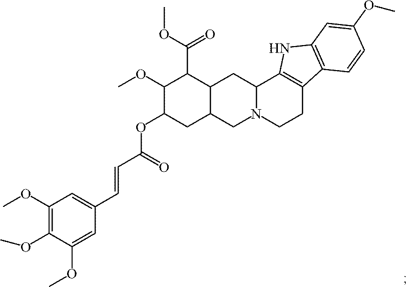

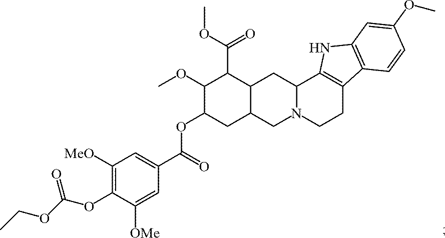

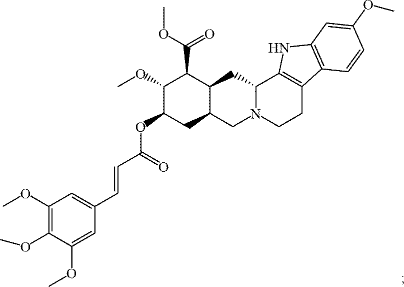

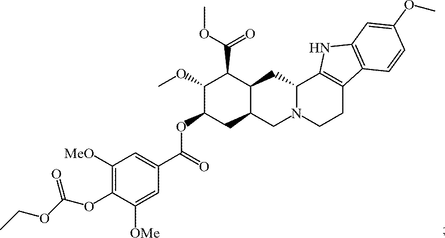

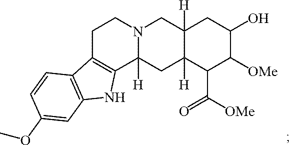

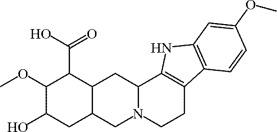

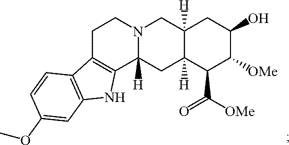

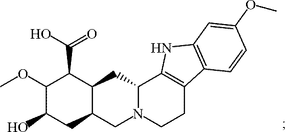

or a pharmaceutically acceptable salt, prodrug, solvate, hydrate, or tautomer thereof.

In any or all of the above embodiments, the compound has a structure according to Formula IIA, or a pharmaceutically acceptable salt, prodrug, solvate, hydrate, or tautomer thereof

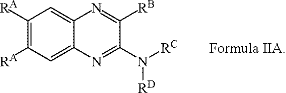

In any or all of the above embodiments, the compound has a structure according to Formula II or IIA, or a pharmaceutically acceptable salt, prodrug, solvate, hydrate, or tautomer thereof, wherein:
each R^{A} independently is selected from halogen, —OMe, —CN, or —CF₃;
R^{B} is selected from aryl; aryl comprising one or more substituents selected from halogen, —CF₃, —CN, —OH, alkyl, or alkoxy; heteroaryl; heteroaryl comprising one or more substituents selected from halogen, —CF₃, —CN, —OH, alkyl, or alkoxy;
each of R^{C} and R^{D} independently is selected from hydrogen, alkyl, or amino; and
m is 0, 1, 2, 3, or 4.

In any or all of the above embodiments, the compound has a structure according to Formula II or IIA, or a pharmaceutically acceptable salt, prodrug, solvate, hydrate, or tautomer thereof, and R^{B} is selected from

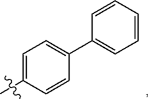

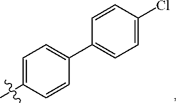

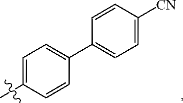

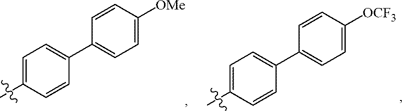

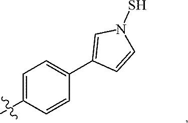

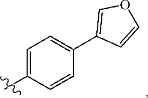

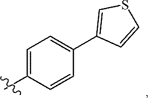

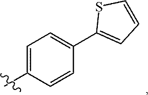

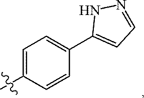

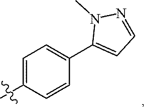

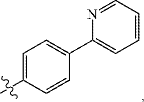

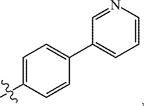

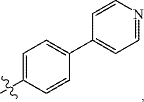

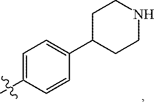

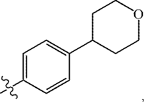

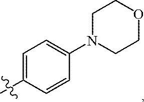

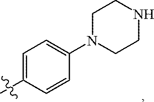

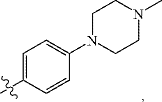

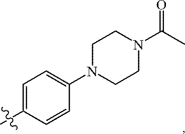

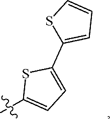

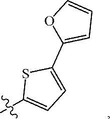

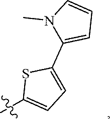

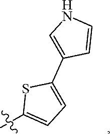

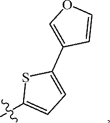

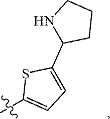

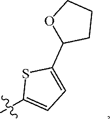

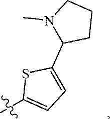

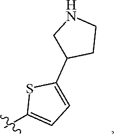

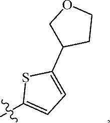

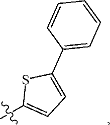

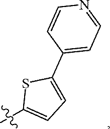

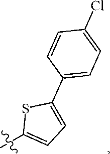

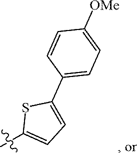

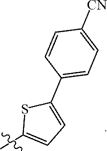

In any or all of the above embodiments, the compound is

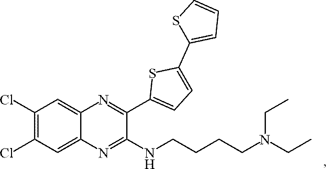

or a pharmaceutically acceptable salt, prodrug, solvate, hydrate, or tautomer thereof

In any or all of the above embodiments, the compound has a structure according to Formulas IIIA-IIID, or a pharmaceutically acceptable salt, prodrug, solvate, hydrate, or tautomer thereof

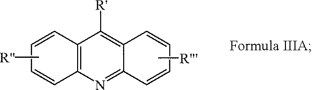

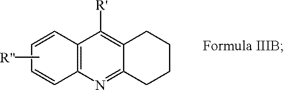

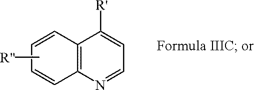

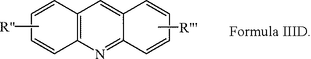

In any or all of the above embodiments, the compound has a structure according to Formula III or IIIA-IIID, or a pharmaceutically acceptable salt, prodrug, solvate, hydrate, or tautomer thereof, and wherein
R′ is selected from halo, —CN; —CF₃; —OCF₃; alkyl; heteroalkyl comprising one or more nitrogen atoms, one or more oxygen atoms, one or more sulfur atoms, or a combination thereof; or aminoaryl;
R″ is selected from halogen, alkoxy, or -NR^{a′}R^{b′}, wherein each of R^{a′} and R^{b′} independently is selected from alkyl, heteroalkyl, benzyl, acyl, sulfonyl;
R‴ is selected from halogen, alkoxy, or -NR^{a′}R^{b′}, wherein each of R^{a′} and R^{b′} independently is selected from alkyl, heteroalkyl, benzyl, acyl, or sulfonyl;
p and q independently is an integer selected from 0, 1, 2, 3, or 4; and
r is 0 or 1.

In any or all of the above embodiments, the compound has a structure according to Formula III or IIIA-IIID, or a pharmaceutically acceptable salt, prodrug, solvate, hydrate, or tautomer thereof, wherein R′ is selected from

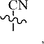

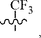

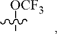

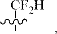

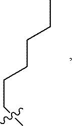

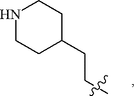

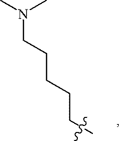

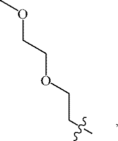

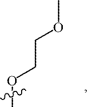

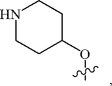

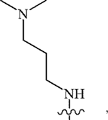

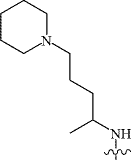

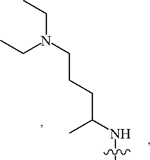

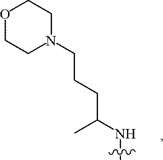

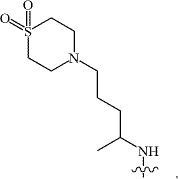

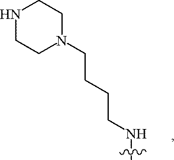

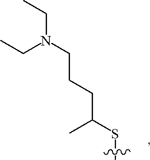

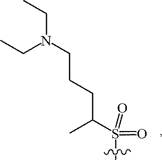

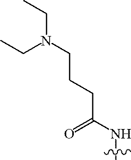

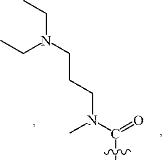

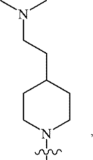

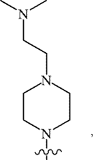

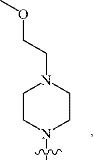

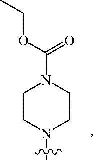

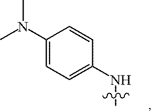

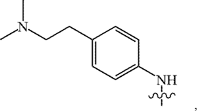

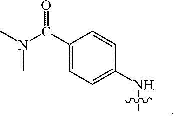

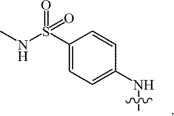

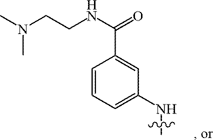

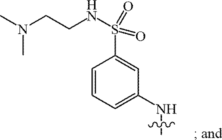

wherein R″ and R‴ independently is -NR^{a′}R^{b′}, wherein one of R^{a′} and R^{b′} is H and the other is selected from

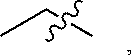

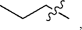

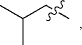

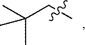

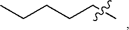

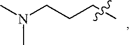

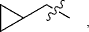

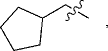

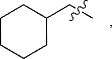

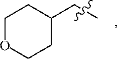

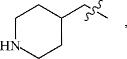

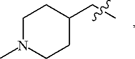

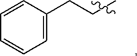

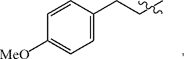

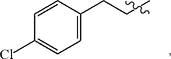

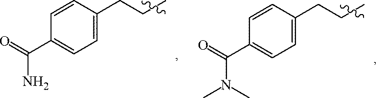

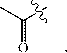

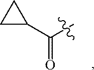

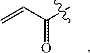

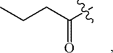

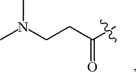

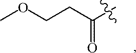

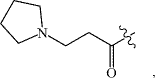

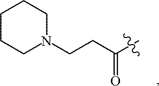

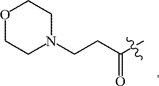

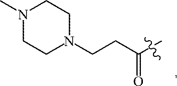

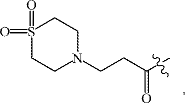

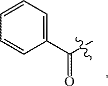

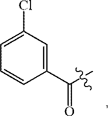

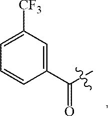

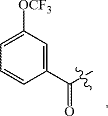

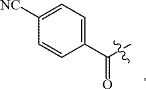

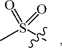

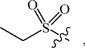

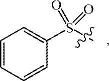

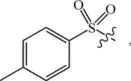

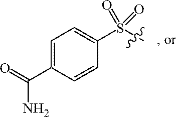

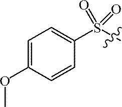

In any or all of the above embodiments, the compound is selected from

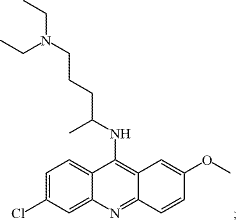

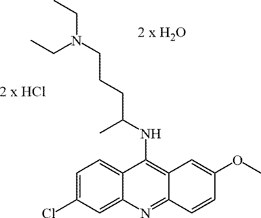

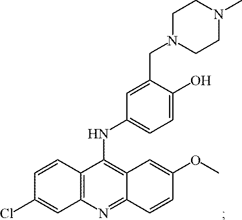

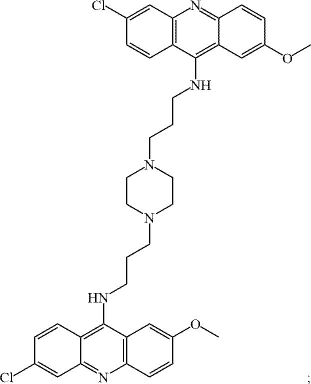

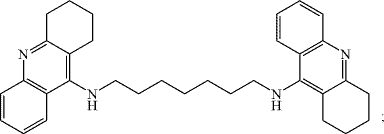

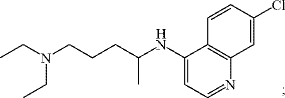

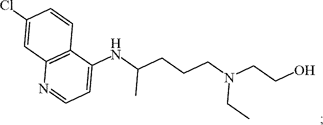

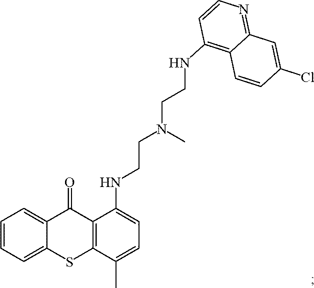

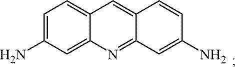

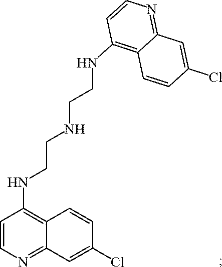

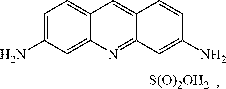

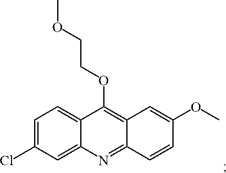

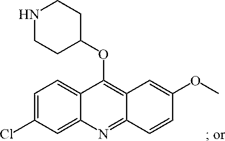

a pharmaceutically acceptable salt, prodrug, solvate, hydrate, or tautomer thereof.

In any or all of the above embodiments, the compound is selected from

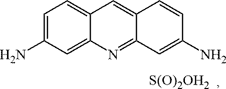

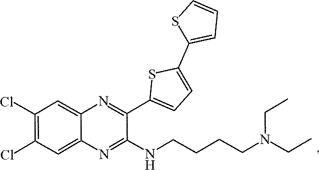

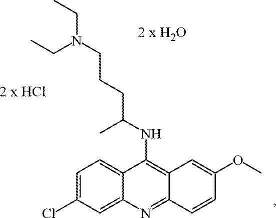

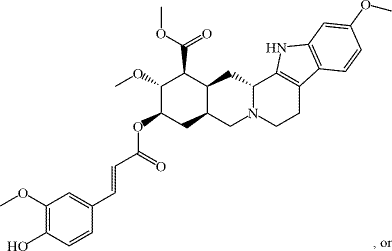

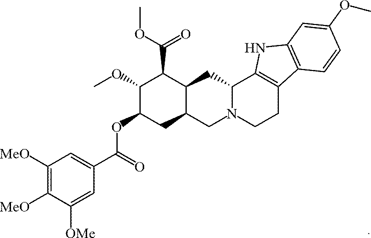

Also disclosed herein are embodiments of a composition, comprising a therapeutically effective amount of a compound according to any or all of the above embodiments for use in treating retinal degeneration in a subject.

In any or all of the above embodiments, the composition is formulated for oral administration.

In any or all of the above embodiments, the composition is included in a dosage form.

In any or all of the above embodiments, the composition is formulated for local administration to the eye.

In any or all of the above embodiments, the composition is formulated for intravitreal administration.

In any or all of the above embodiments, the composition further comprises a therapeutically acceptable excipient.

Also disclosed herein are embodiments of a composition comprising a therapeutically effective amount of a compound for use in the method of any one of any or all of the above embodiments, wherein the compound is selected from 3-(dibutylamino)-1-(1,3-dichloro-6-(trifluoromethyl)phenanthren-9-yl)propan-1-ol hydrochloride or another pharmaceutically acceptable salt, or a prodrug, solvate, hydrate, or tautomer thereof; or a compound having a structure according to a formula selected from Formula I, II, or III

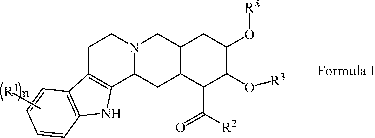

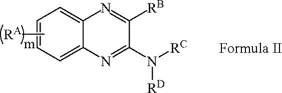

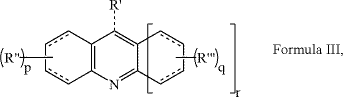

or a pharmaceutically acceptable salt, prodrug, solvate, hydrate, or tautomer thereof; wherein,
with reference to Formula I,
   R¹ is heteroaliphatic;
   R² is OR⁵, or NR⁶R⁷, wherein each of R⁵, R⁶, and R⁷ independently is selected from aliphatic, hydrogen, aromatic, or an organic functional group;
   R³ is selected from aliphatic, aromatic, acyl, or sulfonyl;
   R⁴ is selected from acyl, aliphatic, aromatic, or sulfonyl; and
   n is an integer selected from 0 to 4;
with reference to Formula II,
   R^{A}, is selected from halogen, heteroaliphatic, haloaliphatic, or an organic functional group;
   R^{B} is aromatic; and
   each of R^{C} and R^{D} independently is selected from hydrogen, aliphatic, or heteroaliphatic; and
   m is an integer selected from 0 to 4; and
with reference to Formula III
   R′ is selected from aliphatic, aromatic, halogen, heteroaliphatic, haloaliphatic, or an organic functional group;
   each R″ independently is selected from halogen, heteroaliphatic, or amino;
   each R‴ independently is selected from halogen, heteroaliphatic, or amino;
   p is an integer selected from 0 to 4;
   q is an integer selected from 0 to 4; and
   r is an integer selected from 0 or 1.

### VII. EXAMPLES

The disclosure is illustrated by the following non-limiting Examples.

### Example 1

The ether analogs are prepared through Williamson ether synthesis from the reaction of an alkyl halide with 100 under basic conditions (1). The acyl-functionalized analogs are synthesized from the reaction of 100 with carboxylic acids under the coupling reagents (3) or corresponding acyl chloride (2). Sulfonyl analogs are produced from the reaction of 100 with corresponding sulfonyl chloride (4).

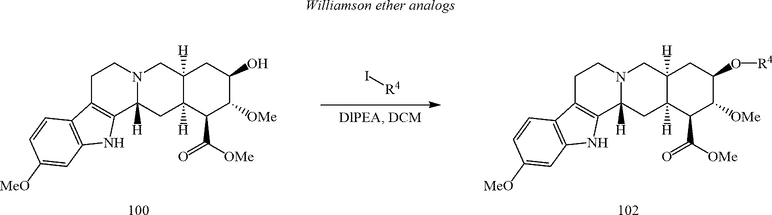

In specific examples, reserpic acid methyl ester 100 is dissolved in toluene, then R⁴I and Ag₂O are added and the resulting mixture is heated in an 80° C. oil bath for 6-12 hours, after which most of the starting material 100 is converted. The product is then filtered and the filtrate is collected, concentrated, and purified using a CombiFlash® purification system.

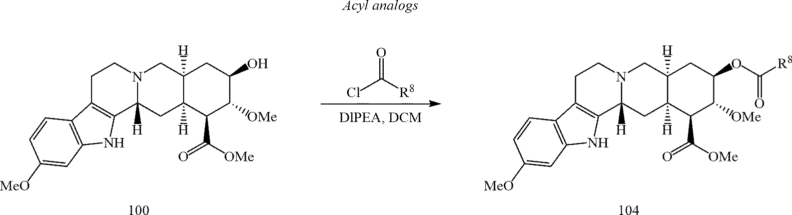

Reserpic acid methyl ester 100 and DIPEA are dissolved in DCM and the resulting mixture is cooled in a 4° C. in ice-water bath. The acyl halide reagent (obtained either from a commercial source or made according to procedures known in the art), are added (as a solution in DCM) dropwise over 5 minutes. The reaction mixture is then allowed to warm to room temperature over a period of 1-5 hours. Once the reaction is finished (as monitored by LCMS to confirm that all starting material is converted), it is filtered to remove insoluble salts and the filtrate is collected, concentrated, and purified using a CombiFlash® purification system.

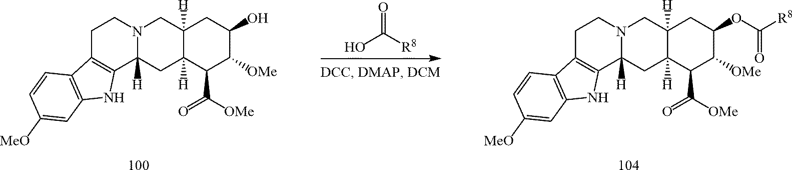

To a solution of a carboxylic acid, coupling reagent (DCC, EDC), DIPEA and DPAM in DCM, which are cooled into 4° C. in ice-water bath, is added reserpic acid methyl ester 100. Then, the reaction mixture is allowed to warm to room temperature over a period of 12-24 hours. Once the reaction is finished (as monitored by confirm that all starting material is converted), it is filtered to remove insoluble salts and the filtrate is collected, concentrated, and purified using a CombiFlash® purification system.

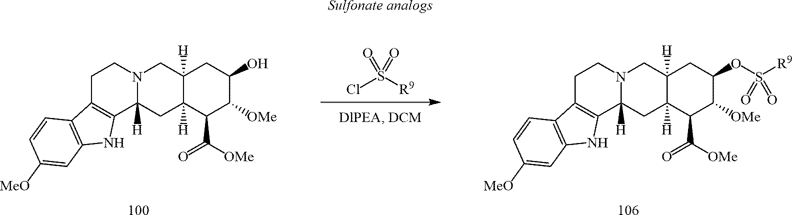

Reserpic acid methyl ester 100 and DIPEA are dissolved in DCM, and the resulting mixture is cooled into 4° C. in an ice-water bath. A sulfonyl chloride reagent (obtained either from a commercial source or made according to procedures known in the art), in a DCM solution, is added dropwise over 5 minutes. The reaction mixture is allowed to warm to room temperature over a period of 1-5 hours. Once the reaction is finished (as monitored by confirm that all starting material is converted), it is filtered to remove insoluble salts and the filtrate is collected, concentrated, and purified using a CombiFlash® purification system.

### Example 2

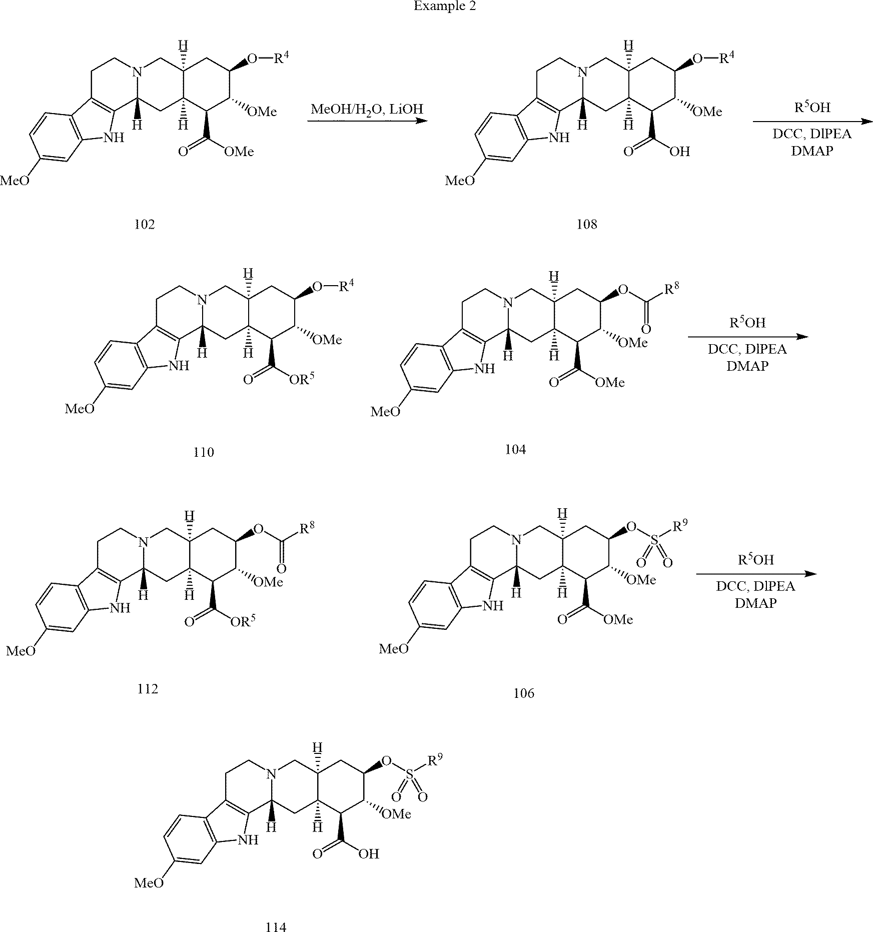

To a solution of a precursor (102, 104, or 106) dissolved with MeOH/H₂O is added LiOH. The resulting mixture is stirred under room temperature for 20-48 hours until most of starting material is converted. The mixture is concentrated, dissolved with DCM/Water, acidified by HCl, then collected the organic phase, which is dried under Na₂SO₄, filtrated, concentrated, dried under high vacuum, ready for next-step to use without further purification. While the above scheme shows the method for preparing the corresponding ester products (110, 112, and 114), amides also can be prepared by replacing the alcohol reagent (R⁵OH) with an amine reagent (e.g., R⁶R⁷NH) and DIPEA and benzotriazol-1-yl-oxytripyrrolidinophosphonium hexafluorophosphate (or “PyBOP”).

### Example 3

In this example, compounds according to Formula II are prepared. In some examples, a precursor 200 is used in a Suzuki coupling with boronic acid 208, which are commercially available or can be prepared using methods known to those of ordinary skill in the art with the benefit of the present disclosure. Cs₂CO₃ and the boronic acid 208 are added to precursor 200 in dioxane/H₂O=10:1, which is degassed by nitrogen gas bubbling, and to which is added Pd(PPh₃)₄. The reaction mixture is capped and irradiated through microwave at 80° C. for 1 hour. The resulting solution is filtered, concentrated, and purified using CombiFlash® purification system to provide product 210. To a solution of 210 in DCE is added the aldehyde reagent and NaBH(OAc)₃. The resulting mixture is stirred for 12-24 hours and monitored by LCMS. The mixture is concentrated and purified using a CombiFlash® purification system to provide product 212.

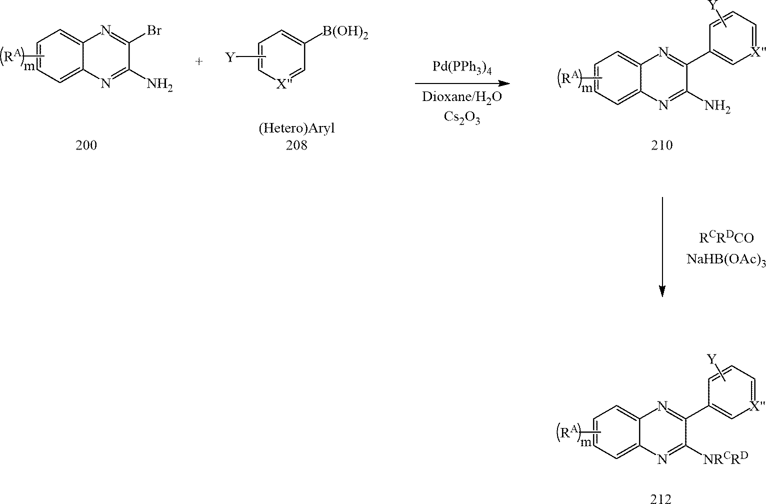

### Example 4

In this example, compounds of Formula III are made. In some examples, precursor 318 can be converted to product 320, wherein R is as recited herein for Scheme 3A. The “RONa” reagent can be purchased commercially or prepared by reacting the corresponding alcohol with Na or NaOH. Then to a solution of precursor 318 in dioxane is added RONa, and the reaction vessel is capped and the reaction mixture was heated at 100-120° C. for 10-24 hours. The reaction mixture is concentrated and then purified using a CombiFlash® purification system to provide product 320.

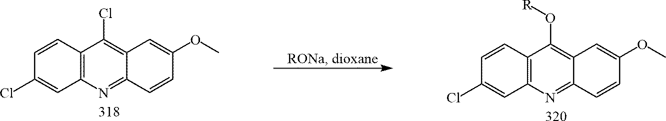

In some additional examples, compounds like amine-functionalized Scheme compound 310 (Scheme 3A) can be made using methods as illustrated in Scheme 3A. In some examples, to a solution of starting material 318 in DMF is added K₂CO₃, and the desired amine reagent. The reaction vessel is capped and the reaction mixture is heated at 100-120° C. for 10-24 hours. The reaction mixture is concentrated and then purified using a CombiFlash® purification system to provide a product according to Formula 310 of Scheme 3A.

In some other examples, to a solution of starting material 318 in EtOH is added HCl dissolved in dioxane and the desired amine reagent. The reaction vessel is capped and the reaction mixture is heated at 100-120° C. for 10-24 hours. The reaction mixture is concentrated and then purified using a CombiFlash® purification system to provide a product according to Formula 310 of Scheme 3A.

In yet additional examples, to a solution of starting material 318 is added Cs₂CO₃ and an aryl amine coupling partner in dioxane. The reaction vessel is degassed by nitrogen gas bubbling and then Pd(OAc)₂ and Xantphos are added. Then reaction vessel is capped and irradiated through microwave at 80° C. for 1 hour. The reaction mixture is filtered, concentrated, and then purified using a CombiFlash® purification system to provide a product according to Formula 312 of Scheme 3A.

### Example 5

In this example, compounds according to Scheme 3B are made. In particular embodiments, compounds having a formula 316 (Scheme 3B) are made by providing a solution of acridine-3,6-diamine and DIPEA dissolved in DCM and cooling it in a 4° C. in ice-water bath. Then, the desired acyl chloride or sulfonyl chloride is added dropwise over 5 minutes. The reaction is allowed to warm to room temperature, and is further stirred for 1-3 hours. The reaction mixture is filtered to remove the insoluble salt, the filtrate is collected and concentrated and then purified using a CombiFlash® purification system to product a compound according to formula 316 from Scheme 3B. In these embodiments, compound 316 is a symmetrical amine.

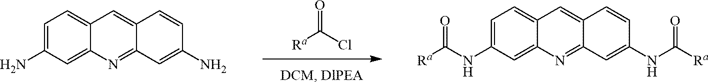

Asymmetric amines can be made by using different coupling partners as illustrated in the scheme below:

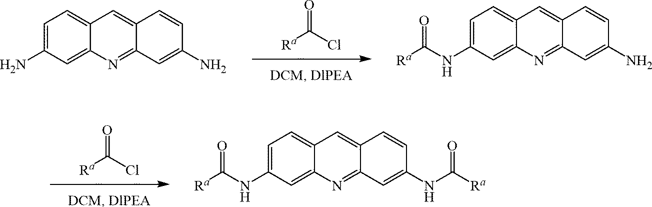

Sulfonyl-containing compounds are made as illustrated below. In particular embodiments, a solution of acridine-3,6-diamine and DIPEA dissolved in DCM is cooled in a 4° C. in ice-water bath, and sulfonic acid chloride is added dropwise over 5 minutes. The reaction is allowed to warm to room temperature, and is further stirred for 1-3 hours. The reaction mixture is filtered to remove the insoluble salt, the filtrate is collected and concentrated and then purified using a CombiFlash® purification system to provide the sulfonyl-containing product.

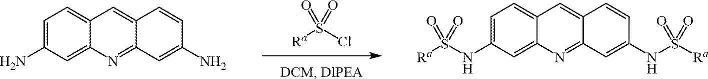

In yet additional examples, other amine compounds can be made by mixing a solution of acridine-3,6-diamine and NaHCO₃ dissolved in THF/water with Boc₂O. The resulting mixture is stirred overnight and extraction is conducted to provide a crude Boc-protected acridine-3,6-diamine. Deprotonation is performed using NaH and the desired R^{a′} or R^{b′} group is added. The Boc protecting group is then removed using TFA.

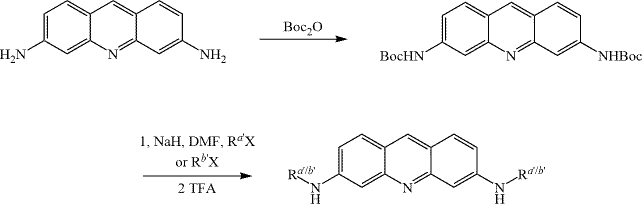

### Example 6

Neural retina in organoids derived from induced pluripotent stem cells (iPSCs) of CEP290-LCA subjects display disease-associated defects - Leber congenital amaurosis (LCA) is an early onset inherited blinding disease that is caused by defects in over 20 different genes. In addition to photoreceptor development and/or function, genetic defects associated with LCA can impact other tissues and present a syndromic clinical phenotype. CEP290 is a cilia-centrosomal protein that is a critical component of transition zone and likely controls trafficking of ciliary proteins. Defects in CEP290 can result in multiple syndromic phenotypes with LCA believed to be towards the milder spectrum.

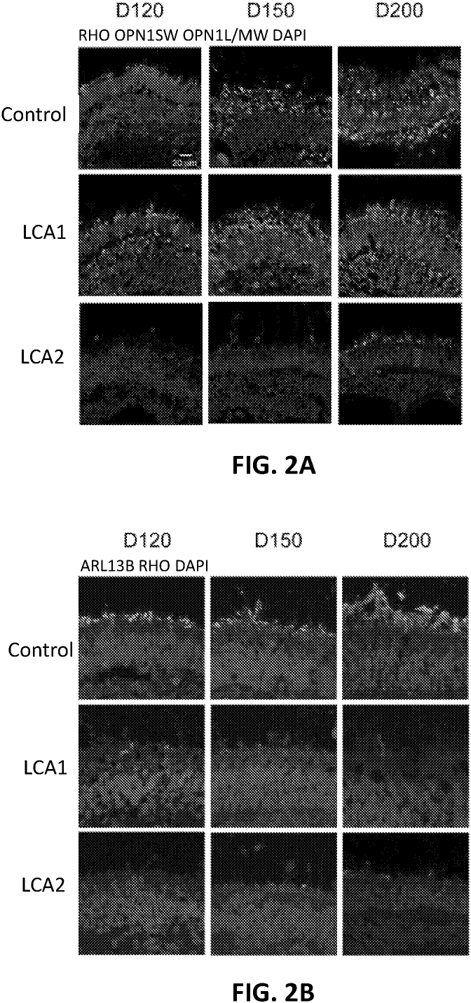

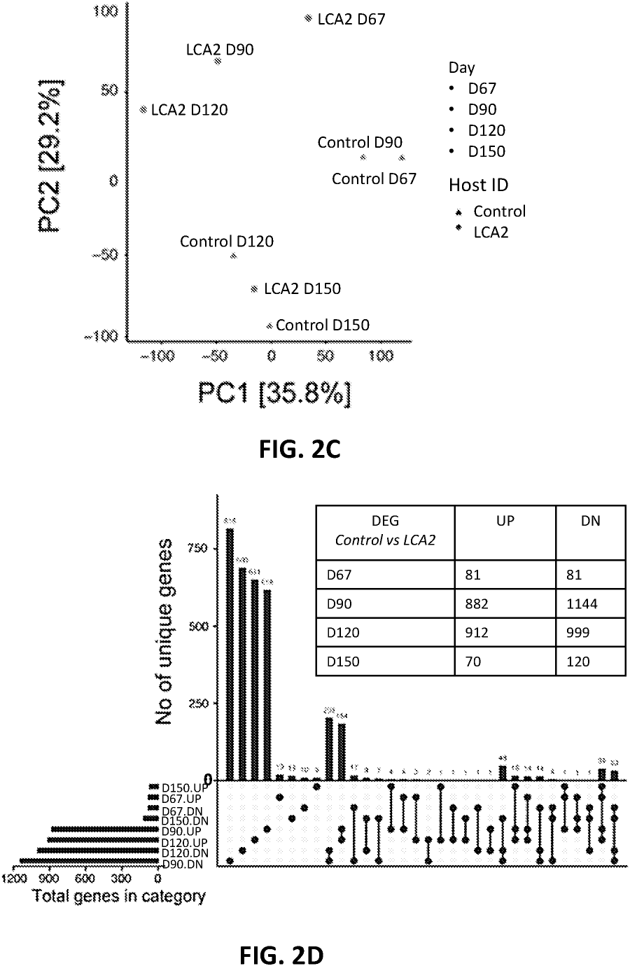

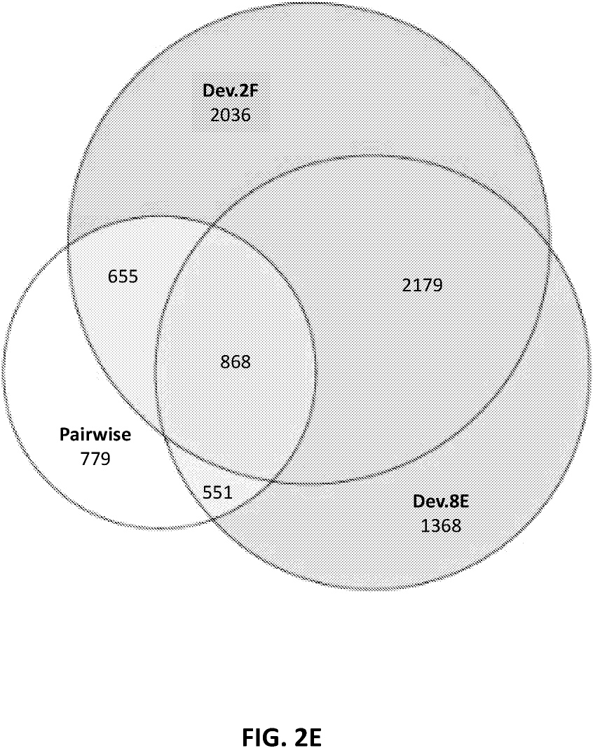

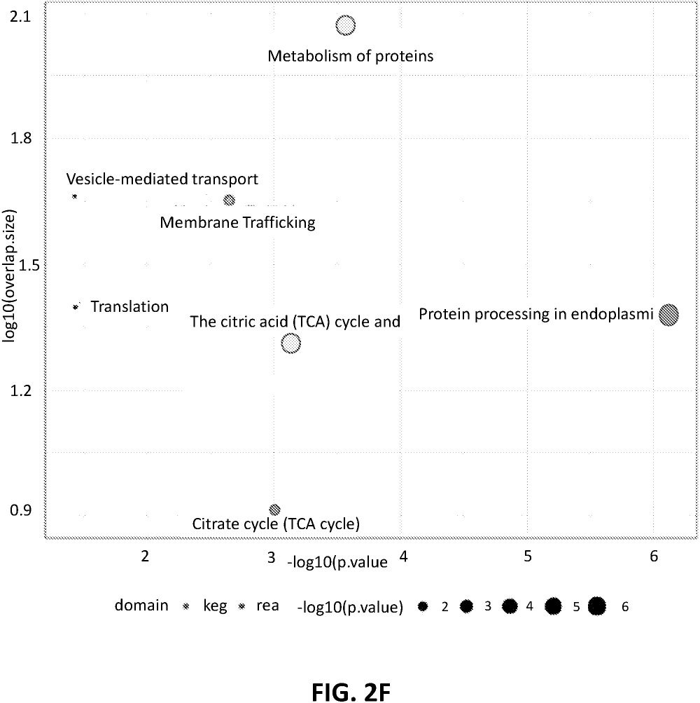

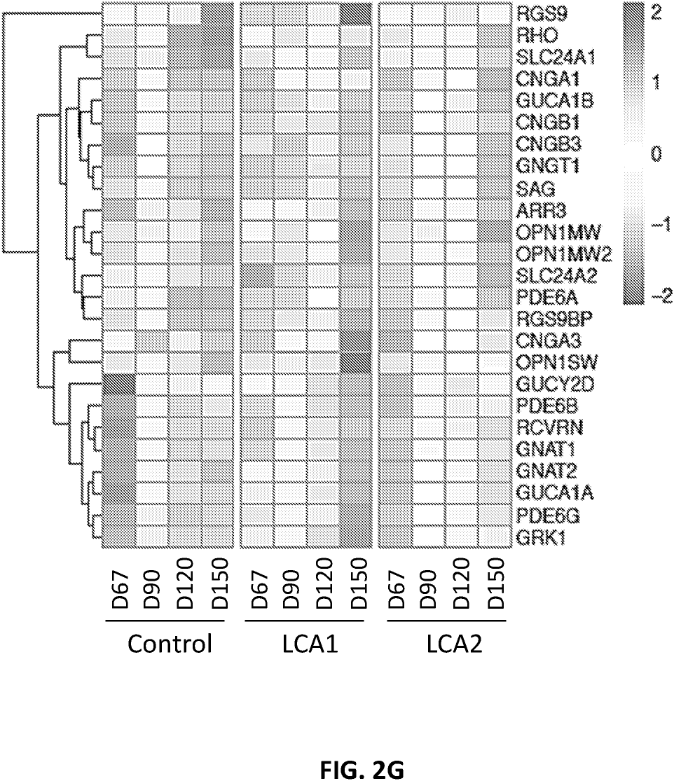

Human pluripotent stem cell (PSC), including embryonic stem cells (ESC) and iPSCs, can be differentiated into retinal organoids with laminated neural retina and photoreceptors with rudimentary outer segment-like structure. To investigate whether the human organoid culture system can recapitulate disease-associated phenotypes observed in CEP290-LCA subjects, a family comprised of a phenotypically normal mother (control) and her two LCA offspring (LCA1 and LCA2) was recruited. Control and subject iPSCs were reprogrammed from fibroblasts and differentiated into retinal organoids. Aberrant phenotypes were identified in subject retinal organoids in comparison to the control. In control organoids, the rod photoreceptor opsin - rhodopsin - was evident at differentiation day (D) 120, followed by its polarization to the apical side of neural retina at D150, and transport to the outer segment region by D200 (FIG. 2A). However, in LCA1 organoids, although rhodopsin could be observed throughout development, it could not be delivered to the outer segments and remained mis-localized in the cell body. LCA2 organoids displayed even more severe phenotypes, as shown by the lack of robust rhodopsin expression. Although cone opsin OPN1SW and OPN1MW was less robust compared to the control neural retina, no significant morphological difference could be observed in cone photoreceptors between control and subject organoids. Immunostaining of connecting cilia and ciliary axoneme marker ARL13B revealed that aberrant photoreceptor development in subject organoids could be caused by ciliary defects in photoreceptors (FIG. 2B). ARL13B staining was concentrated in the connecting cilia of control photoreceptors and elongated along the differentiation process as the outer segment developed. In contrast, consistently in both subject organoids, the photoreceptors demonstrated aberrant development of the connecting cilia and lacked outer segment biogenesis.

To determine gene/signaling pathway signatures in CEP290-LCA subject organoids for understanding disease mechanisms and evaluating effective treatments, control and subject organoid samples were harvested at D67, D90, D120 and D150, and a transcriptome analysis was performed. Principle component analysis showed that control and subject organoid samples roughly separated into two groups across differentiation, suggesting discrepancies in gene profiles between control and subject samples (FIG. 2C). Differential expression analysis revealed the largest discrepancies between control and subject samples occurred at D90 and D120, with 2026 and 1911 differentially expressed (DE) genes, respectively, compared to 162 at D67 and 190 at D150 (FIG. 2D). To isolate the DE genes caused by mutations but not development, an age-matched pairwise comparison was performed between control and subject transcriptomes and the DE genes that were due to developmental stage were removed (FIG. 2E). The 779 unique genes in this analysis belonged to signaling pathways associated with metabolism of proteins, vesicle-mediated transport, membrane trafficking, translation, the citric acid cycle and protein processing in endoplasmic reticulum (FIG. 2F). Notably, expression of phototransduction genes, which are important for photoreceptor function, were mostly down-regulated in subject organoids (FIG. 2G).

### Example 7

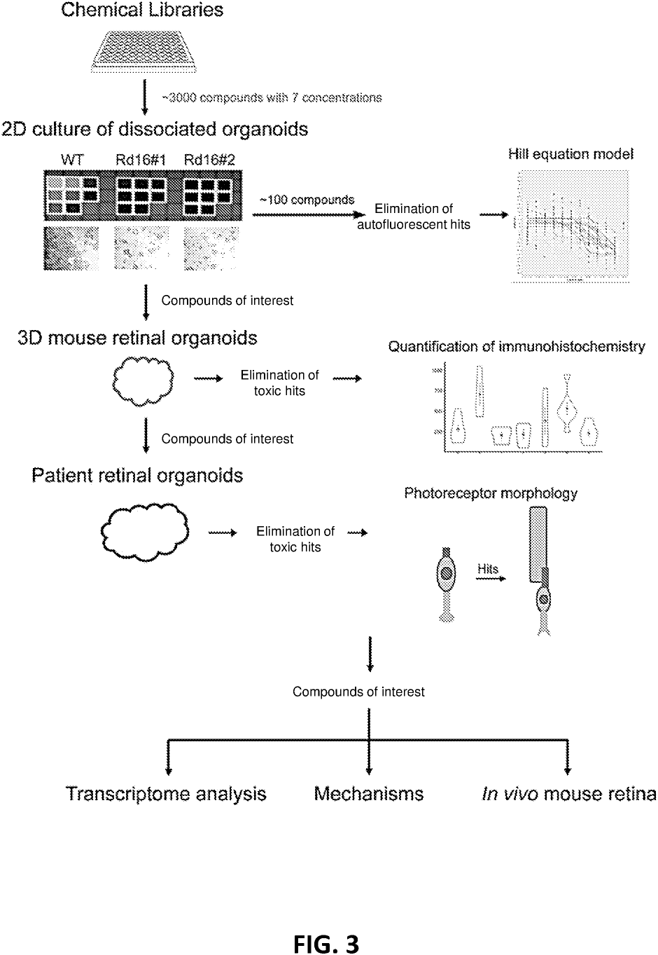

High-throughput phenotypic screening in mouse retinal organoids identified compound embodiments that maintain rod photoreceptor survival - A representative method for identifying compound embodiments of the present disclosure as compounds useful for treating retinal degeneration, particularly for treating retinal ciliopathies (including those associated with CEP290 defects), is shown by FIG. 3. As pathogenic mechanisms of CEP290-associated diseases are largely unclear, it was decided to perform untargeted high-throughput screening (HTS) to identify compound embodiments to maintain photoreceptor survival. Due to technical challenges, human iPSC differentiation into retinal organoids could hardly meet the large-scale demand of cells in HTS. As cilia biogenesis is largely conserved between mice and human (Soares et al., 2 Cells, 8, 2019), a multiplexed HTS platform was set up using retinal organoids derived from iPSCs of Nrl-GFP rd16 mouse (a model of CEP290-LCA, (Chang et al., Hum Mol Genet, 15, 1847-57, 2006)). These organoids could be generated from iPSCs efficiently with comparatively much shorter differentiation time (Chen et al., Mol Vis, 22, 1077-1094, 2016). The GFP tag under the control of the promoter of Nrl, which is the first postmitotic marker of rod photoreceptors (Akimoto et al., Proc Natl Acad Sci USA, 103, 3890-5, 2006), provided a tool to monitor rod cell biogenesis in organoid cultures. Based on the >30% lower GFP+ cells and >50% lower viability in Nrl-GFP rd16 iPSC-derived retinal organoids, a compound discovery pipeline was developed to maintain rod photoreceptor viability through screens to identify compound embodiments to increase the fluorescence intensity of GFP and nuclei stain 4′,6-diamidino-2-phenylindole (DAPI), followed by validation of the hits in mouse retinal organoids. The hits were further confirmed by transcriptome analysis, subject iPSC-derived retinal organoids, and rd16 mouse retina in vivo (FIG. 3).

In the primary screens, rd16 retinal organoids at D26, when photoreceptor cilia started to grow and abnormal phenotypes could be observed, were dissociated into single cells (GFP+ cells representing rod photoreceptors) and plated at a density of 4,000 cells/well of 1,536-well plates. D30 retinal organoids were also plated as positive control. After 24 hours, approximately 6000 small molecules from libraries of Sigma LOPAC, FDA-approved drugs, and agonists and antagonists of major cellular signaling pathways were applied to the cells at 7 different concentrations, with DMSO (solvent for small molecules) as control. After 48-hour incubation, the treated cells were fixed and stained with DAPI. By gating with the untreated group, approximately 100 compounds seemed to show positive effects on GFP and DAPI signal intensity. To remove false positive hits due to the autofluorescence of compounds, these initial hits from primary screens were applied to dissociated D26 organoids differentiated from parental PSC-derived organoids, which do not harbor GFP marker. Compounds with high autofluorescence signal were then eliminated from subsequent experiments. After normalization with DMSO control, 14 compound embodiments were selected based on their potency that was calculated as the concentration of half-maximal activity derived from the Hill equation model.

### Example 8

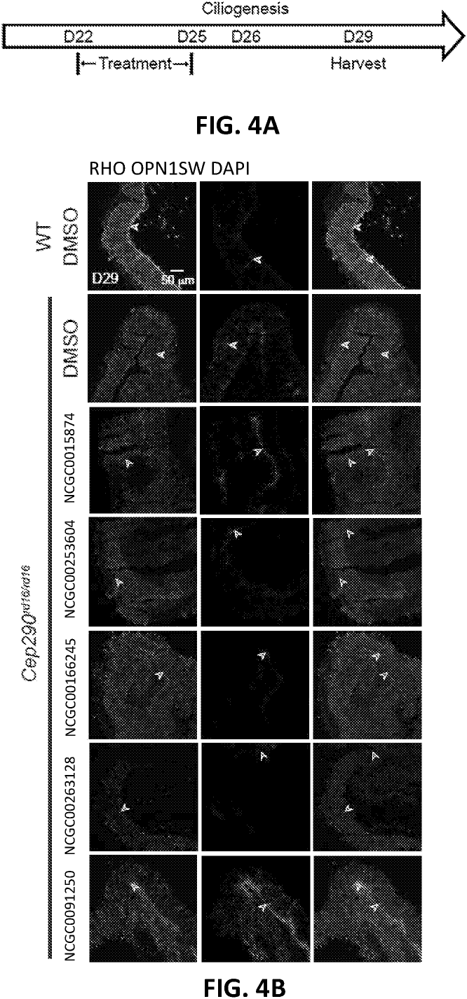

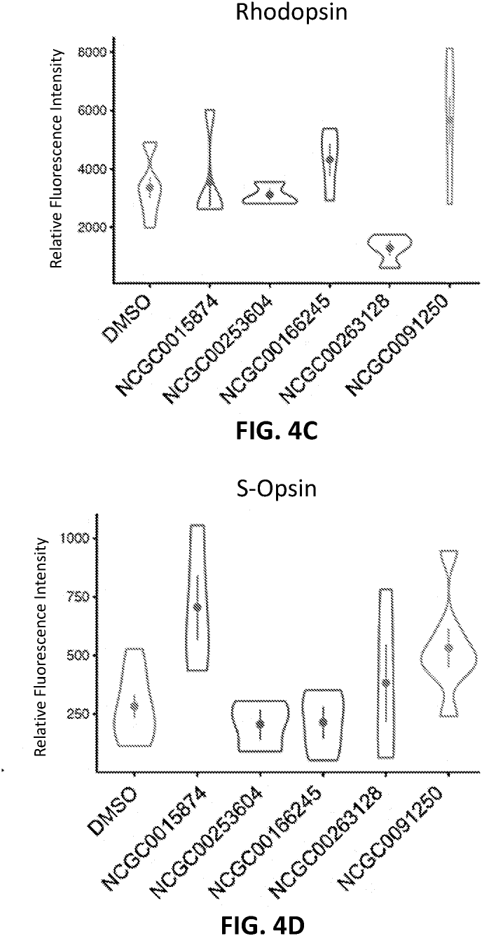

Rhodopsin and S-opsin expression were increased in rd16 retinal organoids treated with compound embodiments - The 14 compound embodiments were then tested with intact rd16 retinal organoid cultures at AC50 and half of AC50 to evaluate their toxicity and effect. Small molecules leading to dissociation of retinal organoids or photoreceptor death at 0.5x AC 50 would be removed from subsequent validation. The compounds were applied directly to the cultures at D22 and removed at D25. Treated organoids were harvested 72 hours after removal of compounds at D28 (FIG. 4A). Five compound embodiments (NCGC0091250, Reserpine; NCGC00253604, Rescimetol; NCGC00263128, CHEMBL39740; NCGC00015874, Quinacrine dihydrochloride dihydrate; NCGC00166245, Proflavine hemisulfate) demonstrated higher immunostaining of markers for rod and/cone photoreceptors in rd16 organoid cultures. As shown in FIG. 4B, immunostaining of rhodopsin in untreated rd16 photoreceptors is very faint, with loss of polarity at the apical side of neural retina. Treatment with these compound embodiments improved expression and polarity of rhodopsin, with variable potency. Notably, although cone photoreceptor biogenesis was compromised even in WT organoids at D28, some compound embodiments, NCGC0091250 for example, were able to increase the expression and polarization of S-opsin in cone photoreceptors, suggesting a favorable effect on S-cones as well. To account for high variability of mouse retinal organoids, the fluorescence intensity of rhodopsin and S-opsin staining in all untreated and treated neural retina (FIG. 4C) was quantified using an imaging algorithm that captured most pixels and avoided background in immunostaining. The improvement was confirmed with selected compound embodiments on rod and/or cone photoreceptors in rd16 retinal organoids. NCGC00253604 is a derivative of NCGC0091250, but it is not as potent as NCGC0091250 showing only borderline improvement of rhodopsin staining in mouse retinal organoids.

### Example 9

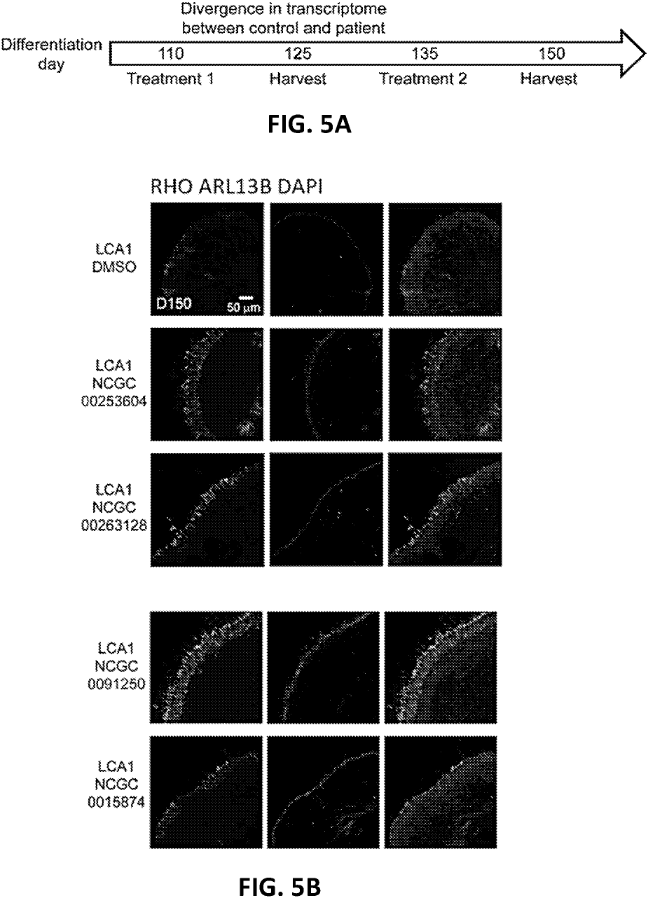

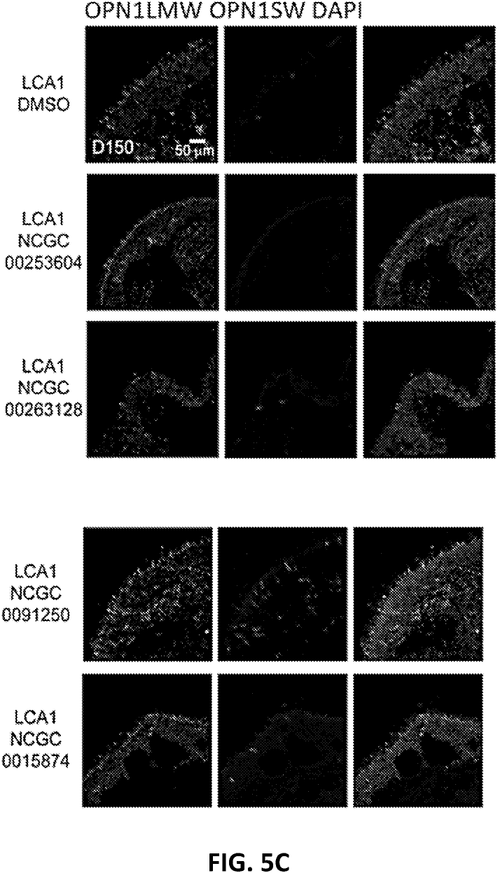

Subject iPSC-derived retinal organoids showed improvement in photoreceptor biogenesis after treatment with compound embodiments - To further validate the five compound embodiments, LCA subject iPSCs were differentiated into retinal organoids and treated with the selected small molecules. Comparative transcriptome analysis of gene profiles of control and subject retinal organoids indicated that the most dramatic divergence was observed at D120 (FIG. 2D). Therefore, drug treatments were applied at D110 and D135, each of which lasted for 3 days, and retinal organoids were harvested at D125 and D150 for evaluation of photoreceptor and cilia biogenesis by immunostaining (FIG. 5A). Due to the different sensitivity of small molecules and the reversed configuration of neural retina between mouse and human retinal organoids, 5-40 µM of each compound was re-evaluated in subject organoids. One compound embodiment, NCGC00166245, demonstrated toxicity in organoids of one of the subjects within this range and was removed from further validation experiments. The remaining 4 were applied to subject organoid cultures. D150 subject organoids had barely detectable rhodopsin and limited development of ciliary axoneme, which were improved by treatment of different small molecules (FIG. 5B). Although cone photoreceptors were not dramatically impacted in subject organoids, the improvement of cone cells was noted in subject organoids with two small molecule treatments (NCGC0091250, NCGC0015874), which is consistent with their effects on mouse organoids (FIG. 5C). Treatment of NCGC00253604, a derivative of NCGC009125, demonstrated a more potent effects on human rod photoreceptors compared to mouse ones.

### Example 10

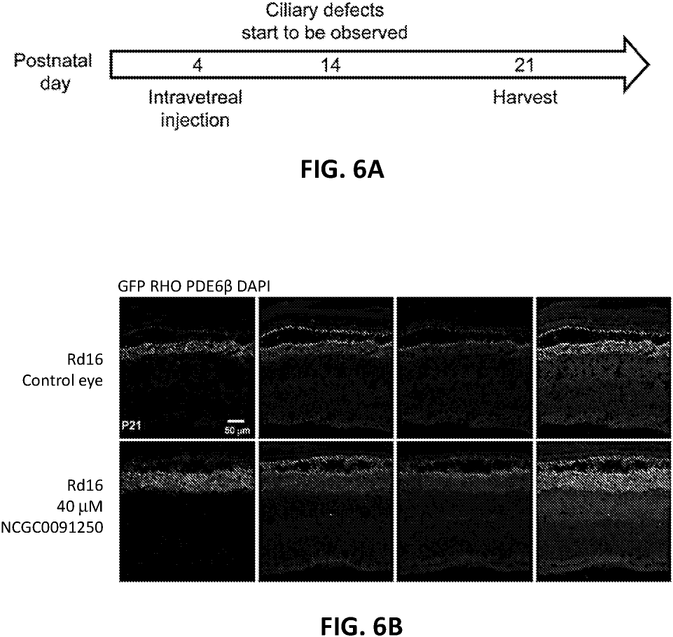

Intravitreal injection of compound embodiments into rd16 mouse maintained the thickness of the outer nuclear layer of photoreceptors - To verify the compound embodiments in vivo, intravitreal injection was preformed, to deliver the compounds into Nrl-GFP rd16 mouse retina and assess the survival of photoreceptors in the outer nuclear layer (ONL). As differences between wildtype and rd16 mouse retina appear as early as postnatal day (P) 6, the compound was delivered intravitreally at P4, with one eye receiving DMSO (control) and the other eye candidate compounds. The eyes were harvested at P21 (FIG. 6A). To systematically assess technical issues including injection techniques, compound concentration and toxicity, the experiment was started with one compound NCGC0091250, which revealed the most significant effect in mouse and human organoids. In 2 out of the 3 injected animals, injection of 40 µ M NCGC0091250 maintained the thickness of ONL at P21, as shown by GFP (rod cells) and DAPI (FIG. 6B), compared to the control eye without treatment. Photoreceptor ciliary proteins, including rod-specific proteins rhodopsin (RHO) and cyclic GMP phosphodiesterase β (PDE6β), were transported to the outer segment region. Consistently, treated retina had more ciliary proteins located at the longer outer segments compared to the untreated ones. In all three injected mice, no obvious toxicity was observed.

### Example 11

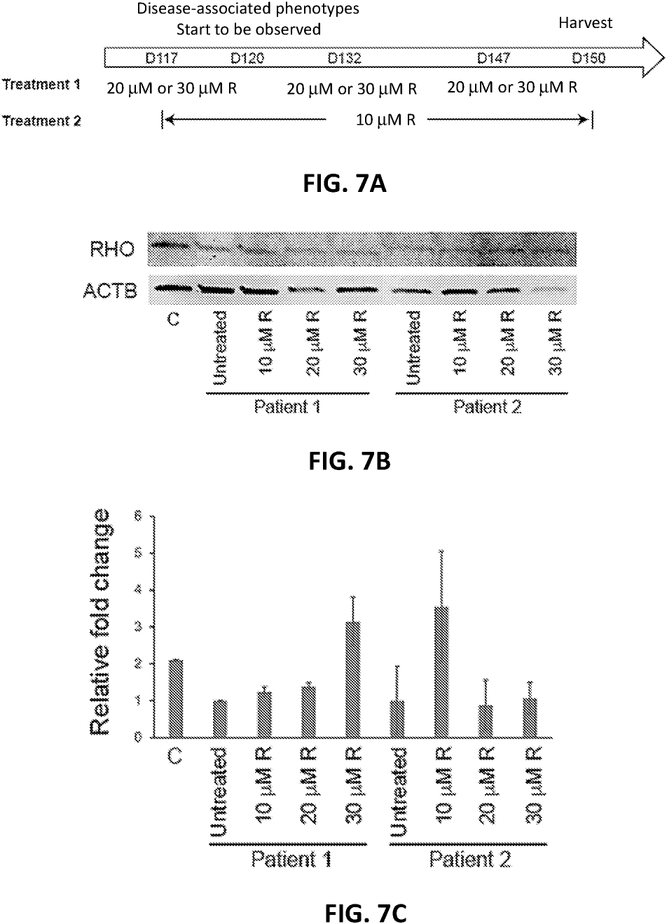

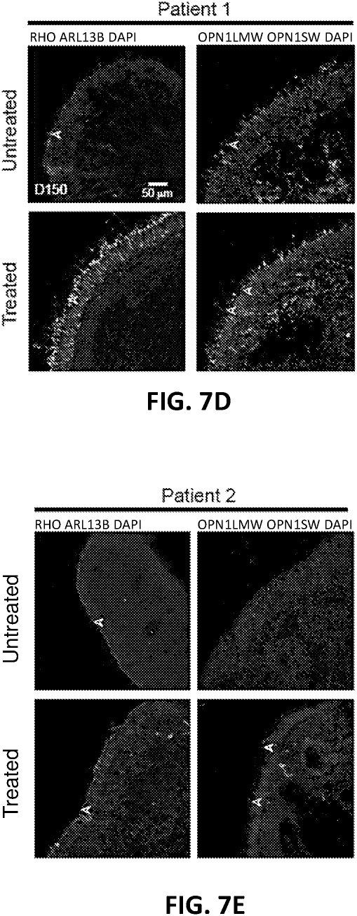

Evaluation of the drug effect on subject retinal organoids - The timeline for the drug treatment on CEP290-LCA (IVS26+1655A>G p.C998X; c.5668G>T p.G1890X) is show in FIG. 7A. Subject induced pluripotent stem cells (iPSC)-derived retinal organoids were used and two treatment modules were evaluated: (1) intermittent high dose (20 µM and 30 µM); and (2) continuous low dose (10 µM). The treatments started 3 days before abnormal phenotypes in subject organoids could be observed (D117) and the organoids were harvested at D150 for analyses. The drug vehicle DMSO was added as control with a concentration (v/v) less than 1%. FIGS. 7B and 7C show the Western blot analyses and quantification rhodopsin level in subject organoids, respectively. The data are presented as mean ± standard deviation from 2 batches of experiments, each of which had at least 2 retinal organoids. Beta-actin (ACTB) were used as a loading control. As determined from the data, retinal organoids from both subjects harbored a lower expression of rhodopsin compared to those from the familial control (labeled as “C” in FIGS. 7B and 7C), suggesting defects in rod photoreceptors. Treatments of different concentrations of reserpine (labeled as “R” in FIGS. 7A-7C) were able to improve rhodopsin staining. In this example, 30 µM reserpine exhibited a positive effect in subject 1, whereas 10 µM was sufficient for subject 2, possibly indicating variations in subjects or cell lines. The images shown by FIGS. 7D and 7E confirmed the results of the Western blot that reserpine showed improved both photoreceptors and ciliary axoneme in subject organoids.

### Example 12

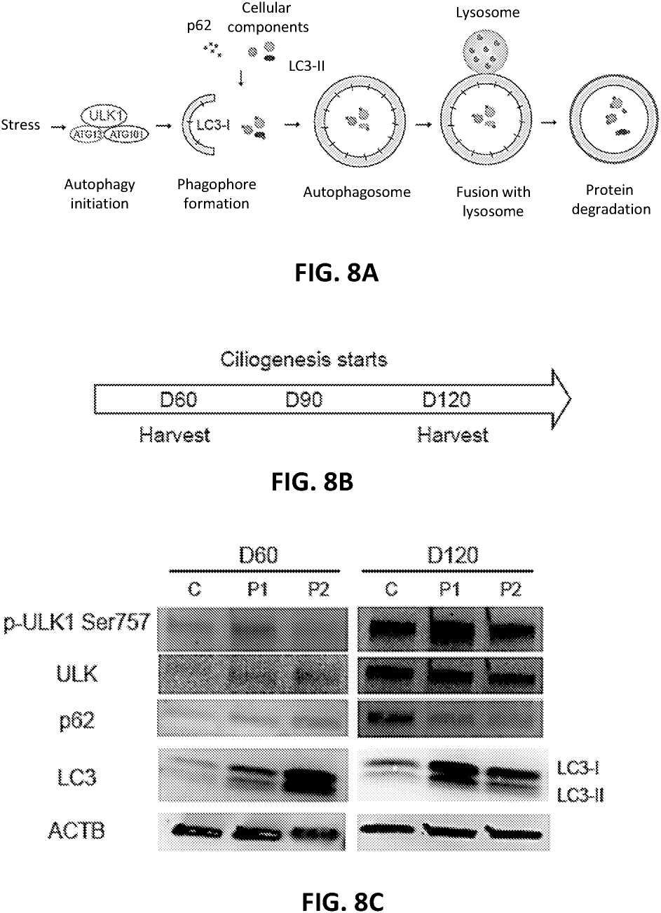

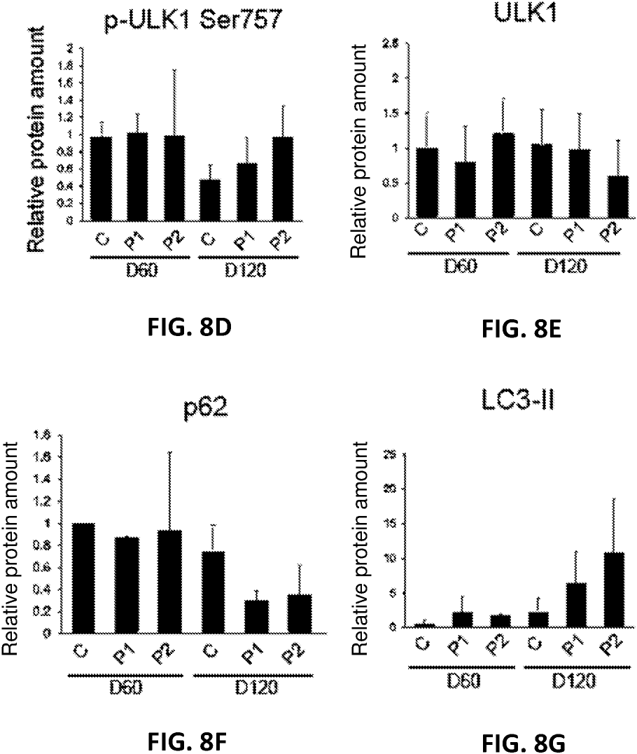

Evaluation of misregulation of autophagy in subject organoids - As the common pathway of all the positive hits in the rd16 organoids mouse was autophagy inhibition, the autophagy level in subject retinal organoids was assessed. Autophagy is a cellular homeostatic mechanism whose initiation could be induced by stress, leading to phosphorylation of ULK1 (see FIG. 8A). Together with other autophagy components ATG101 and ATG13, p-ULK1 triggers the formation of phagophore, which is an extension of the endoplasmic reticulum membrane. A key autophagy adaptor p62 binds to ubiquitinated cellular components and delivers them to phagophores to form a sealed vesicles termed autophagosome. LC3-II, a standard marker for autophagosomes, is generated by conjugation of cytosolic LC3-I to phosphatidylethanolamine (PE) on the surface of nascent autophagosomes LC3-II. Cellular components in the autophagosome are degraded by fusion with lysosomes. To evaluate the overall autophagic status in subject organoids, several components in the process, including p-ULK1, ULK1, p62 and LC3, were evaluated. FIG. 8B summarizes the timeline used for the evaluations in this example. As cilium biogenesis and photoreceptor maturation start at around D90, control and subject organoids were harvested at D60 and D120 to evaluate the impact of ciliary defects on cellular autophagy. FIGS. 8C and 8D-8G show Western blot analyses and autophagy component quantification in subject organoids, respectively (the data are presented as mean ± standard deviation from 2 batches of experiments, each of which had at least 3 retinal organoids); beta-actin (ACTB) were used as a loading control. At D60, no significant difference was found in the tested autophagy components between control and subject organoids; however, an augmented autophagy initiation could be observed in subject organoids, as shown by upregulation of p-ULK1. A significantly down-regulation of p62 and up-regulation of LC3-II consistently indicated misregulation of the autophagic flux in subject organoids compared to the control.

### Example 13

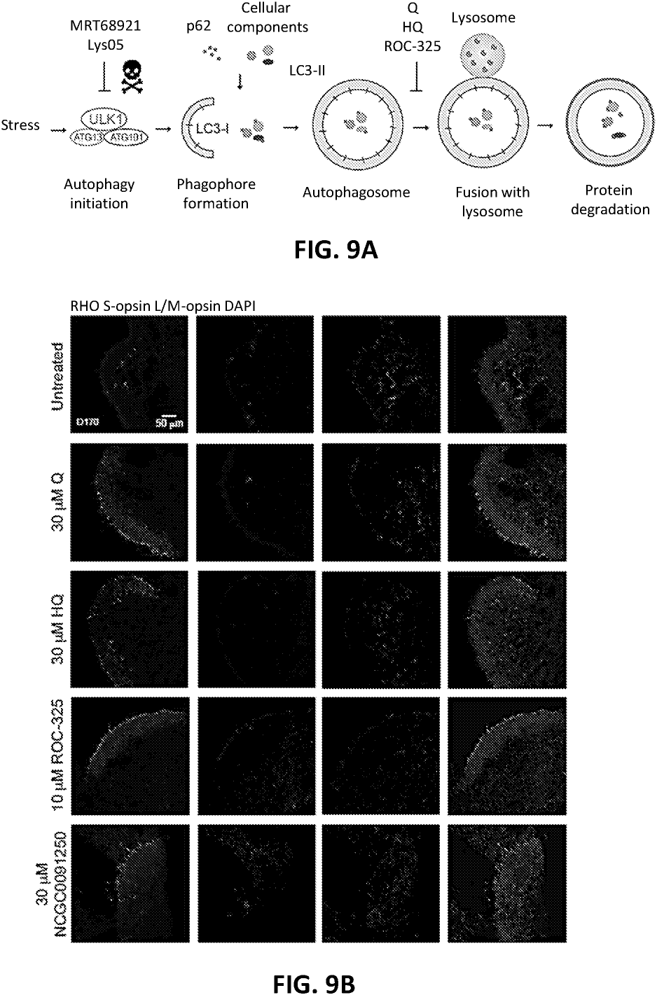

Drug repurposing of autophagy inhibitors - To confirm the effect of autophagy inhibition on rescuing subject photoreceptors and to identify key autophagic molecule(s) involved in this process, various FDA-approved autophagy inhibitor drugs were applied on organoid cultures at their reported AC₅₀ and 2x AC₅₀ (summarized by FIG. 9A). MRT68921 and Lys05 inhibit phosphorylation of ULK1. Chloroquine (Q), hydroxychloroquine (HQ) and ROC-325 increase the pH of lysosomes to prevent their fusion with autophagosomes. MRT68921 and Lys05 exhibited high toxicity even at 0.5x AC50 (data not shown) and thus were omitted in subsequent analyses. FIG. 9B shows the results from immunostaining of rod (rhodopsin, green), S-cone (S-opsin, red) and L/M-cone (L/M-opsin, magenta) photoreceptors. The immunostaining analyses revealed a positive effect of all autophagy inhibitors on subject photoreceptors, although with various efficacies, suggesting that autophagy inhibition plays a role in maintenance/improvement of photoreceptors in retinal degenerative diseases.

### Example 14

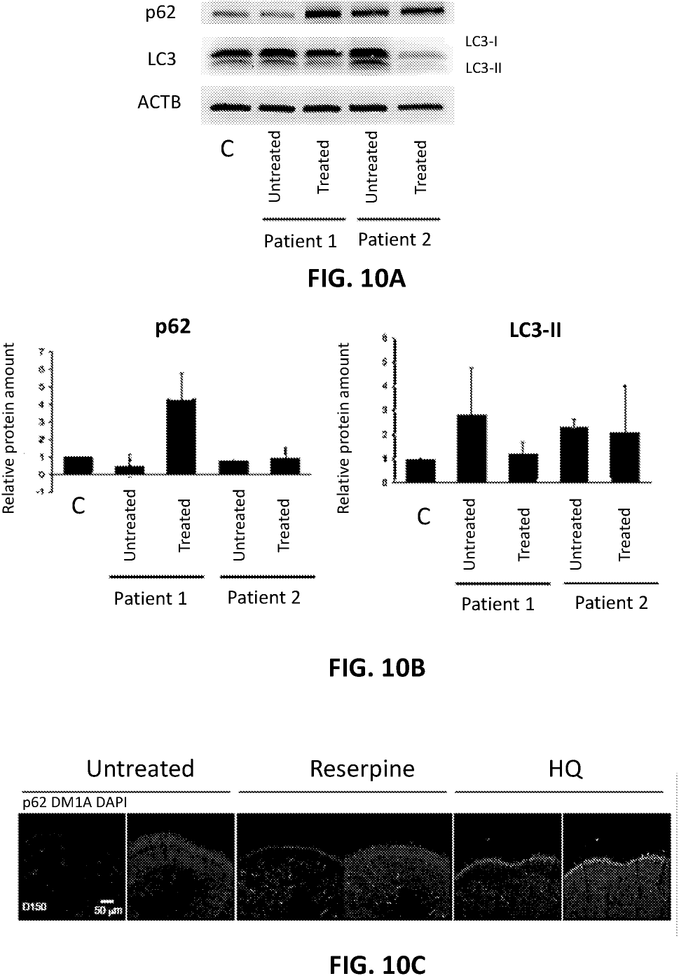

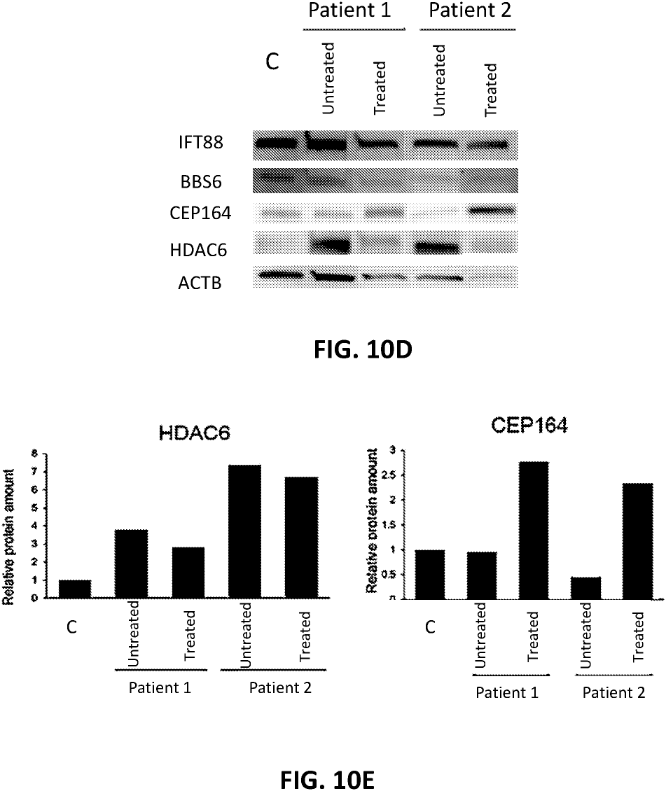

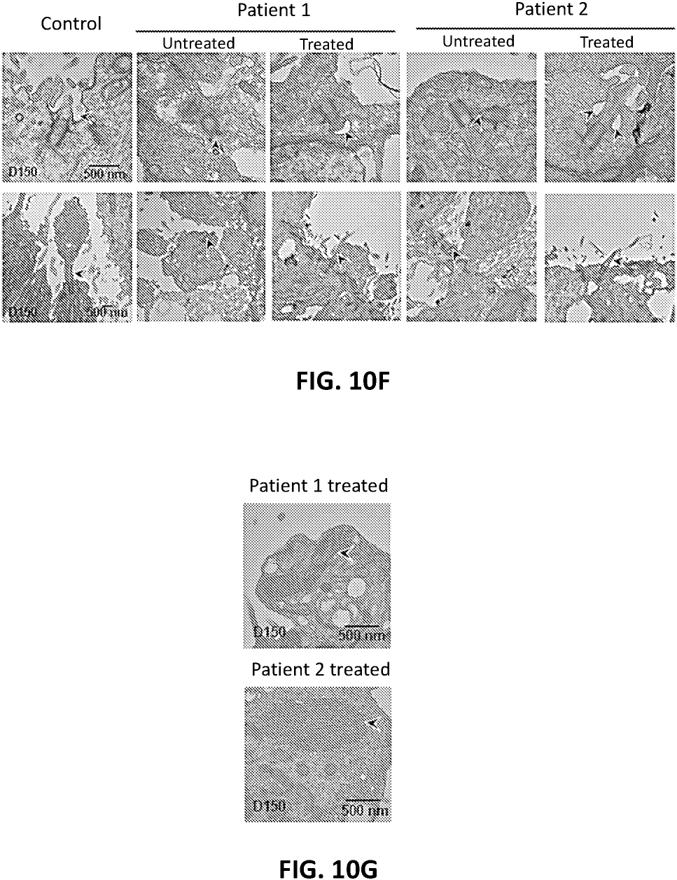

p62 mediation - In this example, the increase of p62 by reserpine in treated subject organoids was evaluated. FIGS. 10A and 10B show Western blot analyses and p62 and LC3-II quantification, respectively. As can be seen in FIG. 10B, LC3-II level decreased in one subject but not the other one. Notably, a more significant change of p62 was observed in the subject more responsive to reserpine treatment. FIG. 10C show the results from immunostaining of p62 and acetylated tubulin (DM1T) in treated subject organoids, which were performed to confirm an increase of p62 in photoreceptors in subject organoids treated by reserpine and hydroxychloroquine (HQ). DM1T staining also indicated more well developed ciliary axoneme in treated subject photoreceptors. FIGS. 10D and 10E show Western blot analyses and quantification of p62 interaction partner and cilium disassembly key driver, HDAC6, and other ciliary regulatory proteins, including IFT88 (intraflagellar transport), BBS6 and CEP164 (distal appendage component for initiation of ciliogenesis) in treated organoids. Down-regulation of HDAC6 and up-regulation of CEP164 were observed in subject organoids treated with reserpine. As HDAC6 is a major driver for cilium biogenesis and CEP164 is located in distal appendage of docking of preciliary vesicles for initiation of ciliogenesis, transmission electron microscopy (TEM) was performed to uncover more details of photoreceptors in untreated and treated subject organoids. Defects in docking of preciliary vesicles and formation of ciliary membrane have been reported to be early phenotypes in CEP290-LCA subject retinal organoids, and such defects could be alleviated by treatment of reserpine (see FIG. 10F, upper panel). TEM analyses also revealed longer ciliary axoneme in treated photoreceptors (see FIG. 10F, lower panel). Notably, a well-organized disc-like structure, which is rare in organoid culture, could be observed in subject organoids (see FIG. 10G), suggesting a favorable effect of reserpine on the development of outer segment (primary cilium of photoreceptors).

### Example 15

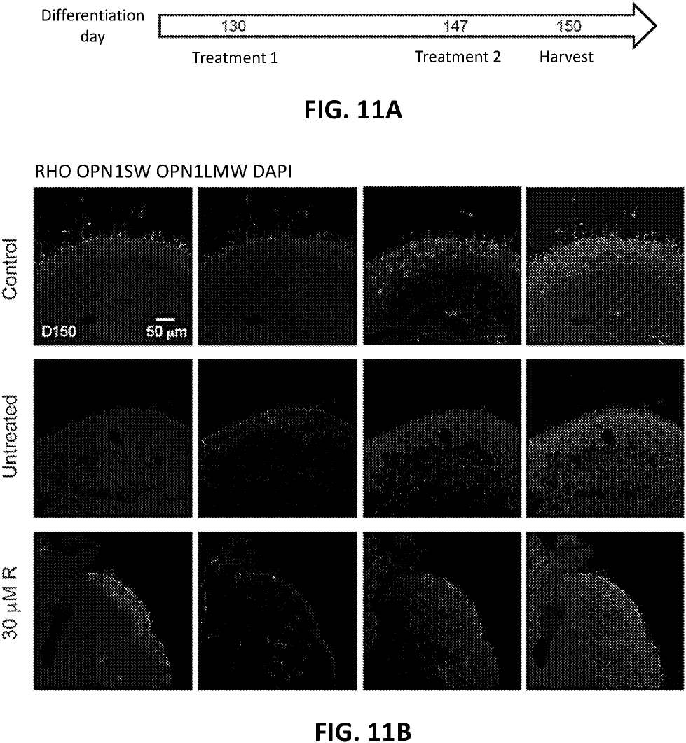

Improved photoreceptor morphology after short-term treatment of CEP290-LCA subject induced pluripotent stem cell (iPSC)-derived retinal organoids - To evaluate the effect of reserpine on subject organoids caused by a different mutation, short-term treatment of reserpine on CEP290-LCA subject organoids caused by homozygous IVS26+1655A>G p.C998X, which is the most common mutations of CEP290-LCA, was performed. FIG. 11A provides a schematic diagram showing the small molecule treatment paradigm for CEP290-LCA retinal organoids used for this example. FIG. 11B shows the images obtained from immunostaining of rod cells (green), S-cones (red) and L/M-cones (magenta). The images confirm that CEP290-LCA retinal organoids homozygous for IVS26+1655A>G p.C998X displayed defects in photoreceptor development and treatment of reserpine was able to improve rod photoreceptors in cultures.

In view of the many possible embodiments to which the principles of the present disclosure may be applied, it should be recognized that the illustrated embodiments are only preferred examples and should not be taken as limiting the scope of the disclosure. Rather, the scope is defined by the following claims. We therefore claim as our invention all that comes within the scope and spirit of these claims.

We claim:

1. A method of treating retinal degeneration in a subject, comprising administering to the subject a therapeutically effective amount of a compound thereby treating the retinal degeneration in the subject, wherein the compound is selected from a compound having a structure according to a formula selected from Formula I, II, or III 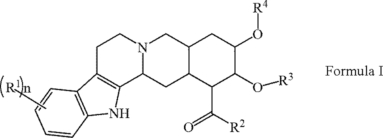 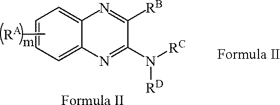 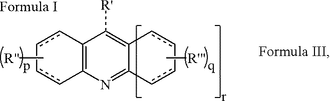
or a pharmaceutically acceptable salt, prodrug, solvate, hydrate, or tautomer thereof; or 3-(dibutylamino)-1-(1,3-dichloro-6-(trifluoromethyl)phenanthren-9-yl)propan-1-ol hydrochloride or another pharmaceutically acceptable salt, or a prodrug, solvate, hydrate, or tautomer thereof; wherein,
(i) with reference to Formula I,
R¹ is heteroaliphatic;
R² is OR⁵, or NR⁶R⁷, wherein each of R⁵, R⁶, and R⁷ independently is selected from aliphatic, hydrogen, aromatic, or an organic functional group;
R³ is selected from aliphatic, aromatic, acyl, or sulfonyl;
R⁴ is selected from acyl, aliphatic, aromatic, or sulfonyl; and
n is an integer selected from 0 to 4;
(ii) with reference to Formula II,
R^{A}, is selected from halogen, heteroaliphatic, haloaliphatic, or an organic functional group;
R^{B} is aromatic; and
each of R^{C} and R^{D} independently is selected from hydrogen, aliphatic, or heteroaliphatic; and
m is an integer selected from 0 to 4; and
(iii) with reference to Formula III,
R′ is selected from aliphatic, aromatic, halogen, heteroaliphatic, haloaliphatic, or an organic functional group;
each R″ independently is selected from halogen, heteroaliphatic, or amino;
each R‴ independently is selected from halogen, heteroaliphatic, or amino;
p is an integer selected from 0 to 4;
q is an integer selected from 0 to 4; and
r is an integer selected from 0 or 1.

2. The method of claim 1, wherein the subject has retinitis pigmentosa, LCA, Stargardt’s macular dystrophy, cone-rod dystrophy, choroideremia or age-related macular degeneration.

3. The method of claim 1, wherein the compound is administered orally or locally to the eye of the subject.

4. (canceled)

5. The method of claim 3, wherein the compound is administered intravitreally in the eye of the subject.

6. The method of claim 1, wherein the subject is human.

7. The method of claim 1, wherein with the compound (i) maintains thickness of a nuclear layer of photoreceptors in a retina of the eye of the subject, (ii) increases expression of an opsin in the retina of the subject, (iii) increases the number of photoreceptor cells in the subject, or a combination of (i), (ii), and/or (iii).

8. (canceled)

9. The method of claim 7, wherein the opsin is a cone opsin, rhodopsin, or a phototransduction protein that comprises rod cyclic GMP phosphodiesterase 6β (PDE6β).

10. (canceled)

11. The method of claim 1, further comprising evaluating the vision of the subject by performing electroretinography on the subject.

12. (canceled)

13. The method of claim 1, wherein the compound has a structure according to any one of Formulas IA, IC, or IE, or a pharmaceutically acceptable salt, prodrug, solvate, hydrate, or tautomer thereof 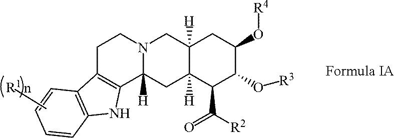 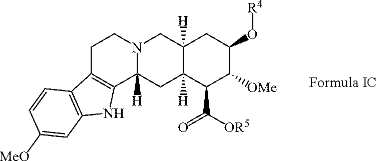 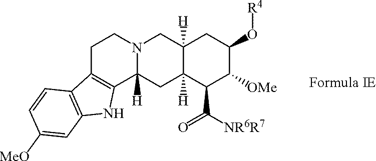
.

14. The method of claim 1, wherein the compound has a structure according to Formula I, or a structure according to Formulas IA, IC, or IE, or a pharmaceutically acceptable salt, prodrug, solvate, hydrate, or tautomer thereof, 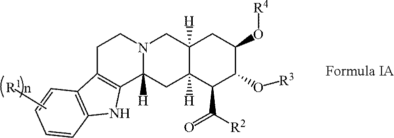 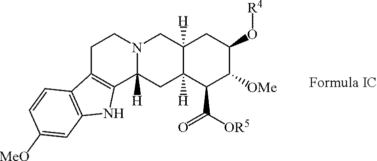 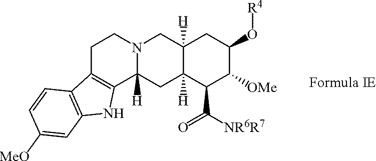
wherein:
R¹ is alkoxy;
R² is -OR⁵, or -NR⁶R⁷, wherein each of R⁵, R⁶, and R⁷ independently is selected from alkyl, hydrogen, heteroaryl, or aryl;
R³ and is selected from alkyl, heteroaryl, aryl, sulfonyl, or acyl;
R⁴ is selected from acyl, alkyl, heteroaryl, aryl, or sulfonyl; and
n is 0, 1, 2, 3, or 4.

15. The method of claim 1, wherein the compound has a structure according to Formula I, or a structure according to Formula IA, IC, or IE, or a pharmaceutically acceptable salt, prodrug, solvate, hydrate, or tautomer thereof, 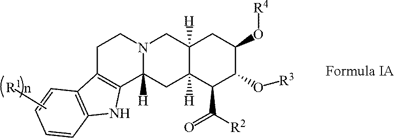 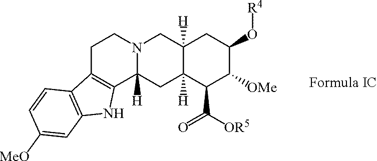 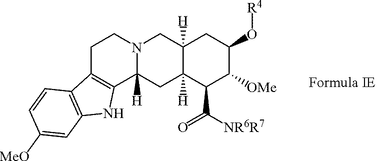
and wherein R⁴ is selected from 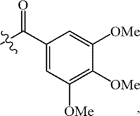 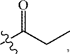 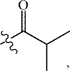 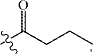 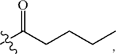 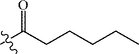 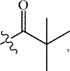 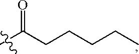 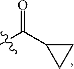 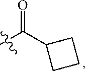 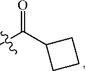 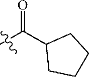 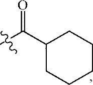 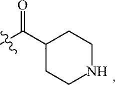 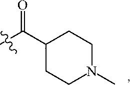 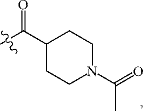 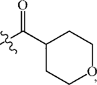 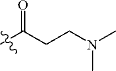 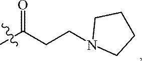 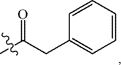 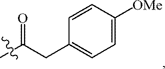 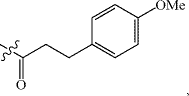 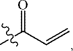 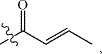 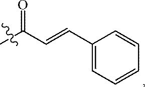 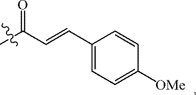 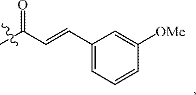 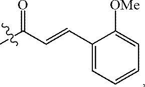 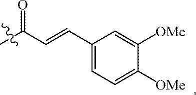 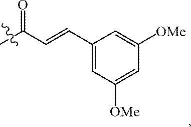 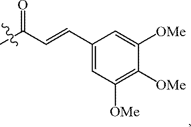 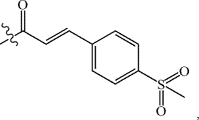 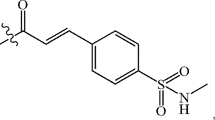 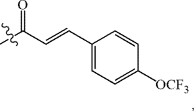 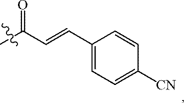 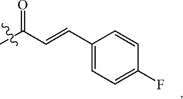 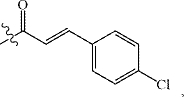 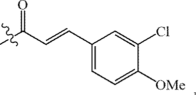 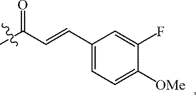 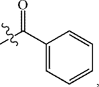 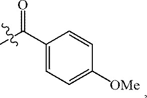 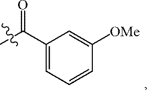 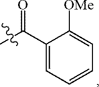 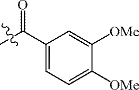 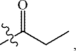 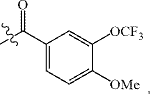 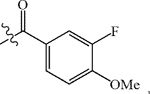 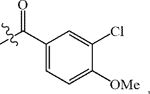 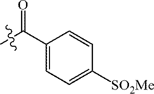 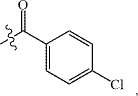 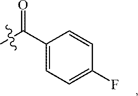 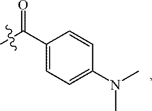 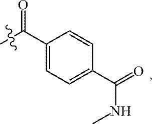 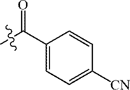 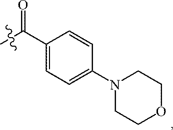 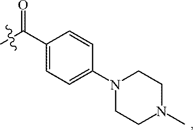 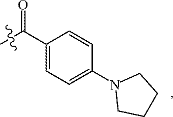 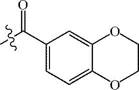 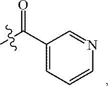 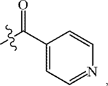 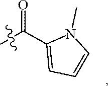 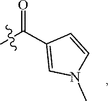 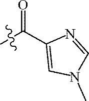 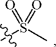 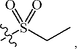 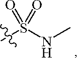 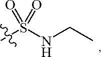 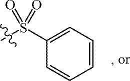 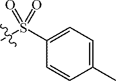
.

16. The method of claim 1, wherein the compound is selected from 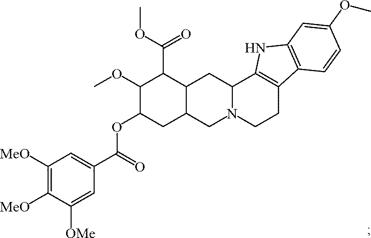 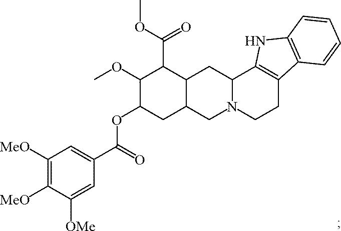 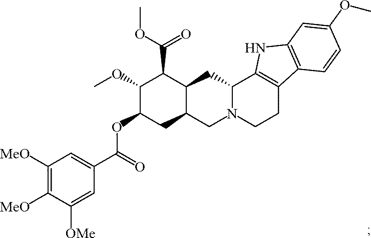 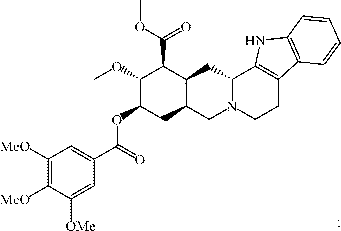 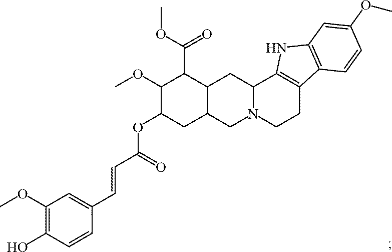 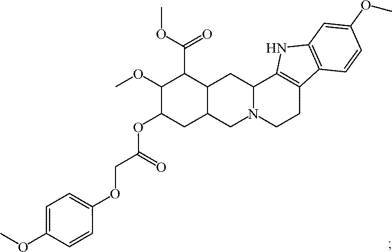 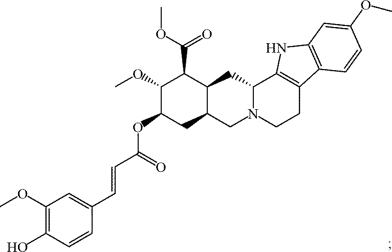 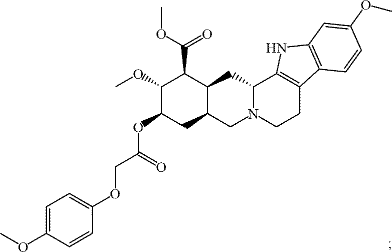 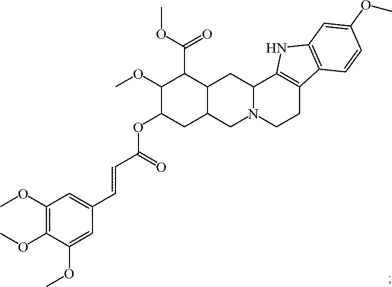 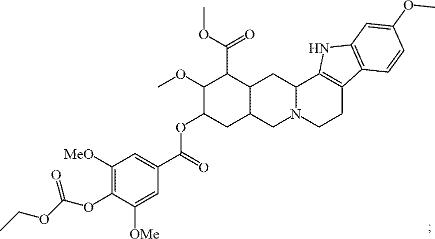 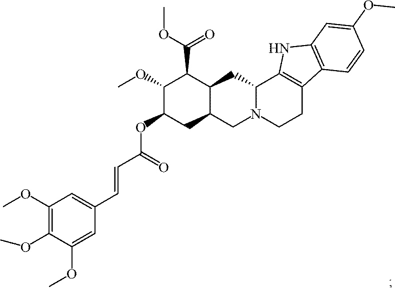 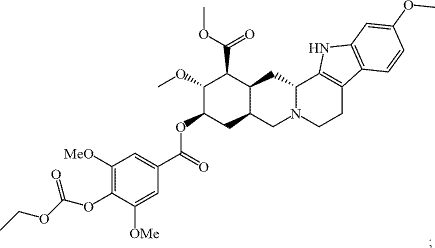 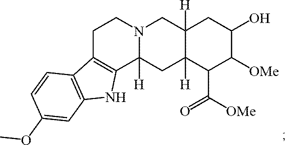 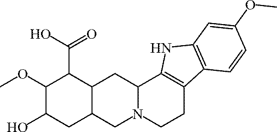 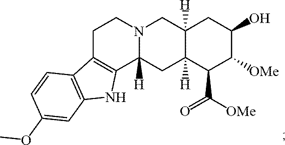 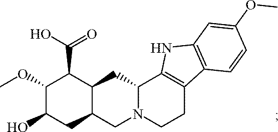
or a pharmaceutically acceptable salt, prodrug, solvate, hydrate, or tautomer thereof.

17. The method of claim 1, wherein the compound has a structure according to Formula IIA, or a pharmaceutically acceptable salt, prodrug, solvate, hydrate, or tautomer thereof

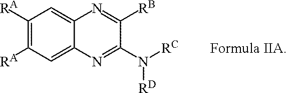

18. The method of claim 1, wherein the compound has a structure according to Formula II or a structure according to Formula IIA, or a pharmaceutically acceptable salt, prodrug, solvate, hydrate, or tautomer thereof, 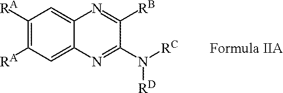
wherein:
each R^{A} independently is selected from halogen, —OMe, —CN, or —CF₃;
R^{B} is selected from aryl; aryl comprising one or more substituents selected from halogen, —CF₃, —CN, —OH, alkyl, or alkoxy; heteroaryl; heteroaryl comprising one or more substituents selected from halogen, —CF₃, —CN, —OH, alkyl, or alkoxy;
each of R^{C} and R^{D} independently is selected from hydrogen, alkyl, or amino; and
m is 0, 1, 2, 3, or 4.

19. The method according to claim 1, wherein the compound has a structure according to Formula II or a structure according to Formula IIA, or a pharmaceutically acceptable salt, prodrug, solvate, hydrate, or tautomer thereof, 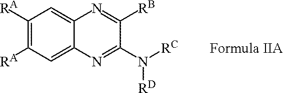
and R^{B} is selected from 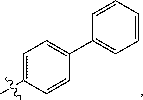 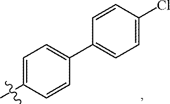 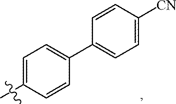 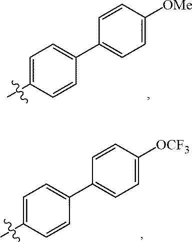 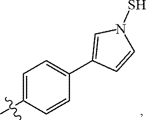 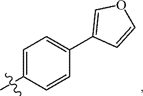 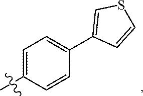 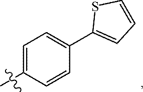 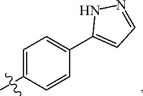 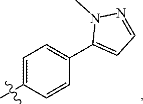 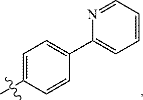 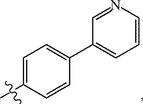 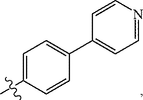 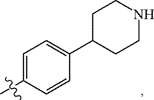 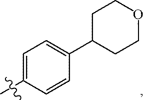 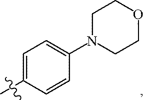 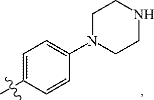 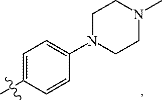 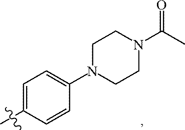 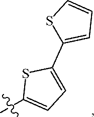 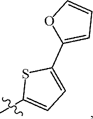 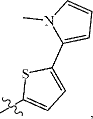 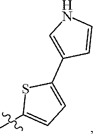 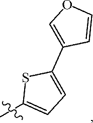 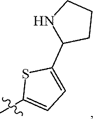 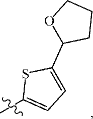 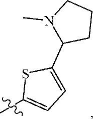 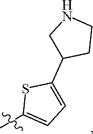 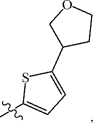 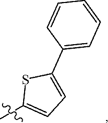 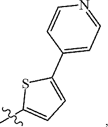 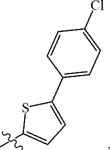 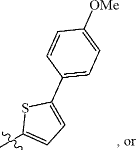 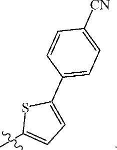
.

20. The method according to claim 1, wherein the compound is 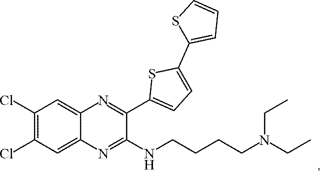
or a pharmaceutically acceptable salt, prodrug, solvate, hydrate, or tautomer thereof.

21. The method according to claim 1, wherein the compound has a structure according to Formulas IIIA-IIID, or a pharmaceutically acceptable salt, prodrug, solvate, hydrate, or tautomer thereof

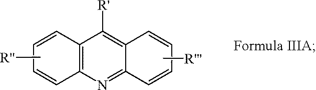

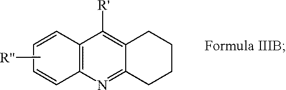

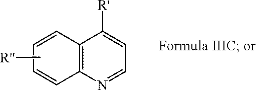

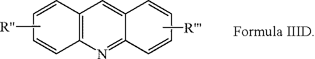

22. The method according to claim 1, wherein the compound has a structure according to Formula III or a structure according to Formulas IIIA-IIID, or a pharmaceutically acceptable salt, prodrug, solvate, hydrate, or tautomer thereof, 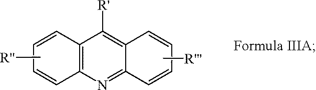 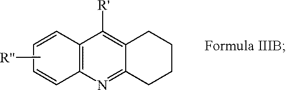 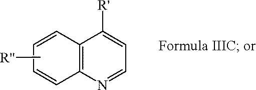 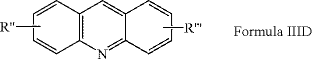
and wherein
R′ is selected from halo, —CN; —CF₃; —OCF₃; alkyl; heteroalkyl comprising one or more nitrogen atoms, one or more oxygen atoms, one or more sulfur atoms, or a combination thereof; or aminoaryl;
R″ is selected from halogen, alkoxy, or -NR^{a′}R^{b′}, wherein each of R^{a′} and R^{b′} independently is selected from alkyl, heteroalkyl, benzyl, acyl, sulfonyl;
R‴ is selected from halogen, alkoxy, or -NR^{a′}R^{b′}, wherein each of R^{a′} and R^{b′} independently is selected from alkyl, heteroalkyl, benzyl, acyl, or sulfonyl;
p and q independently is an integer selected from 0, 1, 2, 3, or 4; and
r is 0 or 1.

23. The method according to claim 1, wherein the compound has a structure according to Formula III or a structure according to Formulas IIIA-IIID, or a pharmaceutically acceptable salt, prodrug, solvate, hydrate, or tautomer thereof, 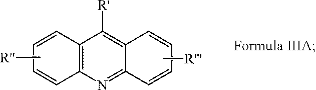 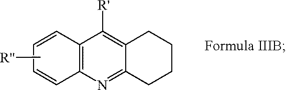 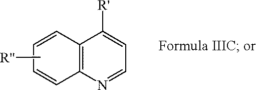 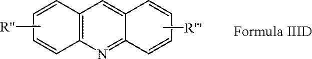
wherein R′ is selected from 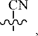 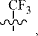 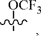 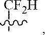 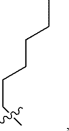 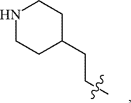 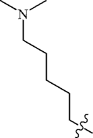 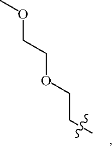 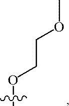 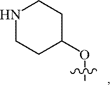 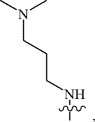 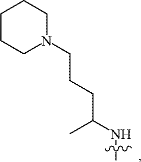 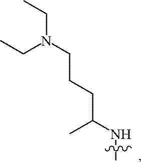 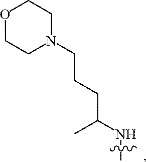 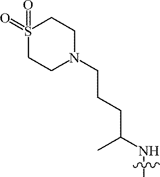 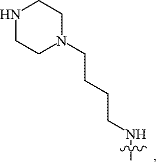 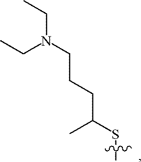 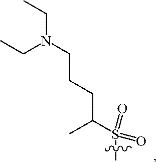 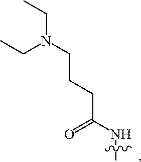 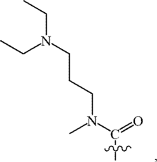 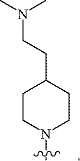 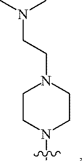 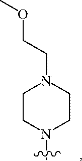 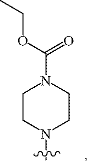 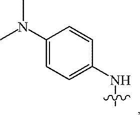 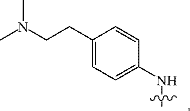 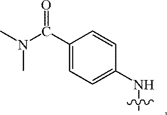 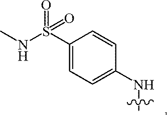 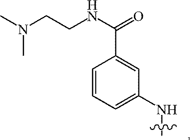 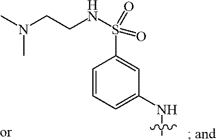
wherein R″ and R‴ independently is -NR^{a′}R^{b′}, wherein one of R^{a′} and R^{b′} is H and the other is selected from 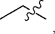 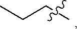 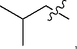 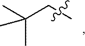 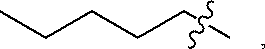 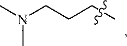 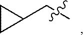 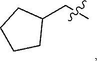 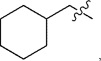 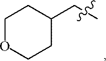 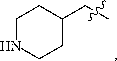 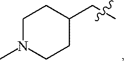 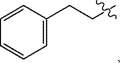 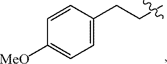 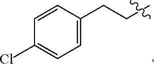 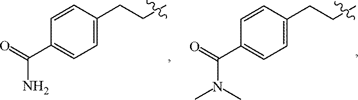 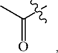 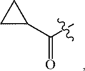 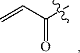 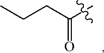 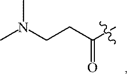 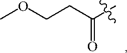 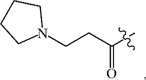 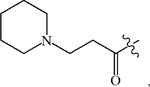 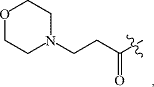 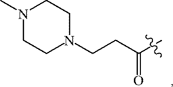 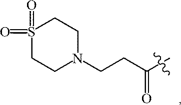 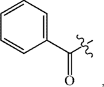 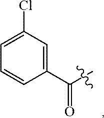 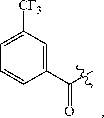 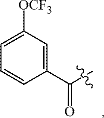 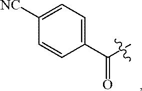 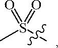 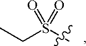 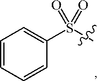 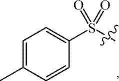 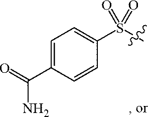 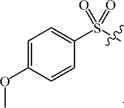
.

24. The method according to claim 1, wherein the compound is selected from 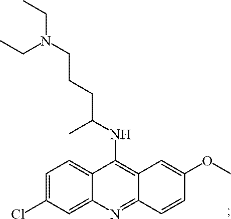 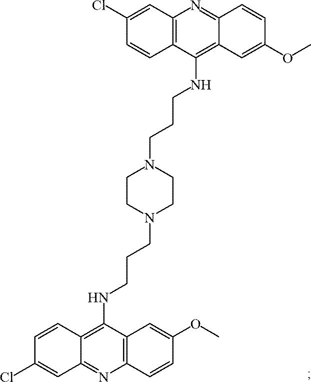 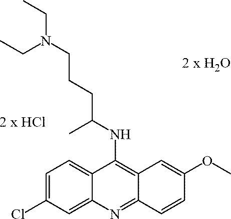 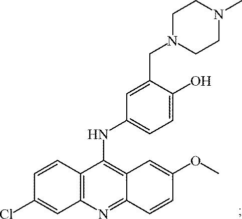 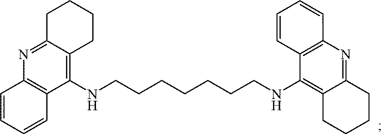 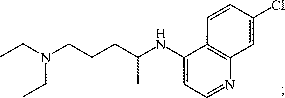 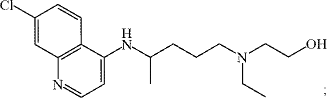 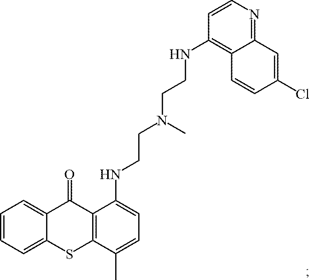 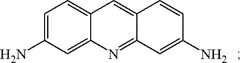 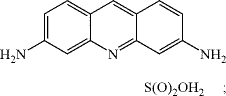 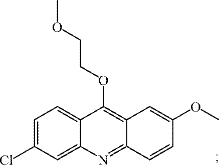 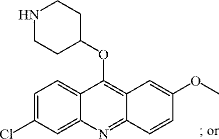
a pharmaceutically acceptable salt, prodrug, solvate, hydrate, or tautomer thereof.

25. The method of claim 1, wherein the compound is selected from 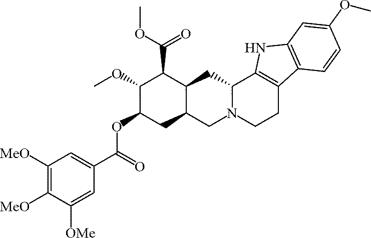 [SMILES: COC(=O)[C@H]1[C@H]2C[C@@H]3c4[nH]c5cc(OC)ccc5c4CCN3C[C@H]2C[C@@H](OC(=O)c2cc(OC)c(OC)c(OC)c2)[C@@H]1OC] 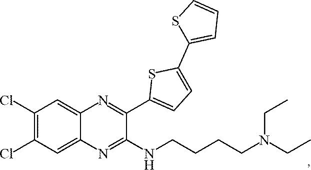 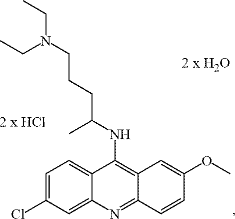 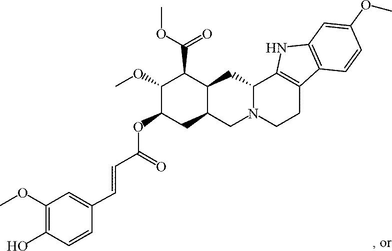 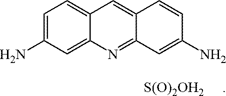
.

26. A composition, comprising:
a therapeutically effective amount of a compound having a structure according to a formula selected from Formula I, II, or III 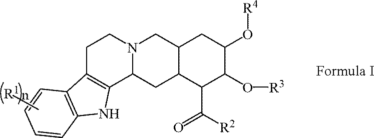 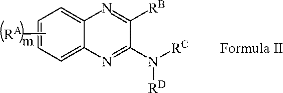 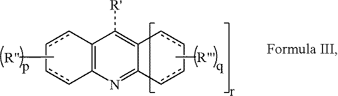
or a pharmaceutically acceptable salt, prodrug, solvate, hydrate, or tautomer thereof; or 3-(dibutylamino)-1-(1,3-dichloro-6-(trifluoromethyl)phenanthren-9-yl)propan-1-ol hydrochloride or another pharmaceutically acceptable salt, or a prodrug, solvate, hydrate, or tautomer thereof; wherein,
(i) with reference to Formula I,
R¹ is heteroaliphatic;
R² is OR⁵, or NR⁶R⁷, wherein each of R⁵, R⁶, and R⁷ independently is selected from aliphatic, hydrogen, aromatic, or an organic functional group;
R³ is selected from aliphatic, aromatic, acyl, or sulfonyl;
R⁴ is selected from acyl, aliphatic, aromatic, or sulfonyl; and
n is an integer selected from 0 to 4;
(ii) with reference to Formula II,
R^{A}, is selected from halogen, heteroaliphatic, haloaliphatic, or an organic functional group;
R^{B} is aromatic; and
each of R^{C} and R^{D} independently is selected from hydrogen, aliphatic, or heteroaliphatic; and
m is an integer selected from 0 to 4; and
(iii) with reference to Formula III,
R′ is selected from aliphatic, aromatic, halogen, heteroaliphatic, haloaliphatic, or an organic functional group;
each R″ independently is selected from halogen, heteroaliphatic, or amino;
each R‴ independently is selected from halogen, heteroaliphatic, or amino;
p is an integer selected from 0 to 4;
q is an integer selected from 0 to 4; and
r is an integer selected from 0 or 1; and
a therapeutically acceptable excipient.

27. The composition of claim 26, formulated for oral administration or local administration to the eye.

28. (canceled)

29. (canceled)

30. (canceled)

31. (canceled)

32. The composition claim 26, wherein the composition is used to treat retinal degeneration in a subject and is formulated for local administration to an eye of the subject using intravitreal administration.

33. (canceled)

34. (canceled)

35. A method of treating retinal degeneration in a subject, comprising administering locally to the eye of the subject a therapeutically effective amount of a compound thereby treating the retinal degeneration in the subject, wherein the compound (i) maintains thickness of a nuclear layer of photoreceptors in a retina of the eye of the subject, (ii) increases expression of an opsin in the retina of the subject, (iii) increases the number of photoreceptor cells in the subject, or a combination of (i), (ii), and/or (iii); and wherein the compound is selected from a compound having a structure according to a formula selected from Formula I, II, or III 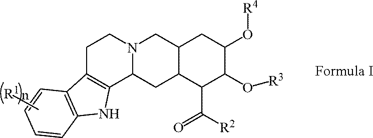 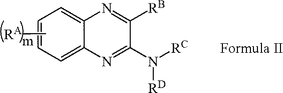 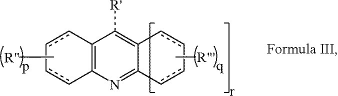
or a pharmaceutically acceptable salt, prodrug, solvate, hydrate, or tautomer thereof; or 3-(dibutylamino)-1-(1,3-dichloro-6-(trifluoromethyl)phenanthren-9-yl)propan-1-ol hydrochloride or another pharmaceutically acceptable salt, or a prodrug, solvate, hydrate, or tautomer thereof; wherein,
(i) with reference to Formula I,
R¹ is heteroaliphatic;
R² is OR⁵, or NR⁶R⁷, wherein each of R⁵, R⁶, and R⁷ independently is selected from aliphatic, hydrogen, aromatic, or an organic functional group;
R³ is selected from aliphatic, aromatic, acyl, or sulfonyl;
R⁴ is selected from acyl, aliphatic, aromatic, or sulfonyl; and
n is an integer selected from 0 to 4;
(ii) with reference to Formula II,
R^{A}, is selected from halogen, heteroaliphatic, haloaliphatic, or an organic functional group;
R^{B} is aromatic; and
each of R^{C} and R^{D} independently is selected from hydrogen, aliphatic, or heteroaliphatic; and
m is an integer selected from 0 to 4; and
(iii) with reference to Formula III,
R′ is selected from aliphatic, aromatic, halogen, heteroaliphatic, haloaliphatic, or an organic functional group;
each R″ independently is selected from halogen, heteroaliphatic, or amino;
each R‴ independently is selected from halogen, heteroaliphatic, or amino;
p is an integer selected from 0 to 4;
q is an integer selected from 0 to 4; and
r is an integer selected from 0 or 1.
